SEQUENCE LISTING (620 sequences)

SEQ ID NO: 1 - protein, 9 aa, Artificial sequence
  synthetic polypeptide
LMDMHNGQL

SEQ ID NO: 2 - protein, 9 aa, Artificial sequence
  synthetic polypeptide
RLDAMNGQL

SEQ ID NO: 3 - protein, 9 aa, Artificial sequence
  synthetic polypeptide
RMDYNNMQM

SEQ ID NO: 4 - protein, 9 aa, Artificial sequence
  synthetic polypeptide
SMDTFQGQM

SEQ ID NO: 5 - protein, 9 aa, Artificial sequence
  synthetic polypeptide
GMDYHNGHL

SEQ ID NO: 6 - protein, 9 aa, Artificial sequence
  synthetic polypeptide
YLDFHNGQL

SEQ ID NO: 7 - protein, 9 aa, Artificial sequence
  synthetic polypeptide
LMDYTNMQL

SEQ ID NO: 8 - protein, 9 aa, Artificial sequence
  synthetic polypeptide
NLDWANVQL

SEQ ID NO: 9 - protein, 9 aa, Artificial sequence
  synthetic polypeptide
MMDLHNGQL

SEQ ID NO: 10 - protein, 9 aa, Artificial sequence
  synthetic polypeptide
KMDYHEGQL

SEQ ID NO: 11 - protein, 9 aa, Artificial sequence
  synthetic polypeptide
TLDGFNGQM

SEQ ID NO: 12 - protein, 9 aa, Artificial sequence
  synthetic polypeptide
VMSHFEGQL

SEQ ID NO: 13 - protein, 9 aa, Artificial sequence
  synthetic polypeptide
AMDYLNAQL

SEQ ID NO: 14 - protein, 9 aa, Artificial sequence
  synthetic polypeptide
QLDWNNMQM

SEQ ID NO: 15 - protein, 9 aa, Artificial sequence
  synthetic polypeptide
RMGYHNGQL

SEQ ID NO: 16 - protein, 9 aa, Artificial sequence
  synthetic polypeptide
RMDRFNGQL

SEQ ID NO: 17 - protein, 9 aa, Artificial sequence
  synthetic polypeptide
AMSYDNMQL

SEQ ID NO: 18 - protein, 9 aa, Artificial sequence
  synthetic polypeptide
VMTHNNMQL

SEQ ID NO: 19 - protein, 9 aa, Artificial sequence
  synthetic polypeptide
NMSWQNMQL

SEQ ID NO: 20 - protein, 9 aa, Artificial sequence
  synthetic polypeptide
RMDVNNMQL

SEQ ID NO: 21 - protein, 9 aa, Artificial sequence
  synthetic polypeptide
NLDWNNVQM

SEQ ID NO: 22 - protein, 9 aa, Artificial sequence
  synthetic polypeptide
ELDWFNSQL

SEQ ID NO: 23 - protein, 9 aa, Artificial sequence
  synthetic polypeptide
CMDVFNGQL

SEQ ID NO: 24 - protein, 9 aa, Artificial sequence
  synthetic polypeptide
GMSYSNMQL

SEQ ID NO: 25 - protein, 9 aa, Artificial sequence
  synthetic polypeptide
SMTWMNGQL

SEQ ID NO: 26 - protein, 9 aa, Artificial sequence
  synthetic polypeptide
SMDRFNGQM

SEQ ID NO: 27 - protein, 9 aa, Artificial sequence
  synthetic polypeptide
VLDQHNGQL

SEQ ID NO: 28 - protein, 9 aa, Artificial sequence
  synthetic polypeptide
HMDFNNVQM

SEQ ID NO: 29 - protein, 9 aa, Artificial sequence
  synthetic polypeptide
SMSWMNGQL

SEQ ID NO: 30 - protein, 9 aa, Artificial sequence
  synthetic polypeptide
MLDWNNVQL

SEQ ID NO: 31 - protein, 9 aa, Artificial sequence
  synthetic polypeptide
EMDVHNGQM

SEQ ID NO: 32 - protein, 9 aa, Artificial sequence
  synthetic polypeptide
KMHWFNGQL

SEQ ID NO: 33 - protein, 9 aa, Artificial sequence
  synthetic polypeptide
SMDSLNGQL

SEQ ID NO: 34 - protein, 9 aa, Artificial sequence
  synthetic polypeptide
VMTYQNGQL

SEQ ID NO: 35 - protein, 9 aa, Artificial sequence
  synthetic polypeptide
VMDHLNGQL

SEQ ID NO: 36 - protein, 9 aa, Artificial sequence
  synthetic polypeptide
WMSDFQGQL

SEQ ID NO: 37 - protein, 9 aa, Artificial sequence
  synthetic polypeptide
RLDSFNGQL

SEQ ID NO: 38 - protein, 9 aa, Artificial sequence
  synthetic polypeptide
SMDSWNGQM

SEQ ID NO: 39 - protein, 9 aa, Artificial sequence
  synthetic polypeptide
TMDWHSGQL

SEQ ID NO: 40 - protein, 9 aa, Artificial sequence
  synthetic polypeptide
KLDIWNGQL

SEQ ID NO: 41 - protein, 9 aa, Artificial sequence
  synthetic polypeptide
TMDFYQGQL

SEQ ID NO: 42 - protein, 9 aa, Artificial sequence
  synthetic polypeptide
KMDYFSGQL

SEQ ID NO: 43 - protein, 9 aa, Artificial sequence
  synthetic polypeptide
YLDYRNMQL

SEQ ID NO: 44 - protein, 9 aa, Artificial sequence
  synthetic polypeptide
EMDHLNMQL

SEQ ID NO: 45 - protein, 9 aa, Artificial sequence
  synthetic polypeptide
HMDINNMQM

SEQ ID NO: 46 - protein, 9 aa, Artificial sequence
  synthetic polypeptide
SLDWFNSQL

SEQ ID NO: 47 - protein, 9 aa, Artificial sequence
  synthetic polypeptide
RMDWLQAQL

SEQ ID NO: 48 - protein, 9 aa, Artificial sequence
  synthetic polypeptide
FLDFRNGQM

SEQ ID NO: 49 - protein, 9 aa, Artificial sequence
  synthetic polypeptide
EMMWWNGQV

SEQ ID NO: 50 - protein, 9 aa, Artificial sequence
  synthetic polypeptide
TMEWFNGHL

SEQ ID NO: 51 - protein, 9 aa, Artificial sequence
  synthetic polypeptide
TMDTLNAQL

SEQ ID NO: 52 - protein, 9 aa, Artificial sequence
  synthetic polypeptide
FMDSFNGQM

SEQ ID NO: 53 - protein, 9 aa, Artificial sequence
  synthetic polypeptide
NMMWFQGQL

SEQ ID NO: 54 - protein, 9 aa, Artificial sequence
  synthetic polypeptide
NMGFENMQL

SEQ ID NO: 55 - protein, 9 aa, Artificial sequence
  synthetic polypeptide
NMDYINVQL

SEQ ID NO: 56 - protein, 9 aa, Artificial sequence
  synthetic polypeptide
EMDWSNLQL

SEQ ID NO: 57 - protein, 9 aa, Artificial sequence
  synthetic polypeptide
LMGIHNGQL

SEQ ID NO: 58 - protein, 9 aa, Artificial sequence
  synthetic polypeptide
EMSWFSGQL

SEQ ID NO: 59 - protein, 9 aa, Artificial sequence
  synthetic polypeptide
VMDLFQGQM

SEQ ID NO: 60 - protein, 9 aa, Artificial sequence
  synthetic polypeptide
LLDVHNMQL

SEQ ID NO: 61 - protein, 9 aa, Artificial sequence
  synthetic polypeptide
KMDYNNVQM

SEQ ID NO: 62 - protein, 9 aa, Artificial sequence
  synthetic polypeptide
SMDYNNVQM

SEQ ID NO: 63 - protein, 9 aa, Artificial sequence
  synthetic polypeptide
LMENFQGQL

SEQ ID NO: 64 - protein, 9 aa, Artificial sequence
  synthetic polypeptide
RMSFHNGQL

SEQ ID NO: 65 - protein, 9 aa, Artificial sequence
  synthetic polypeptide
SMMYMNGQL

SEQ ID NO: 66 - protein, 9 aa, Artificial sequence
  synthetic polypeptide
RMEWQNAQL

SEQ ID NO: 67 - protein, 9 aa, Artificial sequence
  synthetic polypeptide
VMSHQNMQL

SEQ ID NO: 68 - protein, 9 aa, Artificial sequence
  synthetic polypeptide
MMDFFDGQM

SEQ ID NO: 69 - protein, 9 aa, Artificial sequence
  synthetic polypeptide
IMSHQNMQL

SEQ ID NO: 70 - protein, 9 aa, Artificial sequence
  synthetic polypeptide
HMEFMNMQL

SEQ ID NO: 71 - protein, 9 aa, Artificial sequence
  synthetic polypeptide
NMDTYNGQM

SEQ ID NO: 72 - protein, 9 aa, Artificial sequence
  synthetic polypeptide
NLDYTNGQL

SEQ ID NO: 73 - protein, 9 aa, Artificial sequence
  synthetic polypeptide
SMTWENMQL

SEQ ID NO: 74 - protein, 9 aa, Artificial sequence
  synthetic polypeptide
AMTFHNGQL

SEQ ID NO: 75 - protein, 9 aa, Artificial sequence
  synthetic polypeptide
SMDFTNAQM

SEQ ID NO: 76 - protein, 9 aa, Artificial sequence
  synthetic polypeptide
NMSTRDERM

SEQ ID NO: 77 - protein, 9 aa, Artificial sequence
  synthetic polypeptide
SMTFENMQL

SEQ ID NO: 78 - protein, 9 aa, Artificial sequence
  synthetic polypeptide
EMDWWNGHL

SEQ ID NO: 79 - protein, 9 aa, Artificial sequence
  synthetic polypeptide
TMDDNNGQL

SEQ ID NO: 80 - protein, 9 aa, Artificial sequence
  synthetic polypeptide
LMDENNMQL

SEQ ID NO: 81 - protein, 9 aa, Artificial sequence
  synthetic polypeptide
EMTNWNGQL

SEQ ID NO: 82 - protein, 9 aa, Artificial sequence
  synthetic polypeptide
YMDYHNGHM

SEQ ID NO: 83 - protein, 9 aa, Artificial sequence
  synthetic polypeptide
KMTWNNMQM

SEQ ID NO: 84 - protein, 9 aa, Artificial sequence
  synthetic polypeptide
YMTHLNGQL

SEQ ID NO: 85 - protein, 9 aa, Artificial sequence
  synthetic polypeptide
EMTWTNAQM

SEQ ID NO: 86 - protein, 9 aa, Artificial sequence
  synthetic polypeptide
KMNNFEGQL

SEQ ID NO: 87 - protein, 9 aa, Artificial sequence
  synthetic polypeptide
MMDLYNGQL

SEQ ID NO: 88 - protein, 9 aa, Artificial sequence
  synthetic polypeptide
VLDNNNMQL

SEQ ID NO: 89 - protein, 9 aa, Artificial sequence
  synthetic polypeptide
KLAWFNGQL

SEQ ID NO: 90 - protein, 9 aa, Artificial sequence
  synthetic polypeptide
NLDHNNGQM

SEQ ID NO: 91 - protein, 9 aa, Artificial sequence
  synthetic polypeptide
LMDNSNMQL

SEQ ID NO: 92 - protein, 9 aa, Artificial sequence
  synthetic polypeptide
NMDYNNVQL

SEQ ID NO: 93 - protein, 9 aa, Artificial sequence
  synthetic polypeptide
RMDYNNVQM

SEQ ID NO: 94 - protein, 9 aa, Artificial sequence
  synthetic polypeptide
EMEIMNMQL

SEQ ID NO: 95 - protein, 9 aa, Artificial sequence
  synthetic polypeptide
YMDRFQGQL

SEQ ID NO: 96 - protein, 9 aa, Artificial sequence
  synthetic polypeptide
YMNVFEGQL

SEQ ID NO: 97 - protein, 9 aa, Artificial sequence
  synthetic polypeptide
LMDTFNAQM

SEQ ID NO: 98 - protein, 9 aa, Artificial sequence
  synthetic polypeptide
GMDYHNGQL

SEQ ID NO: 99 - protein, 9 aa, Artificial sequence
  synthetic polypeptide
MLDLYNGQL

SEQ ID NO: 100 - protein, 9 aa, Artificial sequence
  synthetic polypeptide
RLSWFQGQL

SEQ ID NO: 101 - protein, 9 aa, Artificial sequence
  synthetic polypeptide
VLNGFDGQL

SEQ ID NO: 102 - protein, 9 aa, Artificial sequence
  synthetic polypeptide
SMGWEQLQL

SEQ ID NO: 103 - protein, 9 aa, Artificial sequence
  synthetic polypeptide
SMTWFTGQL

SEQ ID NO: 104 - protein, 9 aa, Artificial sequence
  synthetic polypeptide
WMDISNMQL

SEQ ID NO: 105 - protein, 9 aa, Artificial sequence
  synthetic polypeptide
TMQWQNAQL

SEQ ID NO: 106 - protein, 9 aa, Artificial sequence
  synthetic polypeptide
SMTVFNGQL

SEQ ID NO: 107 - protein, 9 aa, Artificial sequence
  synthetic polypeptide
NMDMHNMQL

SEQ ID NO: 108 - protein, 9 aa, Artificial sequence
  synthetic polypeptide
RMSSFDGQL

SEQ ID NO: 109 - protein, 9 aa, Artificial sequence
  synthetic polypeptide
YMSFDNVQL

SEQ ID NO: 110 - protein, 9 aa, Artificial sequence
  synthetic polypeptide
LMSGFDGQL

SEQ ID NO: 111 - protein, 9 aa, Artificial sequence
  synthetic polypeptide
YLDYLNMQL

SEQ ID NO: 112 - protein, 9 aa, Artificial sequence
  synthetic polypeptide
SMDYNNIQM

SEQ ID NO: 113 - protein, 9 aa, Artificial sequence
  synthetic polypeptide
GMDTHNGQL

SEQ ID NO: 114 - protein, 9 aa, Artificial sequence
  synthetic polypeptide
LMDMHNGHL

SEQ ID NO: 115 - protein, 9 aa, Artificial sequence
  synthetic polypeptide
SLNYWEGQL

SEQ ID NO: 116 - protein, 9 aa, Artificial sequence
  synthetic polypeptide
ALNHFEGQL

SEQ ID NO: 117 - protein, 9 aa, Artificial sequence
  synthetic polypeptide
AMDNMNGQL

SEQ ID NO: 118 - protein, 9 aa, Artificial sequence
  synthetic polypeptide
RMGIFNGQL

SEQ ID NO: 119 - protein, 9 aa, Artificial sequence
  synthetic polypeptide
NLDWSNAQL

SEQ ID NO: 120 - protein, 9 aa, Artificial sequence
  synthetic polypeptide
RMDHMNGHL

SEQ ID NO: 121 - protein, 9 aa, Artificial sequence
  synthetic polypeptide
MMSPFNGQL

SEQ ID NO: 122 - protein, 9 aa, Artificial sequence
  synthetic polypeptide
TMNSWNGQL

SEQ ID NO: 123 - protein, 9 aa, Artificial sequence
  synthetic polypeptide
SMNWQNGQL

SEQ ID NO: 124 - protein, 9 aa, Artificial sequence
  synthetic polypeptide
IMETFNGQM

SEQ ID NO: 125 - protein, 9 aa, Artificial sequence
  synthetic polypeptide
YLDNNNMQM

SEQ ID NO: 126 - protein, 9 aa, Artificial sequence
  synthetic polypeptide
QMDLMKTYL

SEQ ID NO: 127 - protein, 9 aa, Artificial sequence
  synthetic polypeptide
GLDWINGQL

SEQ ID NO: 128 - protein, 9 aa, Artificial sequence
  synthetic polypeptide
RLTYLNGQL

SEQ ID NO: 129 - protein, 9 aa, Artificial sequence
  synthetic polypeptide
AMDDWNGQM

SEQ ID NO: 130 - protein, 9 aa, Artificial sequence
  synthetic polypeptide
NLDWQNMQM

SEQ ID NO: 131 - protein, 9 aa, Artificial sequence
  synthetic polypeptide
TMDYNNAQM

SEQ ID NO: 132 - protein, 9 aa, Artificial sequence
  synthetic polypeptide
TMDENNMQL

SEQ ID NO: 133 - protein, 9 aa, Artificial sequence
  synthetic polypeptide
WMDDINGQL

SEQ ID NO: 134 - protein, 9 aa, Artificial sequence
  synthetic polypeptide
MLDYMNAQM

SEQ ID NO: 135 - protein, 9 aa, Artificial sequence
  synthetic polypeptide
AMDKHNGQM

SEQ ID NO: 136 - protein, 9 aa, Artificial sequence
  synthetic polypeptide
KMDWRVVQM

SEQ ID NO: 137 - protein, 9 aa, Artificial sequence
  synthetic polypeptide
RMDYTNMQL

SEQ ID NO: 138 - protein, 9 aa, Artificial sequence
  synthetic polypeptide
RMDHSNMQM

SEQ ID NO: 139 - protein, 9 aa, Artificial sequence
  synthetic polypeptide
TLEIHNGQL

SEQ ID NO: 140 - protein, 9 aa, Artificial sequence
  synthetic polypeptide
LMDMHNMQM

SEQ ID NO: 141 - protein, 9 aa, Artificial sequence
  synthetic polypeptide
SLTYFNGQM

SEQ ID NO: 142 - protein, 9 aa, Artificial sequence
  synthetic polypeptide
YMDMHNGQL

SEQ ID NO: 143 - protein, 9 aa, Artificial sequence
  synthetic polypeptide
NMDRHNGQM

SEQ ID NO: 144 - protein, 9 aa, Artificial sequence
  synthetic polypeptide
NMDRNNMQL

SEQ ID NO: 145 - protein, 9 aa, Artificial sequence
  synthetic polypeptide
TLDVHNMQL

SEQ ID NO: 146 - protein, 9 aa, Artificial sequence
  synthetic polypeptide
RLSTFEGQL

SEQ ID NO: 147 - protein, 9 aa, Artificial sequence
  synthetic polypeptide
QMDTMNGQL

SEQ ID NO: 148 - protein, 9 aa, Artificial sequence
  synthetic polypeptide
KMDYHNGHL

SEQ ID NO: 149 - protein, 9 aa, Artificial sequence
  synthetic polypeptide
IMDWSNVQM

SEQ ID NO: 150 - protein, 9 aa, Artificial sequence
  synthetic polypeptide
KLDAFNGQM

SEQ ID NO: 151 - protein, 9 aa, Artificial sequence
  synthetic polypeptide
CLSESLQWV

SEQ ID NO: 152 - protein, 9 aa, Artificial sequence
  synthetic polypeptide
SMCYQNMQL

SEQ ID NO: 153 - protein, 9 aa, Artificial sequence
  synthetic polypeptide
LMTCAGNDM

SEQ ID NO: 154 - protein, 9 aa, Artificial sequence
  synthetic polypeptide
KLDVFNAQL

SEQ ID NO: 155 - protein, 9 aa, Artificial sequence
  synthetic polypeptide
LMDYNNMQM

SEQ ID NO: 156 - protein, 9 aa, Artificial sequence
  synthetic polypeptide
YLDFHNGHL

SEQ ID NO: 157 - protein, 9 aa, Artificial sequence
  synthetic polypeptide
AMDMHNGQL

SEQ ID NO: 158 - protein, 9 aa, Artificial sequence
  synthetic polypeptide
SMNYYDGQL

SEQ ID NO: 159 - protein, 9 aa, Artificial sequence
  synthetic polypeptide
YMDWSNSQM

SEQ ID NO: 160 - protein, 9 aa, Artificial sequence
  synthetic polypeptide
TLDHMNAQM

SEQ ID NO: 161 - protein, 9 aa, Artificial sequence
  synthetic polypeptide
HMNYFDGQM

SEQ ID NO: 162 - protein, 9 aa, Artificial sequence
  synthetic polypeptide
TLCYNNMQL

SEQ ID NO: 163 - protein, 9 aa, Artificial sequence
  synthetic polypeptide
FMDDFSGQL

SEQ ID NO: 164 - protein, 9 aa, Artificial sequence
  synthetic polypeptide
QLDWNNVQL

SEQ ID NO: 165 - protein, 9 aa, Artificial sequence
  synthetic polypeptide
TLDFRNMQL

SEQ ID NO: 166 - protein, 9 aa, Artificial sequence
  synthetic polypeptide
VLLRDASWM

SEQ ID NO: 167 - protein, 9 aa, Artificial sequence
  synthetic polypeptide
TMEWFNGQM

SEQ ID NO: 168 - protein, 9 aa, Artificial sequence
  synthetic polypeptide
FMDFNSGQL

SEQ ID NO: 169 - protein, 9 aa, Artificial sequence
  synthetic polypeptide
SMDMHNGQL

SEQ ID NO: 170 - protein, 9 aa, Artificial sequence
  synthetic polypeptide
RLQDISGVM

SEQ ID NO: 171 - protein, 9 aa, Artificial sequence
  synthetic polypeptide
ELMAWNGQL

SEQ ID NO: 172 - protein, 9 aa, Artificial sequence
  synthetic polypeptide
NLDWNNMQM

SEQ ID NO: 173 - protein, 9 aa, Artificial sequence
  synthetic polypeptide
RMDYLNAQL

SEQ ID NO: 174 - protein, 9 aa, Artificial sequence
  synthetic polypeptide
FMDFHNGQL

SEQ ID NO: 175 - protein, 9 aa, Artificial sequence
  synthetic polypeptide
MMDLHNGHL

SEQ ID NO: 176 - protein, 9 aa, Artificial sequence
  synthetic polypeptide
LMDTFQGQM

SEQ ID NO: 177 - protein, 9 aa, Artificial sequence
  synthetic polypeptide
AMDFHNGQL

SEQ ID NO: 178 - protein, 9 aa, Artificial sequence
  synthetic polypeptide
TMDFSNIQL

SEQ ID NO: 179 - protein, 9 aa, Artificial sequence
  synthetic polypeptide
GMDDHNMQL

SEQ ID NO: 180 - protein, 9 aa, Artificial sequence
  synthetic polypeptide
KMHYFNGQM

SEQ ID NO: 181 - protein, 9 aa, Artificial sequence
  synthetic polypeptide
YMDYHNGQL

SEQ ID NO: 182 - protein, 9 aa, Artificial sequence
  synthetic polypeptide
RMDYNNGHL

SEQ ID NO: 183 - protein, 9 aa, Artificial sequence
  synthetic polypeptide
LMDYHEGQL

SEQ ID NO: 184 - protein, 9 aa, Artificial sequence
  synthetic polypeptide
RMDRFNGQM

SEQ ID NO: 185 - protein, 9 aa, Artificial sequence
  synthetic polypeptide
RMDVNNGQL

SEQ ID NO: 186 - protein, 9 aa, Artificial sequence
  synthetic polypeptide
GMDTANMQL

SEQ ID NO: 187 - protein, 9 aa, Artificial sequence
  synthetic polypeptide
MLDYMNGQL

SEQ ID NO: 188 - protein, 9 aa, Artificial sequence
  synthetic polypeptide
KMTFHNAQL

SEQ ID NO: 189 - protein, 9 aa, Artificial sequence
  synthetic polypeptide
FMDFNNVQM

SEQ ID NO: 190 - protein, 9 aa, Artificial sequence
  synthetic polypeptide
SLDHFQGHL

SEQ ID NO: 191 - protein, 9 aa, Artificial sequence
  synthetic polypeptide
TMDFYQGQL

SEQ ID NO: 192 - protein, 9 aa, Artificial sequence
  synthetic polypeptide
KMDYFSGQL

SEQ ID NO: 193 - protein, 9 aa, Artificial sequence
  synthetic polypeptide
SMDWFQGQM

SEQ ID NO: 194 - protein, 9 aa, Artificial sequence
  synthetic polypeptide
LMDYWQGQL

SEQ ID NO: 195 - protein, 9 aa, Artificial sequence
  synthetic polypeptide
NMMWFQGQL

SEQ ID NO: 196 - protein, 9 aa, Artificial sequence
  synthetic polypeptide
KMHWFNGQL

SEQ ID NO: 197 - protein, 9 aa, Artificial sequence
  synthetic polypeptide
TMDYWQGHL

SEQ ID NO: 198 - protein, 9 aa, Artificial sequence
  synthetic polypeptide
RMDRFNGQL

SEQ ID NO: 199 - protein, 9 aa, Artificial sequence
  synthetic polypeptide
SMDTFQGQM

SEQ ID NO: 200 - protein, 9 aa, Artificial sequence
  synthetic polypeptide
VMSHFEGQL

SEQ ID NO: 201 - protein, 9 aa, Artificial sequence
  synthetic polypeptide
LMDYTNMQL

SEQ ID NO: 202 - protein, 9 aa, Artificial sequence
  synthetic polypeptide
KMDYHIGQM

SEQ ID NO: 203 - protein, 9 aa, Artificial sequence
  synthetic polypeptide
VMDHFQAQL

SEQ ID NO: 204 - protein, 9 aa, Artificial sequence
  synthetic polypeptide
NMGFENMQL

SEQ ID NO: 205 - protein, 9 aa, Artificial sequence
  synthetic polypeptide
YLDHKTLRL

SEQ ID NO: 206 - protein, 9 aa, Artificial sequence
  synthetic polypeptide
TMDYWQGQL

SEQ ID NO: 207 - protein, 9 aa, Artificial sequence
  synthetic polypeptide
KMRMNRHKL

SEQ ID NO: 208 - protein, 9 aa, Artificial sequence
  synthetic polypeptide
YMDRFQGQM

SEQ ID NO: 209 - protein, 9 aa, Artificial sequence
  synthetic polypeptide
SMDFFNSQL

SEQ ID NO: 210 - protein, 9 aa, Artificial sequence
  synthetic polypeptide
NMEEYCALV

SEQ ID NO: 211 - protein, 9 aa, Artificial sequence
  synthetic polypeptide
SMDFYQGQL

SEQ ID NO: 212 - protein, 9 aa, Artificial sequence
  synthetic polypeptide
SMDWFQGQL

SEQ ID NO: 213 - protein, 9 aa, Artificial sequence
  synthetic polypeptide
NMMWFQGQM

SEQ ID NO: 214 - protein, 9 aa, Artificial sequence
  synthetic polypeptide
AMYKLSGLM

SEQ ID NO: 215 - protein, 9 aa, Artificial sequence
  synthetic polypeptide
HMEYRYANM

SEQ ID NO: 216 - protein, 9 aa, Artificial sequence
  synthetic polypeptide
LMDYFSGQL

SEQ ID NO: 217 - protein, 9 aa, Artificial sequence
  synthetic polypeptide
TMDWFQGQM

SEQ ID NO: 218 - protein, 9 aa, Artificial sequence
  synthetic polypeptide
FMSVAKFVV

SEQ ID NO: 219 - protein, 9 aa, Artificial sequence
  synthetic polypeptide
RLDYHNMQL

SEQ ID NO: 220 - protein, 9 aa, Artificial sequence
  synthetic polypeptide
LMDFYQGQL

SEQ ID NO: 221 - protein, 9 aa, Artificial sequence
  synthetic polypeptide
LMDYWQGHL

SEQ ID NO: 222 - protein, 9 aa, Artificial sequence
  synthetic polypeptide
TMDFYQGQM

SEQ ID NO: 223 - protein, 9 aa, Artificial sequence
  synthetic polypeptide
KMLSIDVVM

SEQ ID NO: 224 - protein, 9 aa, Artificial sequence
  synthetic polypeptide
SMDYFSGQL

SEQ ID NO: 225 - protein, 9 aa, Artificial sequence
  synthetic polypeptide
KMKNHHTKV

SEQ ID NO: 226 - protein, 9 aa, Artificial sequence
  synthetic polypeptide
SMDYWQGQL

SEQ ID NO: 227 - protein, 9 aa, Artificial sequence
  synthetic polypeptide
KLHRHKQHM

SEQ ID NO: 228 - protein, 9 aa, Artificial sequence
  synthetic polypeptide
LMDWFQGQM

SEQ ID NO: 229 - protein, 9 aa, Artificial sequence
  synthetic polypeptide
KMTSWWDML

SEQ ID NO: 230 - protein, 9 aa, Artificial sequence
  synthetic polypeptide
DMDWFQGQM

SEQ ID NO: 231 - protein, 9 aa, Artificial sequence
  synthetic polypeptide
MLYELTEHL

SEQ ID NO: 232 - protein, 9 aa, Artificial sequence
  synthetic polypeptide
SMDWFNGQL

SEQ ID NO: 233 - protein, 9 aa, Artificial sequence
  synthetic polypeptide
RLHRRDNLM

SEQ ID NO: 234 - protein, 9 aa, Artificial sequence
  synthetic polypeptide
DMDYWQGQL

SEQ ID NO: 235 - protein, 9 aa, Artificial sequence
  synthetic polypeptide
KMDYTNMQL

SEQ ID NO: 236 - protein, 9 aa, Artificial sequence
  synthetic polypeptide
TMDYWQGQM

SEQ ID NO: 237 - protein, 9 aa, Artificial sequence
  synthetic polypeptide
FMGVSYEMM

SEQ ID NO: 238 - protein, 9 aa, Artificial sequence
  synthetic polypeptide
LMDYWQGQM

SEQ ID NO: 239 - protein, 9 aa, Artificial sequence
  synthetic polypeptide
SMDTFQGQL

SEQ ID NO: 240 - protein, 9 aa, Artificial sequence
  synthetic polypeptide
KMHGHKHYM

SEQ ID NO: 241 - protein, 9 aa, Artificial sequence
  synthetic polypeptide
KMHWFQGQM

SEQ ID NO: 242 - protein, 9 aa, Artificial sequence
  synthetic polypeptide
SLDYFNSQL

SEQ ID NO: 243 - protein, 9 aa, Artificial sequence
  synthetic polypeptide
YMDRFQGQL

SEQ ID NO: 244 - protein, 9 aa, Artificial sequence
  synthetic polypeptide
RMWSDRMDL

SEQ ID NO: 245 - protein, 9 aa, Artificial sequence
  synthetic polypeptide
KMDYFNSQL

SEQ ID NO: 246 - protein, 9 aa, Artificial sequence
  synthetic polypeptide
YMHSHSVLL

SEQ ID NO: 247 - protein, 9 aa, Artificial sequence
  synthetic polypeptide
DMDYFSGQL

SEQ ID NO: 248 - protein, 9 aa, Artificial sequence
  synthetic polypeptide
SMDWFQGHL

SEQ ID NO: 249 - protein, 9 aa, Artificial sequence
  synthetic polypeptide
VMDLFQGQM

SEQ ID NO: 250 - protein, 9 aa, Artificial sequence
  synthetic polypeptide
NMESWLSMM

SEQ ID NO: 251 - protein, 9 aa, Artificial sequence
  synthetic polypeptide
RMDRFQGQM

SEQ ID NO: 252 - protein, 9 aa, Artificial sequence
  synthetic polypeptide
SMEISNLNM

SEQ ID NO: 253 - protein, 9 aa, Artificial sequence
  synthetic polypeptide
DMERALMNL

SEQ ID NO: 254 - protein, 9 aa, Artificial sequence
  synthetic polypeptide
DMDTFQGQM

SEQ ID NO: 255 - protein, 9 aa, Artificial sequence
  synthetic polypeptide
KMKKNHDHM

SEQ ID NO: 256 - protein, 9 aa, Artificial sequence
  synthetic polypeptide
KMREMPVKM

SEQ ID NO: 257 - protein, 9 aa, Artificial sequence
  synthetic polypeptide
MMDFFNAQM

SEQ ID NO: 258 - protein, 210 aa, Artificial sequence
  synthetic polypeptide
QKVTQAQTEISVVEKEDVTLDCVYETRDTTYYLFWYKQPPSGELVFLIRRNSFDEQNEISGRYSWNFQKSTSSFNFTITASQVVDSAVYFCALSEARGGATNKLIFGTGTLLAVQPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 259 - protein, 241 aa, Artificial sequence
  synthetic polypeptide
EITQSPRHKITETGRQVTLACHQTWNHNNMFWYRQDLGHGLRLIHYSYGVQDTNKGEVSDGYSVSRSNTEDLPLTLESAASSQTSVYFCASSRDTVNTEAFFGQGTRLTVVEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRAD

SEQ ID NO: 260 - protein, 241 aa, Artificial sequence
  synthetic polypeptide
EITQSPRHKITETGRQVTLACHQTWNHNNMFWYRQDLGHGLRLIHYSYGVQDTNKGEVSDGYSVSRSNTEDLPLTLESAASSQTSVYFCASSRDFVSNEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRAD

SEQ ID NO: 261 - protein, 16 aa, Artificial sequence
  synthetic polypeptide
CALSEARGGATNKLIF

SEQ ID NO: 262 - protein, 14 aa, Artificial sequence
  synthetic polypeptide
CASSRDTVNTEAFF

SEQ ID NO: 263 - protein, 14 aa, Artificial sequence
  synthetic polypeptide
CASSRDFVSNEQYF

SEQ ID NO: 264 - protein, 10 aa, Artificial sequence
  synthetic polypeptide
ELAGIGILTV

SEQ ID NO: 265 - protein, 10 aa, Artificial sequence
  synthetic polypeptide
ALDPHSGHFV

SEQ ID NO: 266 - protein, 9 aa, Artificial sequence
  synthetic polypeptide
MMDFFNAQM

SEQ ID NO: 267 - protein, 10 aa, Artificial sequence
  synthetic polypeptide
EAAGIGILTV

SEQ ID NO: 268 - protein, 10 aa, Artificial sequence
  synthetic polypeptide
ALLETPSLLL

SEQ ID NO: 269 - protein, 10 aa, Artificial sequence
  synthetic polypeptide
ALDSRSEHFM

SEQ ID NO: 270 - protein, 8 aa, Artificial sequence
  synthetic polypeptide
EYGVSYEW

SEQ ID NO: 271 - protein, 8 aa, Artificial sequence
  synthetic polypeptide
EMGVSYEM

SEQ ID NO: 272 - protein, 9 aa, Artificial sequence
  synthetic polypeptide
LLEDLDWDV

SEQ ID NO: 273 - protein, 9 aa, Artificial sequence
  synthetic polypeptide
NMEYMTWDV

SEQ ID NO: 274 - protein, 9 aa, Artificial sequence
  synthetic polypeptide
TMETIDWKV

SEQ ID NO: 275 - protein, 9 aa, Artificial sequence
  synthetic polypeptide
VLEEVDWLI

SEQ ID NO: 276 - protein, 9 aa, Artificial sequence
  synthetic polypeptide
KLEQLDWTV

SEQ ID NO: 277 - protein, 9 aa, Artificial sequence
  synthetic polypeptide
TLEELDWCL

SEQ ID NO: 278 - protein, 9 aa, Artificial sequence
  synthetic polypeptide
NVEYYDIKL

SEQ ID NO: 279 - protein, 9 aa, Artificial sequence
  synthetic polypeptide
MMDFFNAQM

SEQ ID NO: 280 - protein, 8 aa, Artificial sequence
  synthetic polypeptide
VLDFQGQL

SEQ ID NO: 281 - protein, 9 aa, Artificial sequence
  synthetic polypeptide
MMDFFNAQM

SEQ ID NO: 282 - protein, 9 aa, Artificial sequence
  synthetic polypeptide
MMDFFNAQM

SEQ ID NO: 283 - protein, 10 aa, Artificial sequence
  synthetic polypeptide
SMLGIGIVPV

SEQ ID NO: 284 - protein, 10 aa, Artificial sequence
  synthetic polypeptide
SMAGIGIVDV

SEQ ID NO: 285 - protein, 10 aa, Artificial sequence
  synthetic polypeptide
NMGGLGIMPV

SEQ ID NO: 286 - protein, 10 aa, Artificial sequence
  synthetic polypeptide
NLSNLGILPV

SEQ ID NO: 287 - protein, 10 aa, Artificial sequence
  synthetic polypeptide
SMLGIGIYPV

SEQ ID NO: 288 - protein, 10 aa, Artificial sequence
  synthetic polypeptide
TMAGIGVHVV

SEQ ID NO: 289 - protein, 10 aa, Artificial sequence
  synthetic polypeptide
SMAGIGTLVV

SEQ ID NO: 290 - protein, 10 aa, Artificial sequence
  synthetic polypeptide
SMSGLGILPM

SEQ ID NO: 291 - protein, 10 aa, Artificial sequence
  synthetic polypeptide
SMAGIGIVPV

SEQ ID NO: 292 - protein, 10 aa, Artificial sequence
  synthetic polypeptide
SMLGIGIVDV

SEQ ID NO: 293 - protein, 10 aa, Artificial sequence
  synthetic polypeptide
NMAGIGMGTV

SEQ ID NO: 294 - protein, 10 aa, Artificial sequence
  synthetic polypeptide
SMLGIGILPV

SEQ ID NO: 295 - protein, 10 aa, Artificial sequence
  synthetic polypeptide
SLSGIGISAV

SEQ ID NO: 296 - protein, 10 aa, Artificial sequence
  synthetic polypeptide
DLAGLGLYPV

SEQ ID NO: 297 - protein, 10 aa, Artificial sequence
  synthetic polypeptide
NMAGIGIIQV

SEQ ID NO: 298 - protein, 10 aa, Artificial sequence
  synthetic polypeptide
NMGGLGILPV

SEQ ID NO: 299 - protein, 10 aa, Artificial sequence
  synthetic polypeptide
SMAGIGIYPV

SEQ ID NO: 300 - protein, 10 aa, Artificial sequence
  synthetic polypeptide
NLSNLGIVPV

SEQ ID NO: 301 - protein, 10 aa, Artificial sequence
  synthetic polypeptide
IMLGIGIDTL

SEQ ID NO: 302 - protein, 10 aa, Artificial sequence
  synthetic polypeptide
NLSNLGIMPV

SEQ ID NO: 303 - protein, 10 aa, Artificial sequence
  synthetic polypeptide
SMLGIGIVLV

SEQ ID NO: 304 - protein, 10 aa, Artificial sequence
  synthetic polypeptide
SMAGIGVHVV

SEQ ID NO: 305 - protein, 10 aa, Artificial sequence
  synthetic polypeptide
NMAGIGILTV

SEQ ID NO: 306 - protein, 10 aa, Artificial sequence
  synthetic polypeptide
MMAGIGIVDV

SEQ ID NO: 307 - protein, 10 aa, Artificial sequence
  synthetic polypeptide
NMGGLGIVPV

SEQ ID NO: 308 - protein, 10 aa, Artificial sequence
  synthetic polypeptide
SMLGIKIVPV

SEQ ID NO: 309 - protein, 10 aa, Artificial sequence
  synthetic polypeptide
ELSGLGIQTV

SEQ ID NO: 310 - protein, 10 aa, Artificial sequence
  synthetic polypeptide
SMLGIGILPM

SEQ ID NO: 311 - protein, 10 aa, Artificial sequence
  synthetic polypeptide
SMAGIGILPV

SEQ ID NO: 312 - protein, 10 aa, Artificial sequence
  synthetic polypeptide
SMLGIGIVPV

SEQ ID NO: 313 - protein, 10 aa, Artificial sequence
  synthetic polypeptide
EAAGIGILTV

SEQ ID NO: 314 - protein, 10 aa, Artificial sequence
  synthetic polypeptide
TLGGIGLVTV

SEQ ID NO: 315 - protein, 10 aa, Artificial sequence
  synthetic polypeptide
ILLGIGIYAL

SEQ ID NO: 316 - protein, 10 aa, Artificial sequence
  synthetic polypeptide
ILSGIGVSQV

SEQ ID NO: 317 - protein, 10 aa, Artificial sequence
  synthetic polypeptide
IMGNLGLIAV

SEQ ID NO: 318 - protein, 10 aa, Artificial sequence
  synthetic polypeptide
MAGNLGIITL

SEQ ID NO: 319 - protein, 10 aa, Artificial sequence
  synthetic polypeptide
IMGNLGLIVL

SEQ ID NO: 320 - protein, 10 aa, Artificial sequence
  synthetic polypeptide
ILAGLGTSLL

SEQ ID NO: 321 - protein, 10 aa, Artificial sequence
  synthetic polypeptide
ELGGLKISTL

SEQ ID NO: 322 - protein, 10 aa, Artificial sequence
  synthetic polypeptide
MMWDRGMGLL

SEQ ID NO: 323 - protein, 10 aa, Artificial sequence
  synthetic polypeptide
IMEDVGWLNV

SEQ ID NO: 324 - protein, 10 aa, Artificial sequence
  synthetic polypeptide
MMWDRGLGMM

SEQ ID NO: 325 - protein, 10 aa, Artificial sequence
  synthetic polypeptide
ILEDRGFNQV

SEQ ID NO: 326 - protein, 10 aa, Artificial sequence
  synthetic polypeptide
LMFDRGMSLL

SEQ ID NO: 327 - protein, 10 aa, Artificial sequence
  synthetic polypeptide
LMLDFDGSLL

SEQ ID NO: 328 - protein, 10 aa, Artificial sequence
  synthetic polypeptide
IMEDRGSLNM

SEQ ID NO: 329 - protein, 10 aa, Artificial sequence
  synthetic polypeptide
LMNDMGFHIV

SEQ ID NO: 330 - protein, 10 aa, Artificial sequence
  synthetic polypeptide
IMEDRGSGEM

SEQ ID NO: 331 - protein, 10 aa, Artificial sequence
  synthetic polypeptide
LMWDVGLSIM

SEQ ID NO: 332 - protein, 10 aa, Artificial sequence
  synthetic polypeptide
SMWDRGTFIM

SEQ ID NO: 333 - protein, 10 aa, Artificial sequence
  synthetic polypeptide
LMLDRGSPNM

SEQ ID NO: 334 - protein, 10 aa, Artificial sequence
  synthetic polypeptide
IMFDRGIGIM

SEQ ID NO: 335 - protein, 10 aa, Artificial sequence
  synthetic polypeptide
ILFDRGMNLM

SEQ ID NO: 336 - protein, 10 aa, Artificial sequence
  synthetic polypeptide
MLLDRGLSLM

SEQ ID NO: 337 - protein, 10 aa, Artificial sequence
  synthetic polypeptide
IMEDRGSLIL

SEQ ID NO: 338 - protein, 10 aa, Artificial sequence
  synthetic polypeptide
LMRDYQLLQV

SEQ ID NO: 339 - protein, 10 aa, Artificial sequence
  synthetic polypeptide
LMFDRGMSVL

SEQ ID NO: 340 - protein, 10 aa, Artificial sequence
  synthetic polypeptide
LMEDIGRELV

SEQ ID NO: 341 - protein, 10 aa, Artificial sequence
  synthetic polypeptide
ILEDRGMGLL

SEQ ID NO: 342 - protein, 10 aa, Artificial sequence
  synthetic polypeptide
MMDQFNGLMM

SEQ ID NO: 343 - protein, 10 aa, Artificial sequence
  synthetic polypeptide
IMWDRDYGVM

SEQ ID NO: 344 - protein, 10 aa, Artificial sequence
  synthetic polypeptide
MMWDRGFNQV

SEQ ID NO: 345 - protein, 10 aa, Artificial sequence
  synthetic polypeptide
IMSMSVSNYL

SEQ ID NO: 346 - protein, 10 aa, Artificial sequence
  synthetic polypeptide
AMGDGSYLLM

SEQ ID NO: 347 - protein, 10 aa, Artificial sequence
  synthetic polypeptide
SMWDRGMGLL

SEQ ID NO: 348 - protein, 10 aa, Artificial sequence
  synthetic polypeptide
MMENRGSGAL

SEQ ID NO: 349 - protein, 10 aa, Artificial sequence
  synthetic polypeptide
LMWDSGLELM

SEQ ID NO: 350 - protein, 10 aa, Artificial sequence
  synthetic polypeptide
SMWDRGLGMM

SEQ ID NO: 351 - protein, 10 aa, Artificial sequence
  synthetic polypeptide
LMWDVGWLNV

SEQ ID NO: 352 - protein, 10 aa, Artificial sequence
  synthetic polypeptide
MMWDRGTFIM

SEQ ID NO: 353 - protein, 10 aa, Artificial sequence
  synthetic polypeptide
MMWDRGIVPV

SEQ ID NO: 354 - protein, 10 aa, Artificial sequence
  synthetic polypeptide
ILFDRGMNLM

SEQ ID NO: 355 - protein, 10 aa, Artificial sequence
  synthetic polypeptide
MLWDVQSGQM

SEQ ID NO: 356 - protein, 10 aa, Artificial sequence
  synthetic polypeptide
LLLQVGLSLL

SEQ ID NO: 357 - protein, 10 aa, Artificial sequence
  synthetic polypeptide
SLEDVVMLNV

SEQ ID NO: 358 - protein, 10 aa, Artificial sequence
  synthetic polypeptide
MLEDRDLFVM

SEQ ID NO: 359 - protein, 10 aa, Artificial sequence
  synthetic polypeptide
MLEDMSLGIM

SEQ ID NO: 360 - protein, 10 aa, Artificial sequence
  synthetic polypeptide
SLENRGLSML

SEQ ID NO: 361 - protein, 10 aa, Artificial sequence
  synthetic polypeptide
ILDDGGFLLM

SEQ ID NO: 362 - protein, 10 aa, Artificial sequence
  synthetic polypeptide
LLWNFGLLIV

SEQ ID NO: 363 - protein, 10 aa, Artificial sequence
  synthetic polypeptide
LLFDISFLML

SEQ ID NO: 364 - protein, 10 aa, Artificial sequence
  synthetic polypeptide
IMGDRNRNLL

SEQ ID NO: 365 - protein, 9 aa, Artificial sequence
  synthetic polypeptide
VMISHENFM

SEQ ID NO: 366 - protein, 9 aa, Artificial sequence
  synthetic polypeptide
TMQSHEVML

SEQ ID NO: 367 - protein, 9 aa, Artificial sequence
  synthetic polypeptide
TMQSHENFM

SEQ ID NO: 368 - protein, 9 aa, Artificial sequence
  synthetic polypeptide
VMQSHEVML

SEQ ID NO: 369 - protein, 9 aa, Artificial sequence
  synthetic polypeptide
VMISHEIFL

SEQ ID NO: 370 - protein, 9 aa, Artificial sequence
  synthetic polypeptide
IMTSHEVML

SEQ ID NO: 371 - protein, 9 aa, Artificial sequence
  synthetic polypeptide
IMTSHEVMM

SEQ ID NO: 372 - protein, 9 aa, Artificial sequence
  synthetic polypeptide
VMESHDVFM

SEQ ID NO: 373 - protein, 9 aa, Artificial sequence
  synthetic polypeptide
IMNSHEVMM

SEQ ID NO: 374 - protein, 9 aa, Artificial sequence
  synthetic polypeptide
SMNSHEVMM

SEQ ID NO: 375 - protein, 9 aa, Artificial sequence
  synthetic polypeptide
KMNSHEVMM

SEQ ID NO: 376 - protein, 9 aa, Artificial sequence
  synthetic polypeptide
AMQGHEYFL

SEQ ID NO: 377 - protein, 9 aa, Artificial sequence
  synthetic polypeptide
AMQGHEIFL

SEQ ID NO: 378 - protein, 9 aa, Artificial sequence
  synthetic polypeptide
VLQSHEVSM

SEQ ID NO: 379 - protein, 9 aa, Artificial sequence
  synthetic polypeptide
AMQSHEVTL

SEQ ID NO: 380 - protein, 9 aa, Artificial sequence
  synthetic polypeptide
LMSGDYQFV

SEQ ID NO: 381 - protein, 9 aa, Artificial sequence
  synthetic polypeptide
TMHNHEVMM

SEQ ID NO: 382 - protein, 9 aa, Artificial sequence
  synthetic polypeptide
VMHNHEVMM

SEQ ID NO: 383 - protein, 9 aa, Artificial sequence
  synthetic polypeptide
TMTGHEVFM

SEQ ID NO: 384 - protein, 9 aa, Artificial sequence
  synthetic polypeptide
TMTGHEVFV

SEQ ID NO: 385 - protein, 9 aa, Artificial sequence
  synthetic polypeptide
VMQGHESFL

SEQ ID NO: 386 - protein, 9 aa, Artificial sequence
  synthetic polypeptide
VMISHEVML

SEQ ID NO: 387 - protein, 9 aa, Artificial sequence
  synthetic polypeptide
TMTGHEVML

SEQ ID NO: 388 - protein, 9 aa, Artificial sequence
  synthetic polypeptide
SMVGMEHSM

SEQ ID NO: 389 - protein, 9 aa, Artificial sequence
  synthetic polypeptide
AMQGHEHFM

SEQ ID NO: 390 - protein, 9 aa, Artificial sequence
  synthetic polypeptide
VMEGDYWFL

SEQ ID NO: 391 - protein, 9 aa, Artificial sequence
  synthetic polypeptide
SMQSHEWML

SEQ ID NO: 392 - protein, 9 aa, Artificial sequence
  synthetic polypeptide
YMQTHESFM

SEQ ID NO: 393 - protein, 10 aa, Artificial sequence
  synthetic polypeptide
VMNGDSGTFL

SEQ ID NO: 394 - protein, 10 aa, Artificial sequence
  synthetic polypeptide
YMAVRSENFM

SEQ ID NO: 395 - protein, 10 aa, Artificial sequence
  synthetic polypeptide
RMPNKQENFV

SEQ ID NO: 396 - protein, 10 aa, Artificial sequence
  synthetic polypeptide
IMDSKSEHFM

SEQ ID NO: 397 - protein, 10 aa, Artificial sequence
  synthetic polypeptide
IMDSREEVFV

SEQ ID NO: 398 - protein, 10 aa, Artificial sequence
  synthetic polypeptide
IMDSRSEHFM

SEQ ID NO: 399 - protein, 10 aa, Artificial sequence
  synthetic polypeptide
GMDSRAEVFM

SEQ ID NO: 400 - protein, 10 aa, Artificial sequence
  synthetic polypeptide
ALDSRSEYFL

SEQ ID NO: 401 - protein, 10 aa, Artificial sequence
  synthetic polypeptide
KMANRDENFV

SEQ ID NO: 402 - protein, 10 aa, Artificial sequence
  synthetic polypeptide
RLDGQDTKFM

SEQ ID NO: 403 - protein, 10 aa, Artificial sequence
  synthetic polypeptide
LMDSRSEHFM

SEQ ID NO: 404 - protein, 10 aa, Artificial sequence
  synthetic polypeptide
IMNSRSELFL

SEQ ID NO: 405 - protein, 10 aa, Artificial sequence
  synthetic polypeptide
MMNVRSELFV

SEQ ID NO: 406 - protein, 10 aa, Artificial sequence
  synthetic polypeptide
TMNVRSELFV

SEQ ID NO: 407 - protein, 10 aa, Artificial sequence
  synthetic polypeptide
KMNSRSELFL

SEQ ID NO: 408 - protein, 10 aa, Artificial sequence
  synthetic polypeptide
TMNVRSEHFM

SEQ ID NO: 409 - protein, 10 aa, Artificial sequence
  synthetic polypeptide
SMNSRSELFL

SEQ ID NO: 410 - protein, 10 aa, Artificial sequence
  synthetic polypeptide
KMNSRSEHFM

SEQ ID NO: 411 - protein, 10 aa, Artificial sequence
  synthetic polypeptide
TMQSHDASFL

SEQ ID NO: 412 - protein, 10 aa, Artificial sequence
  synthetic polypeptide
VMQGHDASFL

SEQ ID NO: 413 - protein, 10 aa, Artificial sequence
  synthetic polypeptide
KMNSHSGTFL

SEQ ID NO: 414 - protein, 10 aa, Artificial sequence
  synthetic polypeptide
KMNGKSEDFM

SEQ ID NO: 415 - protein, 10 aa, Artificial sequence
  synthetic polypeptide
DMDNRLDRDM

SEQ ID NO: 416 - protein, 10 aa, Artificial sequence
  synthetic polypeptide
IMDSKSEIFL

SEQ ID NO: 417 - protein, 10 aa, Artificial sequence
  synthetic polypeptide
SMNSHSGTFL

SEQ ID NO: 418 - protein, 10 aa, Artificial sequence
  synthetic polypeptide
SMNSREEHFM

SEQ ID NO: 419 - protein, 10 aa, Artificial sequence
  synthetic polypeptide
IMNSHSGTFL

SEQ ID NO: 420 - protein, 10 aa, Artificial sequence
  synthetic polypeptide
IMDSKSENFL

SEQ ID NO: 421 - protein, 10 aa, Artificial sequence
  synthetic polypeptide
AMDSKSENFL

SEQ ID NO: 422 - protein, 10 aa, Artificial sequence
  synthetic polypeptide
IMDSRADMFV

SEQ ID NO: 423 - protein, 10 aa, Artificial sequence
  synthetic polypeptide
SMNSREEVFV

SEQ ID NO: 424 - protein, 10 aa, Artificial sequence
  synthetic polypeptide
KMNSREEVFV

SEQ ID NO: 425 - protein, 10 aa, Artificial sequence
  synthetic polypeptide
ALDSRSEHFM

SEQ ID NO: 426 - protein, 10 aa, Artificial sequence
  synthetic polypeptide
AMDSRSEHFM

SEQ ID NO: 427 - protein, 10 aa, Artificial sequence
  synthetic polypeptide
AMDSRADMFV

SEQ ID NO: 428 - protein, 10 aa, Artificial sequence
  synthetic polypeptide
LMDSRSQIFV

SEQ ID NO: 429 - protein, 10 aa, Artificial sequence
  synthetic polypeptide
GMTSRSDYMV

SEQ ID NO: 430 - protein, 10 aa, Artificial sequence
  synthetic polypeptide
VMNSRSEHFM

SEQ ID NO: 431 - protein, 10 aa, Artificial sequence
  synthetic polypeptide
VMNSRSDWFL

SEQ ID NO: 432 - protein, 10 aa, Artificial sequence
  synthetic polypeptide
YMNSHDPYTV

SEQ ID NO: 433 - protein, 10 aa, Artificial sequence
  synthetic polypeptide
RMDSRSQDFV

SEQ ID NO: 434 - protein, 10 aa, Artificial sequence
  synthetic polypeptide
RMEAHSSHFV

SEQ ID NO: 435 - protein, 10 aa, Artificial sequence
  synthetic polypeptide
TLMSRSDLFL

SEQ ID NO: 436 - protein, 10 aa, Artificial sequence
  synthetic polypeptide
ILNSRDEAMM

SEQ ID NO: 437 - protein, 10 aa, Artificial sequence
  synthetic polypeptide
ALNSRDEAMM

SEQ ID NO: 438 - protein, 10 aa, Artificial sequence
  synthetic polypeptide
ALDSRLEFFV

SEQ ID NO: 439 - protein, 10 aa, Artificial sequence
  synthetic polypeptide
VMDSRLEFFV

SEQ ID NO: 440 - protein, 10 aa, Artificial sequence
  synthetic polypeptide
ALDSRSELFL

SEQ ID NO: 441 - protein, 10 aa, Artificial sequence
  synthetic polypeptide
AMYSNSDFMV

SEQ ID NO: 442 - protein, 10 aa, Artificial sequence
  synthetic polypeptide
VMDSRLEHFM

SEQ ID NO: 443 - protein, 10 aa, Artificial sequence
  synthetic polypeptide
SMNSRSEHFM

SEQ ID NO: 444 - protein, 10 aa, Artificial sequence
  synthetic polypeptide
SMNSKSENFL

SEQ ID NO: 445 - protein, 10 aa, Artificial sequence
  synthetic polypeptide
VLDSSSSSFL

SEQ ID NO: 446 - protein, 10 aa, Artificial sequence
  synthetic polypeptide
ALDSRSENFL

SEQ ID NO: 447 - protein, 10 aa, Artificial sequence
  synthetic polypeptide
ALDSKSENFL

SEQ ID NO: 448 - protein, 10 aa, Artificial sequence
  synthetic polypeptide
ALDSRSEIFL

SEQ ID NO: 449 - protein, 10 aa, Artificial sequence
  synthetic polypeptide
SMNSRADMFV

SEQ ID NO: 450 - protein, 10 aa, Artificial sequence
  synthetic polypeptide
SMYSRQEMMV

SEQ ID NO: 451 - protein, 10 aa, Artificial sequence
  synthetic polypeptide
RMWSRSEDMV

SEQ ID NO: 452 - protein, 10 aa, Artificial sequence
  synthetic polypeptide
VLRARSDVFV

SEQ ID NO: 453 - protein, 10 aa, Artificial sequence
  synthetic polypeptide
ALDSREEVFV

SEQ ID NO: 454 - protein, 10 aa, Artificial sequence
  synthetic polypeptide
SMNSREEIFL

SEQ ID NO: 455 - protein, 10 aa, Artificial sequence
  synthetic polypeptide
SMSGFSESFV

SEQ ID NO: 456 - protein, 11 aa, Artificial sequence
  synthetic polypeptide
ILSNRGHEVFV

SEQ ID NO: 457 - protein, 11 aa, Artificial sequence
  synthetic polypeptide
ILSNRGHENFM

SEQ ID NO: 458 - protein, 11 aa, Artificial sequence
  synthetic polypeptide
ILSNRGHDVFM

SEQ ID NO: 459 - protein, 11 aa, Artificial sequence
  synthetic polypeptide
ILSNRGHEIFL

SEQ ID NO: 460 - protein, 11 aa, Artificial sequence
  synthetic polypeptide
ILSNRGHEYFL

SEQ ID NO: 461 - protein, 11 aa, Artificial sequence
  synthetic polypeptide
CASSLGLEQFF

SEQ ID NO: 462 - protein, 14 aa, Artificial sequence
  synthetic polypeptide
CASSLGGGHTEAFF

SEQ ID NO: 463 - protein, 13 aa, Artificial sequence
  synthetic polypeptide
CASSLVNGLGYTF

SEQ ID NO: 464 - protein, 15 aa, Artificial sequence
  synthetic polypeptide
CATSRDRGQDEKLFF

SEQ ID NO: 465 - protein, 15 aa, Artificial sequence
  synthetic polypeptide
CASSADTGVNQPQHF

SEQ ID NO: 466 - protein, 14 aa, Artificial sequence
  synthetic polypeptide
CASSRDTVNTEAFF

SEQ ID NO: 467 - protein, 14 aa, Artificial sequence
  synthetic polypeptide
CSARDYQGSQPQHF

SEQ ID NO: 468 - protein, 14 aa, Artificial sequence
  synthetic polypeptide
CSARDYQGSQPQHF

SEQ ID NO: 469 - protein, 15 aa, Artificial sequence
  synthetic polypeptide
CASSADTGVNQPQHF

SEQ ID NO: 470 - protein, 12 aa, Artificial sequence
  synthetic polypeptide
CAGGGGADGLTF

SEQ ID NO: 471 - protein, 15 aa, Artificial sequence
  synthetic polypeptide
CALSEAEAAGNKLTF

SEQ ID NO: 472 - protein, 16 aa, Artificial sequence
  synthetic polypeptide
CALSEAGMDSNYQLIW

SEQ ID NO: 473 - protein, 18 aa, Artificial sequence
  synthetic polypeptide
CAMREGRYSGAGSYQLTF

SEQ ID NO: 474 - protein, 13 aa, Artificial sequence
  synthetic polypeptide
CVVTETNAGKSTF

SEQ ID NO: 475 - protein, 16 aa, Artificial sequence
  synthetic polypeptide
CALSEARGGATNKLIF

SEQ ID NO: 476 - protein, 13 aa, Artificial sequence
  synthetic polypeptide
CAVNSGNTGKLIF

SEQ ID NO: 477 - protein, 15 aa, Artificial sequence
  synthetic polypeptide
CAVPFLYNQGGKLIF

SEQ ID NO: 478 - protein, 11 aa, Artificial sequence
  synthetic polypeptide
CAVNDFNKFYF

SEQ ID NO: 479 - protein, 16 aa, Artificial sequence
  synthetic polypeptide
CASSQGVGQFKNTQYF

SEQ ID NO: 480 - protein, 17 aa, Artificial sequence
  synthetic polypeptide
CASSLSGRQGGSYEQYF

SEQ ID NO: 481 - protein, 15 aa, Artificial sequence
  synthetic polypeptide
CASSSSGGLVDTQYF

SEQ ID NO: 482 - protein, 12 aa, Artificial sequence
  synthetic polypeptide
CASMGRSYGYTF

SEQ ID NO: 483 - protein, 16 aa, Artificial sequence
  synthetic polypeptide
CASSLETGTAIYEQYF

SEQ ID NO: 484 - protein, 19 aa, Artificial sequence
  synthetic polypeptide
CASSPSGLAGSNLGNEQFF

SEQ ID NO: 485 - protein, 14 aa, Artificial sequence
  synthetic polypeptide
CASSRIDSTDTQYF

SEQ ID NO: 486 - protein, 15 aa, Artificial sequence
  synthetic polypeptide
CASSIPRGSSQPQHF

SEQ ID NO: 487 - protein, 16 aa, Artificial sequence
  synthetic polypeptide
CAIKGGDRGVNTEAFF

SEQ ID NO: 488 - protein, 13 aa, Artificial sequence
  synthetic polypeptide
CSARLASYNEQFF

SEQ ID NO: 489 - protein, 14 aa, Artificial sequence
  synthetic polypeptide
CASSRDFVSNEQYF

SEQ ID NO: 490 - protein, 13 aa, Artificial sequence
  synthetic polypeptide
CAVETSNTGKLIF

SEQ ID NO: 491 - protein, 12 aa, Artificial sequence
  synthetic polypeptide
CAASSTGNQFYF

SEQ ID NO: 492 - protein, 17 aa, Artificial sequence
  synthetic polypeptide
CALSAGASGAGSYQLTF

SEQ ID NO: 493 - protein, 15 aa, Artificial sequence
  synthetic polypeptide
CALMNYGGATNKLIF

SEQ ID NO: 494 - protein, 12 aa, Artificial sequence
  synthetic polypeptide
CAADNNNARLMF

SEQ ID NO: 495 - protein, 15 aa, Artificial sequence
  synthetic polypeptide
CALSSRGSTLGRLYF

SEQ ID NO: 496 - protein, 14 aa, Artificial sequence
  synthetic polypeptide
CLVGEVGTASKLTF

SEQ ID NO: 497 - protein, 13 aa, Artificial sequence
  synthetic polypeptide
CAVDSGGYNKLIF

SEQ ID NO: 498 - protein, 15 aa, Artificial sequence
  synthetic polypeptide
CAMREPNNAGNMLTF

SEQ ID NO: 499 - protein, 15 aa, Artificial sequence
  synthetic polypeptide
CAVRRATDSWGKLQF

SEQ ID NO: 500 - protein, 16 aa, Artificial sequence
  synthetic polypeptide
CALSEARGGATNKLIF

SEQ ID NO: 501 - protein, 10 aa, Artificial sequence
  synthetic polypeptide
YLAPQESYGA

SEQ ID NO: 502 - protein, 10 aa, Artificial sequence
  synthetic polypeptide
YASSYIILAM

SEQ ID NO: 503 - protein, 10 aa, Artificial sequence
  synthetic polypeptide
VMLQIINIVL

SEQ ID NO: 504 - protein, 10 aa, Artificial sequence
  synthetic polypeptide
VLSWLLKYKI

SEQ ID NO: 505 - protein, 10 aa, Artificial sequence
  synthetic polypeptide
SVLNYFKPYL

SEQ ID NO: 506 - protein, 10 aa, Artificial sequence
  synthetic polypeptide
SLMTPNTITM

SEQ ID NO: 507 - protein, 10 aa, Artificial sequence
  synthetic polypeptide
RVLSHDSIFI

SEQ ID NO: 508 - protein, 10 aa, Artificial sequence
  synthetic polypeptide
NLNPNVDPQV

SEQ ID NO: 509 - protein, 10 aa, Artificial sequence
  synthetic polypeptide
LLQEEAHVPL

SEQ ID NO: 510 - protein, 10 aa, Artificial sequence
  synthetic polypeptide
LIYELYVSEL

SEQ ID NO: 511 - protein, 10 aa, Artificial sequence
  synthetic polypeptide
KTYIIFFVLV

SEQ ID NO: 512 - protein, 10 aa, Artificial sequence
  synthetic polypeptide
KLYGLDWAEL

SEQ ID NO: 513 - protein, 10 aa, Artificial sequence
  synthetic polypeptide
KLFEFLVYGV

SEQ ID NO: 514 - protein, 10 aa, Artificial sequence
  synthetic polypeptide
IVAADLIMTL

SEQ ID NO: 515 - protein, 10 aa, Artificial sequence
  synthetic polypeptide
IQYLELNRLV

SEQ ID NO: 516 - protein, 10 aa, Artificial sequence
  synthetic polypeptide
IQVWEALLTL

SEQ ID NO: 517 - protein, 10 aa, Artificial sequence
  synthetic polypeptide
ILSGGRTLQI

SEQ ID NO: 518 - protein, 10 aa, Artificial sequence
  synthetic polypeptide
HVMLQIINIV

SEQ ID NO: 519 - protein, 10 aa, Artificial sequence
  synthetic polypeptide
HMMGFRTQEV

SEQ ID NO: 520 - protein, 10 aa, Artificial sequence
  synthetic polypeptide
HIYIGIHMCV

SEQ ID NO: 521 - protein, 10 aa, Artificial sequence
  synthetic polypeptide
GMYASSYIIL

SEQ ID NO: 522 - protein, 10 aa, Artificial sequence
  synthetic polypeptide
GLLPVLSWLL

SEQ ID NO: 523 - protein, 10 aa, Artificial sequence
  synthetic polypeptide
FNQLIYELYV

SEQ ID NO: 524 - protein, 10 aa, Artificial sequence
  synthetic polypeptide
FMTKINDLEV

SEQ ID NO: 525 - protein, 10 aa, Artificial sequence
  synthetic polypeptide
FLVYGVRPGM

SEQ ID NO: 526 - protein, 10 aa, Artificial sequence
  synthetic polypeptide
FLPVTDASSV

SEQ ID NO: 527 - protein, 10 aa, Artificial sequence
  synthetic polypeptide
FALLQEEAHV

SEQ ID NO: 528 - protein, 10 aa, Artificial sequence
  synthetic polypeptide
FALGNVISAL

SEQ ID NO: 529 - protein, 10 aa, Artificial sequence
  synthetic polypeptide
DLSYTWNIPV

SEQ ID NO: 530 - protein, 10 aa, Artificial sequence
  synthetic polypeptide
AVFYTILTPV

SEQ ID NO: 531 - protein, 10 aa, Artificial sequence
  synthetic polypeptide
ATLDWSKNAV

SEQ ID NO: 532 - protein, 10 aa, Artificial sequence
  synthetic polypeptide
ASMTGIVYSL

SEQ ID NO: 533 - protein, 10 aa, Artificial sequence
  synthetic polypeptide
ALLETPSLLL

SEQ ID NO: 534 - protein, 10 aa, Artificial sequence
  synthetic polypeptide
ALDPHSGHFV

SEQ ID NO: 535 - protein, 10 aa, Artificial sequence
  synthetic polypeptide
ALAFTPVEQV

SEQ ID NO: 536 - DNA, 48 bp, Artificial sequence
  synthetic nucleotide
tgtgctctgagtgaggcgaggggtggtgctacaaacaagctcatcttt

SEQ ID NO: 537 - DNA, 48 bp, Artificial sequence
  synthetic nucleotide
tgtgctctgagtgaggcgcggggcggtgctacaaacaagctcatcttt

SEQ ID NO: 538 - DNA, 42 bp, Artificial sequence
  synthetic nucleotide
tgcgccagcagccgggacactgttaatactgaagctttcttt

SEQ ID NO: 539 - DNA, 42 bp, Artificial sequence
  synthetic nucleotide
tgcgccagcagtcgggacttcgtgtccaacgagcagtacttc

SEQ ID NO: 540 - protein, 8 aa, Artificial sequence
  synthetic polypeptide
SMGVTYEM

SEQ ID NO: 541 - protein, 8 aa, Artificial sequence
  synthetic polypeptide
YMGVSYEM

SEQ ID NO: 542 - protein, 8 aa, Artificial sequence
  synthetic polypeptide
YMGVVYEM

SEQ ID NO: 543 - protein, 8 aa, Artificial sequence
  synthetic polypeptide
KMGVTYEM

SEQ ID NO: 544 - protein, 8 aa, Artificial sequence
  synthetic polypeptide
FMGVTYEM

SEQ ID NO: 545 - protein, 8 aa, Artificial sequence
  synthetic polypeptide
NMEVTYEI

SEQ ID NO: 546 - protein, 8 aa, Artificial sequence
  synthetic polypeptide
FITVTEEI

SEQ ID NO: 547 - protein, 8 aa, Artificial sequence
  synthetic polypeptide
HIQVTNEI

SEQ ID NO: 548 - protein, 8 aa, Artificial sequence
  synthetic polypeptide
HLIVSYEL

SEQ ID NO: 549 - protein, 8 aa, Artificial sequence
  synthetic polypeptide
HLGVTKEL

SEQ ID NO: 550 - protein, 8 aa, Artificial sequence
  synthetic polypeptide
RLGVTYFV

SEQ ID NO: 551 - protein, 8 aa, Artificial sequence
  synthetic polypeptide
YLPVTYHI

SEQ ID NO: 552 - protein, 8 aa, Artificial sequence
  synthetic polypeptide
GLGQTYEI

SEQ ID NO: 553 - protein, 8 aa, Artificial sequence
  synthetic polypeptide
EYGVSYEW

SEQ ID NO: 554 - protein, 8 aa, Artificial sequence
  synthetic polypeptide
EYGVQNYV

SEQ ID NO: 555 - protein, 8 aa, Artificial sequence
  synthetic polypeptide
EMGVSYEM

SEQ ID NO: 556 - protein, 9 aa, Artificial sequence
  synthetic polypeptide
LMDMHNGQL

SEQ ID NO: 557 - protein, 9 aa, Artificial sequence
  synthetic polypeptide
RLDAMNGQL

SEQ ID NO: 558 - protein, 9 aa, Artificial sequence
  synthetic polypeptide
RMDYNNMQM

SEQ ID NO: 559 - protein, 9 aa, Artificial sequence
  synthetic polypeptide
SMDTFQGQM

SEQ ID NO: 560 - protein, 9 aa, Artificial sequence
  synthetic polypeptide
GMDYHNGHL

SEQ ID NO: 561 - protein, 9 aa, Artificial sequence
  synthetic polypeptide
TMDFYQGQL

SEQ ID NO: 562 - protein, 9 aa, Artificial sequence
  synthetic polypeptide
KMDYFSGQL

SEQ ID NO: 563 - protein, 9 aa, Artificial sequence
  synthetic polypeptide
SMDWFQGQM

SEQ ID NO: 564 - protein, 9 aa, Artificial sequence
  synthetic polypeptide
LMDYWQGQL

SEQ ID NO: 565 - protein, 9 aa, Artificial sequence
  synthetic polypeptide
NMMWFQGQL

SEQ ID NO: 566 - protein, 9 aa, Artificial sequence
  synthetic polypeptide
VLDLFQGQL

SEQ ID NO: 567 - protein, 9 aa, Artificial sequence
  synthetic polypeptide
MMDFFNAQM

SEQ ID NO: 568 - protein, 9 aa, Artificial sequence
  synthetic polypeptide
LLNLNNGQL

SEQ ID NO: 569 - protein, 9 aa, Artificial sequence
  synthetic polypeptide
QMDYEEGQL

SEQ ID NO: 570 - protein, 9 aa, Artificial sequence
  synthetic polypeptide
GLSSQNGQL

SEQ ID NO: 571 - protein, 9 aa, Artificial sequence
  synthetic polypeptide
TLHYYEMHL

SEQ ID NO: 572 - protein, 9 aa, Artificial sequence
  synthetic polypeptide
VIDFLNNQL

SEQ ID NO: 573 - protein, 9 aa, Artificial sequence
  synthetic polypeptide
VIDQLNGQL

SEQ ID NO: 574 - protein, 9 aa, Artificial sequence
  synthetic polypeptide
VVDFLKGQL

SEQ ID NO: 575 - protein, 9 aa, Artificial sequence
  synthetic polypeptide
LMDMHNGQL

SEQ ID NO: 576 - protein, 9 aa, Artificial sequence
  synthetic polypeptide
RLDAMNGQL

SEQ ID NO: 577 - protein, 9 aa, Artificial sequence
  synthetic polypeptide
RMDYNNMQM

SEQ ID NO: 578 - protein, 9 aa, Artificial sequence
  synthetic polypeptide
SMDTFQGQM

SEQ ID NO: 579 - protein, 9 aa, Artificial sequence
  synthetic polypeptide
GMDYHNGHL

SEQ ID NO: 580 - protein, 9 aa, Artificial sequence
  synthetic polypeptide
TMDFYQGQL

SEQ ID NO: 581 - protein, 9 aa, Artificial sequence
  synthetic polypeptide
KMDYFSGQL

SEQ ID NO: 582 - protein, 9 aa, Artificial sequence
  synthetic polypeptide
SMDWFQGQM

SEQ ID NO: 583 - protein, 9 aa, Artificial sequence
  synthetic polypeptide
LMDYWQGQL

SEQ ID NO: 584 - protein, 9 aa, Artificial sequence
  synthetic polypeptide
NMMWFQGQL

SEQ ID NO: 585 - protein, 9 aa, Artificial sequence
  synthetic polypeptide
VLDLFQGQL

SEQ ID NO: 586 - protein, 9 aa, Artificial sequence
  synthetic polypeptide
MMDFFNAQM

SEQ ID NO: 587 - protein, 9 aa, Artificial sequence
  synthetic polypeptide
LLNLNNGQL

SEQ ID NO: 588 - protein, 9 aa, Artificial sequence
  synthetic polypeptide
MMDFFNAQM

SEQ ID NO: 589 - protein, 9 aa, Artificial sequence
  synthetic polypeptide
LLNLNNGQL

SEQ ID NO: 590 - protein, 9 aa, Artificial sequence
  synthetic polypeptide
QMDYEEGQL

SEQ ID NO: 591 - protein, 9 aa, Artificial sequence
  synthetic polypeptide
GLSSQNGQL

SEQ ID NO: 592 - protein, 9 aa, Artificial sequence
  synthetic polypeptide
TLHYYEMHL

SEQ ID NO: 593 - protein, 9 aa, Artificial sequence
  synthetic polypeptide
VIDFLNNQL

SEQ ID NO: 594 - protein, 9 aa, Artificial sequence
  synthetic polypeptide
VIDQLNGQL

SEQ ID NO: 595 - protein, 9 aa, Artificial sequence
  synthetic polypeptide
VVDFLKGQL

SEQ ID NO: 596 - protein, 9 aa, Artificial sequence
  synthetic polypeptide
RMEQVDWTV

SEQ ID NO: 597 - protein, 9 aa, Artificial sequence
  synthetic polypeptide
KLEFMDWRL

SEQ ID NO: 598 - protein, 9 aa, Artificial sequence
  synthetic polypeptide
WLDNFELCL

SEQ ID NO: 599 - protein, 9 aa, Artificial sequence
  synthetic polypeptide
TLEYMDWLV

SEQ ID NO: 600 - protein, 9 aa, Artificial sequence
  synthetic polypeptide
EMMLFDWKV

SEQ ID NO: 601 - protein, 9 aa, Artificial sequence
  synthetic polypeptide
KLEQLDWTV

SEQ ID NO: 602 - protein, 9 aa, Artificial sequence
  synthetic polypeptide
TMETIDWKV

SEQ ID NO: 603 - protein, 9 aa, Artificial sequence
  synthetic polypeptide
DLEQMEQTV

SEQ ID NO: 604 - protein, 9 aa, Artificial sequence
  synthetic polypeptide
TLEELDWCL

SEQ ID NO: 605 - protein, 9 aa, Artificial sequence
  synthetic polypeptide
TLEDMAWRL

SEQ ID NO: 606 - protein, 9 aa, Artificial sequence
  synthetic polypeptide
NVEEMDWLI

SEQ ID NO: 607 - protein, 9 aa, Artificial sequence
  synthetic polypeptide
NVEEMDWMV

SEQ ID NO: 608 - protein, 9 aa, Artificial sequence
  synthetic polypeptide
LLEDLDWDV

SEQ ID NO: 609 - protein, 9 aa, Artificial sequence
  synthetic polypeptide
TLEAMNTTV

SEQ ID NO: 610 - protein, 9 aa, Artificial sequence
  synthetic polypeptide
VLEEVDWLI

SEQ ID NO: 611 - protein, 9 aa, Artificial sequence
  synthetic polypeptide
WLEDVEWQV

SEQ ID NO: 612 - protein, 9 aa, Artificial sequence
  synthetic polypeptide
KMENFDKTV

SEQ ID NO: 613 - protein, 9 aa, Artificial sequence
  synthetic polypeptide
NMEYMTWDV

SEQ ID NO: 614 - protein, 9 aa, Artificial sequence
  synthetic polypeptide
FVENVEWRV

SEQ ID NO: 615 - protein, 9 aa, Artificial sequence
  synthetic polypeptide
NVEYYDIKL

SEQ ID NO: 616 - protein, 9 aa, Artificial sequence
  synthetic polypeptide
HLEQVDKAV

SEQ ID NO: 617 - protein, 9 aa, Artificial sequence
  synthetic polypeptide
EMEQVDAVV

SEQ ID NO: 618 - protein, 9 aa, Artificial sequence
  synthetic polypeptide
SMEQFTVRV

SEQ ID NO: 619 - protein, 9 aa, Artificial sequence
  synthetic polypeptide
HMNNVTVTL

SEQ ID NO: 620 - protein, 10 aa, Artificial sequence
  synthetic polypeptide
WLIDMKSLVM

(19) United States
(12) Patent Application Publication
(10) Pub. No.: US 2020/0010527 A1
(43) Pub. Date: Jan. 9, 2020

(54) Antigen Discovery for T Cell Receptors Isolated from Patient Tumors Recognizing Wild-Type Antigens and Potent Peptide Mimotopes

(71) Applicant: The Board of Trustees of the Leland Stanford Junior University, Stanford, CA (US)

(72) Inventors: Marvin Gee, Palo Alto, CA (US); Mark M. Davis, Atherton, CA (US); Arnold Han, Los Altos Hills, CA (US); Kenan Christopher Garcia, Menlo Park, CA (US)

(21) Appl. No.: 16/492,898

(22) PCT Filed: Mar. 21, 2018
(86) PCT No.: PCT/US2018/023569

§ 371 Date: Sep. 10, 2019

Related U.S. Application Data
(60) Provisional application No. 62/476,575, filed on Mar. 24, 2017.

(51) Int. Cl. C07K 14/725; G01N 33/50; A61K 39/00; C07K 14/74; G01N 33/574

(52) U.S. Cl. CPC C07K 14/7051; G01N 33/505; A61K 39/00; C07K 14/70539; A61K 2039/5158; C07K 2319/21; C07K 2319/50; G01N 2800/52; G01N 33/57492

(57) ABSTRACT

Compositions and methods are provided for peptide sequences that are ligands for a T cell receptor (TCR) of interest, in a given MHC context.

### CROSS REFERENCE

This application claims benefit of U.S. Provisional Patent Application No. 62/476,575, filed Mar. 24, 2017 which application is incorporated herein by reference in its entirety.

### BACKGROUND

T cells are integral to the adaptive immune system and provide protection against pathogens and cancer. They function through extracellular recognition by the TCR, which is specific for short peptides presented on the human leukocyte antigen (HLA) on cells (Birnbaum et al., (2014) Cell 157, 1073-1087). The diversities inherent to the TCR, peptide, and HLA molecules make identifying the specificity of any one TCR an extremely complex problem. While our ability to characterize T cells and sequence their TCRs has recently improved considerably (Han et al., (2014) Nat Biotechnol 32, 684-692; Stubbington et al., (2016) Nat Methods 13, 329-332), the ability to determine and study the antigen specificities of T cells has not similarly advanced.

Each human individual has 10¹² T cells in their body with 10⁷ to 10⁸ unique T cell receptors. Each T cell expresses a unique T cell receptor (TCR), selected for the ability to bind to major histocompatibility complex (MHC) molecules presenting peptides. TCR recognition of peptide-MHC (μMHC) drives T cell development, survival, and effector functions. Even though TCR ligands are relatively low affinity (1-100 μM), the TCRs are remarkably sensitive, requiring as few as 10 agonist peptides to fully activate a T cell. After recognition, a signaling cascade allows T cells to carry out their immune functions.

Extensive structural studies of TCR recognition of μMHC show the vast majority of studied TCR-μMHC complexes share a consistent binding orientation, driven by conserved contacts between the tops of the MHC helices and the germline-encoded TCR CDR1 and CDR2 loops (see Garcia and Adams (2005) Cell 122, 333-336; Garcia et al. (2009) Nat Immunol 10, 143-147; and Rudolph et al. (2006) Annual Review of Immunology 24, 419-466). These conserved contacts have likely coevolved throughout the development of the adaptive immune system and serve as the basis of MHC restriction of the aβ TCR repertoire (Scott-Browne et al., 2011). Alteration to the typical TCR-μMHC interaction has been shown to correlate with abrogated signaling and, when present in development, skewed TCR repertoires (Adams et al. (2011) Immunity 35(5):681-93; Birnbaum et al. (2012) Immunol. Rev. 250(1):82-101).

An additional important feature of the TCR is the ability to balance cross-reactivity with specificity. Since the number of T cells that would be necessary to uniquely recognize every possible μMHC combination is extremely high, and since there are few if any ‘holes’ characterized in the TCR repertoire, it has been posited that a large degree of TCR cross-reactivity is a requirement of functional antigen recognition. How the T cell repertoire can simultaneously be MHC restricted, cross-reactive enough to ensure all potential antigenic challenges can be met, yet still specific enough to avoid aberrant autoimmunity, has remained an open and pressing question in immunology.

There have been a number of strategies used to determine the specificity of orphan TCRs (Birnbaum et al., (2012) Immunol Rev 250, 82-101). Mass spectrometry can provide an unbiased method of antigen isolation, but is restricted to experiments requiring large cell numbers, typically 10⁷ to 10⁹, and the targets must still be presented by the correct HLA. Traditionally, most studies of T cell antigen specificities have involved testing candidate antigens empirically. For example, studies of anti-tumor T cell specificities have correctly postulated that there are productive T cell responses towards neo-antigens. Such studies involve sequencing of tumors to identify mutations, using epitope prediction algorithms to predict immunogenic mutant peptides, and testing for T cell responses directed at these mutant peptides (Kreiter et al., (2015) Nature 520, 692-696; Rajasagi et al., (2014) Blood 124, 453-462; Tran et al., (2014) Science 344, 641-645). Other strategies query established T cell specificities in patients by using pHLA multimers (Bentzen et al., (2016) Nat Biotechnol 34, 1037-1045; Newell et al., (2013) Nat Biotechnol 31, 623-629).

High-throughput and sensitive approaches to determining the specificity of ‘orphan’ TCRs (i.e. TCRs of unknown antigen specificity) that could help uncover potential targets for cancer immunotherapy, autoimmunity, and infection and provide mechanistic insight into disease pathogenesis are of great interest.

### SUMMARY

Compositions are provided for ligands for a T cell receptor (TCR) of interest in a defined MHC context. The composition may comprise or consist of a defined peptide, or may comprise or consist of a polynucleotide encoding such a peptide. Such peptides may be fragments of naturally occurring antigenic proteins; may be fragments of neoantigenic proteins that are the subject of somatic mutation during tumorigenesis, or may be a synthetically generated mimic of an antigenic protein. The synthetic peptides can act as highly potent agonists of T cell receptors. In some embodiments a peptide, or encoding sequence, is selected from sequences provided herein, including without limitation any one or a combination of the peptide sequences set forth in SEQ ID NO:1-257. A peptide may be provided as short antigenic sequence active in stimulating T cells; or may be provided in the form of the larger protein, e.g. an intact domain, a soluble protein portion, a complete protein, etc. In some embodiments, peptide antigens are identified that are shared between patients and provide a means for broadly applicable therapy. In other embodiments identification of antigens provides for a personalized medicine approach.

Identification of T cell receptors and cognate antigens provides targets for immunotherapy, including screening of patient T cells for responsiveness, vaccination with peptides or nucleic acids encoding such peptides, cell-based therapies, protein-based therapies, etc. The peptides and methods disclosed herein are useful in classifying TCRs based on peptide antigen specificities, which allows the identification of clinical candidate TCRs that recognize shared antigens across patients.

In some embodiments, methods are provided for vaccination against cancer, for example colorectal cancer, the method comprising administering an effective dose of a vaccine composition, which composition may comprise a peptide identified herein; a combination of peptides, e.g. 2, 3, 4, 5, 6, 7, 8, 9, 10 or more distinct peptides; a complex of a peptide and at least a portion of an MHC protein; an autologous or allogeneic T cell that has been stimulated to respond to an antigenic peptide identified herein; a nucleic acid encoding an antigenic peptide identified herein; and optionally a pharmaceutically acceptable excipient, which may comprise a vaccine adjuvant. The peptide vaccination strategy may be used to initially prime an immune response, e.g. with a synthetic peptide provided herein, followed by a boost with the corresponding known wildtype antigen or wildtype whole protein.

The defined peptides are identified by screening peptide-MHC libraries by yeast-display was used to identify the recognition landscape of individual T cell receptors. The screening method may be utilized in a multiplex method to screen a plurality of peptide libraries simultaneously, e.g. screening 2, 3, 4 or more libraries simultaneously. Multiplexing allows improved efficiency of antigen discovery. Each library may comprise a unique epitope tag, e.g. an epitope targetable by an antibody, to allow identification; may comprise DNA barcodes; protein barcodes; etc. Each library utilizing the epitope tags were generated separately and diversities calculated, e.g. based on colony counts from limiting dilution of the initial libraries on growth plates. Pooling T cell receptors for library selection can further multiplex the selection, e.g. multiplexing of peptide sequence, peptide lengths, collections of different MHC or HLA alleles, etc. For selections, each barcode, epitope tag, etc. may be monitored via anti-epitope tag staining to detect the level of peptide-specific enrichment. statistical algorithms and machine-learning algorithms may be used for identification.

In some embodiments sequences of T cell receptors responsive to cancer antigens are provided. T cell receptor sequences may include, without limitation, the proteins having an alpha chain with sequence set forth in SEQ ID NO:258, optionally combined with a beta chain sequence of SEQ ID NO:259 or SEQ ID NO:260. The binding regions (CDR) sequences of these T cell receptors may be grafted onto an antibody framework to provide a TCR-like antibody. Because T cell receptors are adaptable and often unique from patient-to-patient, the individual T cell receptor sequences may differ between patients. Despite these differences, different TCR can still recognize the same target. Thus, different T cell receptors may have slight sequence variations from these T cell receptors that can bind the same target. Additionally, T cell receptors may be modified to introduce amino acid substitutions that will allow binding to the same antigen. Such cases include affinity maturation of the T cell receptor for the specific target or receptor modification to improve the specificity of the T cell receptor for its target. The recognition portion of a T cell receptor can be grafted onto other protein scaffolds to be used as a therapeutic reagent. Because T cell receptors are somewhat cross-reactive, the list of synthetic peptides is not exhaustive. Slight modifications to peptide sequences can still result in T cell stimulation.

In some embodiments the T cells from which TCR sequences for screening are obtained are isolated from tumor sites, and may include without limitation tumor infiltrating T cells (TILs). In other embodiments the T cells are obtained from an individual responsive to an infection, e.g. bacterial, viral, protozoan, etc. infection. In other embodiments the T cells are obtained from a graft recipient, and may be isolated from the site of a graft.

### BRIEF DESCRIPTION OF THE DRAWINGS

The invention is best understood from the following detailed description when read in conjunction with the accompanying drawings. The patent or application file contains at least one drawing executed in color. It is emphasized that, according to common practice, the various features of the drawings are not to-scale. On the contrary, the dimensions of the various features are arbitrarily expanded or reduced for clarity. Included in the drawings are the following figures.

FIG. 1. Design of the peptide-HLA-A*02:01 yeast-display library. (A) Methodology for selecting a yeast-display library of pHLA. Each yeast display a unique peptide that is genetically encoded. A typical library contains ˜10⁸ unique peptides, which is selected by a TCR of interest. Yeast are enriched in an affinity-based selection using bead-multimerized TCR and grown for iterative rounds of selection. Peptides are successively enriched and all yeast DNA is deep-sequenced. These synthetic peptide sequences are used to generate a model to make predictions for TCR ligands derived from the human proteome and/or patient-specific exome. (B) The goal of the study is to use the yeast-display selection to de-orphanize a TCR of unknown antigen specificity. The peptides selected by a TCR from the yeast-display selection generates a recognition landscape for a particular TCR, which is then used to make predictions of antigen specificity for orphan TCRs. Predicted targets can be validated in a T cell stimulation assay. (C) The construct utilizes a single-chain design to display the pHLA-A*02:01 complex tethered to an epitope tag and Aga2p, which binds to the native Aga1 protein on yeast. Each component is connected covalently by a Gly-Ser linker. The epitope tag is introduced to monitor expression of the library. (D) The MART-1/HLA-A*02 complex structure (PDB 4L3E) highlighting the two peptide anchors with orange arrows. These peptide positions at P2 and PΩ of the peptide allow for peptide binding to HLA-A*02. (E) An example 8 mer peptide library shows the anchor preferences for the HLA-A*02:01 library and the remaining positions that are randomized to any of the twenty amino acids (X=twenty amino acids and stop codon). Nucleotide abbreviations for codon usage are listed according to the IUPAC nucleotide code. (F) A multi-length library designed to capture the most common length peptides presented by HLA-A*02:01. Each peptide length is placed in a construct using a unique epitope tag for selection monitoring. The libraries have theoretical nucleotide diversities dictated by the peptide length and library composition. The functional diversity represents the true capacity of the physical libraries based on yeast colony counting after limiting dilution of the library.

FIG. 2. Validation of the HLA-A*02:01 library with the DMF5 TCR. (A) The DMF5 TCR stains yeast displaying the MART-1 peptide (ELAGIGILTV) (SEQ ID NO: 264) in complex with HLA-A*02:01 on the surface of yeast. Streptavidin-647 (SA-647) was used to tetramerize and fluorescently label the DMF5 TCR. (B) Enrichment of the 10 mer length HLA-A*02:01 yeast-display library by the DMF5 TCR as measured by anti-HA epitope tag staining by flow cytometry. Three of four rounds of selection shown. (C) Highly-enriched peptides sequenced from the 10 mer selection by the DMF5 TCR are stained by the DMF5 TCR tetramer and measured by flow cytometry. ((C) sequences from left to right: SEQ ID NOs: 264, 324, 286, 323, 283, 285). (D) The fraction of total sequencing read counts of the top 10 peptides according to deep sequencing of round 3 of the 10 mer HLA-A*02:01 library selections by the DMF5 TCR. ((D) sequences from top to bottom: SEQ ID NOs: 287, 326, 325, 324, 286, 323, 285, 322, 284, 283). (E) Unique peptides from round 3 of selection fall into two major clusters that appear similar to the wildtype MART-1 peptide sequence (SEQ ID NO: 267). Clusters are determined by first calculating reverse hamming distance between all peptides present in round 3 of the selection and then clustered by score. The MART-1 decamer structure (PDB: 4L3E) is aligned to the selected peptides. (F) A substitution matrix (2014PWM) using cluster 1 peptides predicts the MART-1 peptide as the most probable peptide to bind the DMF5 TCR among eight other predicted peptides. ((F) sequences from top to bottom: 321, 320, 319, 318, 317, 316, 315, 314, 267)

FIG. 3. Blinded validation of the HLA-A*02:01 library by neoantigen-specific TCRs. (A) Three TCRs of blinded specificity separately enrich the HLA-A*02:01 library for a specific peptide length according to epitope tag staining over the rounds of selection. The left panels indicate tetramer and epitope staining after all 4 rounds of selection have completed and the right panels indicate epitope staining through the course of selections. (B) Unique peptides selected by NKI 2 in round 3 of the selection are parsed by peptide length and clustered by reverse hamming distance. The number of peptides identified in the cluster are shown on the right along with the respective peptide lengths. (C) The maximum reverse hamming distance computed between every 10 mer of the selected peptides by NKI 2 at round 3 and each 10 mer neoantigen peptide from the list of 127 total neoantigens. ((C) sequences from top to bottom: SEQ ID NOs: 501, 502, 620, 503-519. (D) Two peptides Lib-1 (SEQ ID NO: 434) and Lib-2 (SEQ ID NO: 269) from the selected library closely resemble the 10 mer neoantigen peptide ALDPHSGHFV (SEQ ID NO: 265) derived from CDK4. Identical amino acids with the neoantigen are colored in red. (E) The top 5 peptides of length 10 selected by the NKI 2 TCR were used to stimulate peripheral blood lymphocytes transduced to express TCRs NKI1 or NK12, which are both specific for the CDK4 neoantigen ALDPHSGHFV (SEQ ID NO: 265). Transduced lymphocytes were mixed 1:1 with JY cells pulsed with peptide, control peptide, or no peptide, and IFN₁ production as measured by intracellular antibody staining was assessed using flow cytometry. ((E) sequences from top to bottom: 1) SEQ ID NO: 269, 2) SEQ ID NO: 427, 3) SEQ ID NO: 423, 4) SEQ ID NO: 420, 5) SEQ ID NO: 417).

FIG. 4. Profiling TCRs identified in two HLA-A*02 patients with colorectal adenocarcinoma (A) Study design to de-orphanize patient-derived TCRs on the HLA-A*02:01 library with summarized results. (B) Bar graph of abundances of unique paired αβ TCR sequences from TILs.*=TCRs that enriched peptides from the library.(C) Venn diagrams representing the overlap of individual unique CDR3α or CDR3β chain sequences between tumor and healthy tissues for each patient. The number indicates the amount of CDR3 sequences in the nearest section of the Venn diagram. (D) Heatmaps identifying the binary measurement of transcription factors using sequencing of amplified and barcoded transcripts. The alternating black and white panels indicate boundaries of single T cell clones with the same receptor sequences, with the most abundance clones beginning from the left most side. The left panel identifies those T cells with TCRs chosen from Patient A to be screened and green denoting the presence of transcript. The right panel identifies those T cells with TCRs chosen from Patient B to be screened and blue denoting the presence of transcript. White indicates lack of transcript detected. TCRs 1A, 2A, 3B, and 4B are labeled.

FIG. 5. Four TIL-derived TCRs enrich the HLA-A*02:01 library for peptides. (A) TCR sequences of the four orphan TCRs that selected peptides from the HLA-A*02:01 library. The TCR gene segments variable and joining are shown along with the corresponding CDR3 sequence. The abundance represents the amount of times a single cell was found to have the exact TCR sequence in tumor/healthy tissue. ((A)) sequences: 1A CDR3α: (SEQ ID NO: 472), 2A CDR3α: (SEQ ID NO: 261), 3B CDR3α: (SEQ ID NO: 261), 4B CDR3α: (SEQ ID NO: 495), 1A CDR3β: (SEQ ID NO: 463), 2A CDR3β: (SEQ ID NO: 262), 3B CDR3β: (SEQ ID NO: 263), 4B CDR3β: (SEQ ID NO: 484)) (B) Nucleotide sequences of the two sequence-similar TCRs isolated from patients A and B. Non-encoded nucleotides are highlighted in red. ((B) amino acid sequences: CDR3α 2A: (SEQ ID NO: 261), CDR3α 3B: (SEQ ID NO: 261), CDR3β 2A: (SEQ ID NO: 262), CDR3β 3B: (SEQ ID NO: 263)); nucleotide sequences: CDR3α 2A nucleotide sequence: (SEQ ID NO: 536), CDR3α 3B nucleotide sequence: (SEQ ID NO: 537), CDR3β 2A nucleotide sequence: (SEQ ID NO: 538), CDR3β 3B nucleotide sequence (SEQ ID NO: 539). (C) HLA enrichment and tetramer staining per round of selection by the four orphan TCRs as measured by flow cytometry. The left panels indicate tetramer and epitope staining after all 4 rounds of selection have completed and the right panels indicate epitope staining through the course of selections.

FIG. 6. Deep-sequencing results of the yeast selections by the four TIL TCRs. (A) Word logos display the unique round 3 selected peptides for each TCR not accounting for deep sequencing read count abundance. The size of the amino acid letter represents its proportional abundance at the given position among the unique peptides. (B) Heatmap plots showing the amino acid composition per position of the peptide accounting for peptide enrichment at round 3 of the selection. Darker colors indicate greater abundance of a given amino acid at a given position. Anchor residues are outlined in black. (C) TCRs 2A and 3B select an overlapping set of 11 peptides in round 3 of the selection shown as a fraction of total reads in round 3. ((C) sequences from top to bottom: SEQ ID NOs: 95, 249, 54, 195, 42, 191, 196, 198, 200, 201, 4).

FIG. 7. Activation of TIL TCRs with predicted human targets and peptide mimotopes. TCRs are retrovirally infected into CD8⁺ SKW-3 cells and sorted for stable TCR (IP26) and CD3 (UCHT1) co-expression. T2 antigen-presenting cells are pulsed with 100 μM peptide for 3 hours, co-incubated with the T cell lines for 18 hours and analyzed for CD69 expression by flow cytometry. (A) TCR1A, (C) TCR2A, (E) TCR3B, and (G) TCR4B are tested for CD69 activation by peptide stimulation in technical triplicate with standard deviation shown. A representative experiment is shown from biological triplicate. ((A) sequences from left to right: SEQ ID NOs: 540-555; (C) SEQ ID NOs: 556-574; (E) SEQ ID NOs: 556-574; (G) SEQ ID NOs: 596-619). (B), (D), (F), (H) A dose-response curve for each stimulatory peptide is shown on the right plotted with means of biological triplicates with standard error of the mean. For both experiments, p-values are calculated using ordinary one-way ANOVA. For TCRs 2A and 3B, 17 non-stimulating peptides are removed for simplicity. ((B) sequences from top to bottom: SEQ ID NOs: 540-543; (D) sequences from top to bottom: 556-558, 560, 562-567; (F) sequences from top to bottom: 41, 42, 193, 194, 195, 257; (H) sequences from top to bottom: 596-602, 604, 608, 610, 613, 615).

FIG. 8. Validation of the HLA-A2*01 library with the DMF5 TCR. (A) MA2.1 antibody staining for correctly folded HLA-A*02:01 complex with DMF5 TCR wildtype peptide or peptide mimotopes. Histograms show staining by MA2.1 antibody followed by secondary antibody. ((A) sequences from left to right: SEQ ID NOs: 264, 324, 286, 323, 283, 285). (B) The scores of predicted human peptides using the 2014PWM algorithm on cluster 2 of the round 3 sequences for the DMF5 TCR 10 mer selection. (C) The scores of the top 10 peptides identified in FIG. 8B. ((C) sequences from top to bottom: SEQ ID NO: 364, 363, 362, 361, 360, 359, 358, 357, 356, 355).

FIG. 9. Patient tissue immunohistochemistry and TCR repertoire sequencing and phenotyping. (A) Patient immunohistochemistry using H&E staining, anti-CD4/hematoxylin or anti-CD8/hematoxylin. All representative images are taken using 300× magnification. (B) Patient CDR3 length as measured from the Cys to Phe. (C) Patient distribution of TCR variable a genes in healthy and tumor tissue. (D) Patient distribution of TCR variable β genes in healthy and tumor tissue. (E) t-SNE plots of Patient B T cells showing transcriptional profiling by transcript sequencing (left) and cell surface markers by flow cytometry (right). The presence of transcripts is binary based off of deep-sequencing reads (1=yes, 0=no) and intensity relates to MFI of cell surface marker.

FIG. 10. Design of the Machine-Learning Algorithm 2017DL to Predict Human Peptide Specificities. (A) Schematic showing the process to take data from the yeast-display library selections to train a machine learning model, which scores peptides derived from proteins from the Uniprot database or patient-specific exomes. The model is generated from yeast-display selection data utilizing the deep-sequencing round counts per peptide and the composition of the peptide. An exponential curve is fit to each peptide to capture the enrichment over the rounds of selection using a fitness function. (B) Fitness function to fit an exponential curve to the deep sequencing round counts for peptides selected by a TCR. (C) Matrix representation of an example peptide, in which each amino acid is represented as a one-hot vector. (D) The architecture of the machine-learning algorithm utilizing a two-layer convolutional neural network. The input consists of peptide sequences represented as a vector of one-hot vectors and the fitness scores of the peptides determined from the fitness function. The output is the fitness score.

FIG. 11. Activation of SKW-3 cells according to CD69 Median MFI and TCR tetramer staining of yeast expressing predicted peptide targets. Data analyzed from FIG. 7, but using mean fluorescence intensity of CD69 expression instead of percent cells positive for CD69 expression for (A), (B), (C), and (D). SKW-3 T cells with TCRs (A) 1A, (B) 2A, (C) 3B, or (D) 4B were co-cultured with peptide-pulsed T2 antigen-presenting cells as in FIG. 7. The mean fluorescence intensity was measured from anti-CD69 staining of CD3-gated SKW-3 cells. in technical triplicate with mean values and standard deviation shown. A representative experiment from biological triplicate is shown. P-values were measured using ordinary one-way ANOVA. Yeast expressing single-chain trimers of the library peptides and predicted target peptides for TCRs (E) 1A, (F) 2A, (G), 3B, and (H) 4B stained with 400 nM TCR tetramers. Tetramer negative populations are stained with streptavidin-647 only. All yeast are gated on epitope tag positive yeast. ((A) sequences from top to bottom: SEQ ID NOs: 540-542).

FIG. 12. U2AF2 quantitative RNA expression and affinity measurements for U2AF2 peptide. (A) Quantitative PCR expression of the U2AF2 transcript expression of tumor over healthy tissue in patients A and B using 18S as the housekeeping gene. Samples are done in technical quadruplicate with standard deviation shown. (B) Log base 2 quantitative PCR expression of U2AF2 RNA in various human-derived tumors compared to U2AF2 RNA expression in Patient A healthy tissue using the 18S as the housekeeping gene. Samples are done in technical quadruplicate with standard deviation shown. Cell lines shown are listed in the methods section in the appropriate order. (C) Log base 2 quantitative PCR expression of U2AF2 RNA in various human-derived tumors compared to U2AF2 RNA expression in Patient B healthy tissue using the 18S as the housekeeping gene. Samples are done in technical quadruplicate with standard deviation shown. Cell lines shown are listed in the methods section in the appropriate order. (D) Surface plasmon resonance traces of increasing concentrations of TCR 2A flown over a chip coated with MMDFFNAQM-HLA-A*02:01 (SEQ ID NO: 266) with a range of 93.6 μM to 0.365 μM using 2-fold dilutions. The peaks prior to and after association of the TCR to the peptide-HLA-A*02 generated from flow cell subtraction are removed for simplicity. Only the colored curves labeled with concentrations are used to calculate the K_{d}. (E) Curve-fitting to data points generated at various concentrations of TCR labeled in Figure S4D.

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Screening methods. Antigenic sequences were discovered by generating a library of single chain polypeptides that comprise: the binding domains of a major histocompatibility complex protein; and diverse peptide ligands. The library was introduced into a suitable host cell that expresses the encoded polypeptide, which host cells include, without limitation, yeast cells. A TCR of interest is multimerized to enhance binding, and used to select for host cells expressing those single chain polypeptides that bind to the T cell receptor. Iterative rounds of selection are performed, i.e. the cells that are selected in the first round provide the starting population for the second round, etc. until the selected population has a signal above background, usually at least three and more usually at least four rounds of selection are performed. Polynucleotides encoding the final selected population from the library of single chain polypeptides are subjected to high throughput sequencing. The selected set of peptide ligands exhibit a restricted choice of amino acids at residues, e.g. the residues that contact the TCR, which information can be input into an algorithm that can be used to analyze public databases for all peptides that meet the criteria for binding, and which provides a set of peptides that meet these criteria.

The peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids. It will be appreciated that a fully random library would represent an extraordinary number of possible combinations. In preferred methods, the diversity is limited at the residues that anchor the peptide to the MHC binding domains, which are referred to herein as MHC anchor residues. The position of the anchor residues in the peptide are determined by the specific MHC binding domains. Class I binding domains can have anchor residues at the P2 position, and at the last contact residue. Class II binding domains have an anchor residue at P1, and depending on the allele, at one of P4, P6 or P9. For example, the anchor residues for IE^{k} are P1 {I,L,V} and P9 {K}; the anchor residues for HLA-DR15 are P1 {I,L,V} and P4 {F, Y}. Anchor residues for DR alleles are shared at P1, with allele-specific anchor residues at P4, P6, P7, and/or P9.

In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts. In certain specific embodiments, the binding domains are HLA-DR4α comprising the set of amino acid changes {M36L, V132M}; and HLA-DR4β comprising the set of amino acid changes {H62N, D72E}. In certain specific embodiments, the binding domains are HLA-DR15αcomprising the set of amino acid changes {F12S, M23K}; and HLA-DR15β comprising the amino acid change {P11S}. In certain specific embodiments, the binding domains are H2 IE^{k}α comprising the set of amino acid changes {I8T, F12S, L14T, A56V} and H2 IE^{k}β comprising the set of amino acid changes {W6S, L8T, L34S}.

In some embodiments, the binding domains of a major histocompatibility complex protein comprise the alpha 1 and alpha 2 domains of a Class I MHC protein, which are provided in a single chain with β2 microglobulin. In some such embodiments the Class I protein has been subjected to mutagenesis and selected for amino acid changes that enhance the solubility of the single chain polypeptide, without altering the peptide binding contacts. In certain specific embodiments, the binding domains are HLA-A2 alpha 1 and alpha 2 domains, comprising the amino acid change {Y84A}. In certain specific embodiments, the binding domains are H2-L^{d} alpha 1 and alpha 2 domains, comprising the amino acid change {M31R}. In certain specific embodiments the binding domains are HLA-B57 alpha 1, alpha 2 and alpha 3 domains, comprising the amino acid change {Y84A}.

The sequences of peptides are determined by any convenient methods of high throughput sequencing. Sequences may be analyzed, for example by the methods disclosed in the Examples, using clustering algorithms. Peptides may be analyzed to search human protein (Uniprot) or patient-specific exomes to score peptides of fixed lengths using a sliding window. Substitution matrices are made by determining the frequency of all amino acids per position of the peptide. A cutoff of 0.1% frequency for an amino acid at a given position may be instituted to remove noise.

To determine the statistical significance of a peptide, the human proteome and exome peptide set is scored. To calculate the p-values for the exome peptide set, the percentile score is calculated in context of the human proteome scores. The uncorrected p-value is 1-percentile. The Bonferroni-corrected p-value is the uncorrected p-value multiplied by the number of peptides in the mutant set.

MHC Proteins. Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class II.

The MHC proteins used in the libraries and methods of the invention may be from any mammalian or avian species, e.g. primate sp., particularly humans; rodents, including mice, rats and hamsters; rabbits; equines, bovines, canines, felines; etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β₂-microglobulin. Included in the murine H-2 subunits are the class I H-2K, H-2D, H-2L, and the class II I-Aα, I-Aβ, I-Eα and I-Eβ, and β₂-microglobulin.

The MHC binding domains are typically a soluble form of the normally membrane-bound protein. The soluble form is derived from the native form by deletion of the transmembrane domain. Conveniently, the protein is truncated, removing both the cytoplasmic and transmembrane domains. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ and DR) exists in different alleles. Current nomenclature for HLA alleles are designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), The IMGT/HLA database. Nucleic Acids Research 39 Suppl 1:D1171-6, herein specifically incorporated by reference.

The numbering of amino acid residues on the various MHC proteins and variants disclosed herein is made to be consistent with the full length polypeptide. Boundaries were set to either be the end of the MHC peptide binding domain (as judged by examining crystal structures) for the ‘mini’ MHCs, e.g. as exemplified herein with I-Ek, H2-Ld, and HLA-DR15, and the end of the Beta2/Alpha2/Alpha3 domains as judged by structure and/or sequence for the ‘full length’ MHCs, as exemplified herein with HLA-A2, -B57, and -DR4.

In some embodiments, the MHC portion of a construct is the MHC portion delineated in any of SEQ ID NO:1-6. It will be understood by one of skill in the art that the peptide and linker portions can be varied from the provided sequences.

MHC context. The function of MHC molecules is to bind peptide fragments derived from pathogens and display them on the cell surface for recognition by the appropriate T cells. Thus T cell receptor recognition can be influenced by the MHC protein that is presenting the antigen. The term MHC context refers to the recognition by a TCR of a given peptide, when it is presented by a specific MHC protein.

Class II HLA/MHC. Class II binding domains generally comprise the α1 and α2 domains for the a chain, and the β1 and β2 domains for the β chain. Not more than about 10, usually not more than about 5, preferably none of the amino acids of the transmembrane domain will be included. The deletion will be such that it does not interfere with the ability of the α2 or β2 domain to bind peptide ligands.

In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

In certain specific embodiments, the binding domains are an HLA-DR allele. The HLA-DRA protein can be selected, without limitation, from the binding domains of DRA*01:01:01:01; DRA*01:01:01:02; DRA*01:01:01:03; DRA*01:01:02; DRA*01:02:01; DRA*01:02:02; and DRA*01:02:03, which may be modified to comprise the amino acid changes {M36L, V132M}; or {F125, M23K}, depending on whether it is provided in the context of a full-length or mini-allele. The HLA-DRA binding domains can be combined with any one of the HLA-DRB binding domains.

In certain such embodiments, the HLA-DRA allele is paired with the binding domains of an HLA-DRB4 allele. The HLA-DRB4 allele can be selected from the publicly available DRB4 alleles.

In other such embodiments the HLA-DRA allele is paired with the binding domains of an HLA-DRB15 allele. The HLA-DRB15 allele can be selected from the publicly available DRB15 alleles.

In other embodiments the Class II binding domains are an H2 protein, e.g. I-Aα, I-Aβ, I-Eα and I-Eβ. In some such embodiments, the binding domains are H2 IE^{k}α which may comprise the set of amino acid changes {I8T, F12S, L14T, A56V}; and H2 IE^{k}β which may comprise the set of amino acid changes {W6S, L8T, L34S}.

Class I HLA/MHC. For class I proteins, the binding domains may include the α1, α2 and α3 domain of a Class I allele, including without limitation HLA-A, HLA-B, HLA-C, H-2K, H-2D, H-2L, which are combined with β₂-microglobulin. Not more than about 10, usually not more than about 5, preferably none of the amino acids of the transmembrane domain will be included. The deletion will be such that it does not interfere with the ability of the domains to bind peptide ligands.

In certain specific embodiments, the binding domains are HLA-A2 binding domains, e.g. comprising at least the alpha 1 and alpha 2 domains of an A2 protein. A large number of alleles have been identified in HLA-A2, including without limitation HLA-A*02:01:01:01 to HLA-A*02:478, which sequences are available at, for example, Robinson et al. (2011), The IMGT/HLA database. Nucleic Acids Research 39 Suppl 1:D1171-6. Among the HLA-A2 allelic variants, HLA-A*02:01 is the most prevalent. The binding domains may comprise the amino acid change {Y84A}.

In certain specific embodiments, the binding domains are HLA-B57 binding domains, e.g. comprising at least the alpha1 and alpha 2 domains of a B57 protein. The HLA-B57 allele can be selected from the publicly available B57 alleles.

T cell receptor, refers to the antigen/MHC binding heterodimeric protein product of a vertebrate, e.g. mammalian, TCR gene complex, including the human TCR α, β, γ and δ chains. For example, the complete sequence of the human β TCR locus has been sequenced, as published by Rowen et al. (1996) Science 272(5269):1755-1762; the human a TCR locus has been sequenced and resequenced, for example see Mackelprang et al. (2006) Hum Genet. 119(3):255-66; see a general analysis of the T-cell receptor variable gene segment families in Arden Immunogenetics. 1995;42(6):455-500; each of which is herein specifically incorporated by reference for the sequence information provided and referenced in the publication.

The multimerized T cell receptor for selection in the methods of the invention is a soluble protein comprising the binding domains of a TCR of interest, e.g. TCRα/β, TCRγ/δ. The soluble protein may be a single chain, or more usually a heterodimer. In some embodiments, the soluble TCR is modified by the addition of a biotin acceptor peptide sequence at the C terminus of one polypeptide. After biotinylation at the acceptor peptide, the TCR can be multimerized by binding to biotin binding partner, e.g. avidin, streptavidin, traptavidin, neutravidin, etc. The biotin binding partner can comprise a detectable label, e.g. a fluorophore, mass label, etc., or can be bound to a particle, e.g. a paramagnetic particle. Selection of ligands bound to the TCR can be performed by flow cytometry, magnetic selection, and the like as known in the art.

Peptide ligands of the TCR are peptide antigens against which an immune response involving T lymphocyte antigen specific response can be generated. Such antigens include antigens associated with autoimmune disease, infection, foodstuffs such as gluten, etc., allergy or tissue transplant rejection. Antigens also include various microbial antigens, e.g. as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and self-antigens, e.g. her-2/neu; carcinoembryonic antigen, muc-1, and the like.

In the methods of the invention, a library of diverse peptide antigens is generated.

The peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids. It will be appreciated that a fully random library would represent an extraordinary number of possible combinations. In preferred methods, the diversity is limited at the residues that anchor the peptide to the MHC binding domains, which are referred to herein as MHC anchor residues. The position of the anchor residues in the peptide are determined by the specific MHC binding domains. Diversity may also be limited at other positions as informed by binding studies, e.g. at TCR anchors.

Library. In some embodiments of the invention, a library is provided of polypeptides, or of nucleic acids encoding such polypeptides, wherein the polypeptide structure has the formula:

polynucleotide composition encoding the P-L₁-β-L₂-α-L₃-T polypeptide
wherein each of L₁, L₂ and L₃ are flexible linkers of from about 4 to about 12 amino acids in length, e.g. comprising glycine, serine, alanine, etc.
α is a soluble form of a domains of a class I MHC protein, or class II α MHC protein;
β is a soluble form of (i) a β chain of a class II MHC protein or (ii) β₂ microglobulin for a class I MHC protein;
T is a domain that allows the polypeptide to be tethered to a cell surface, including without limitation yeast Aga2; and
P is a peptide ligand, usually a library of different peptide ligands as described above, where at least 10⁶, at least 10⁷, more usually at least 10⁸ different peptide ligands are present in the library.

Conventional methods of assembling the coding sequences can be used. In order to generate the diversity of peptide ligands, randomization, error prone PCR, mutagenic primers, and the like as known in the art are used to create a set of polynucleotides. The library of polynucleotides is typically ligated to a vector suitable for the host cell of interest. In various embodiments the library is provided as a purified polynucleotide composition encoding the P-L₁-β-L₂-α-L₃-T polypeptides; as a purified polynucleotide composition encoding the P-L₁-β-L₂-α-L₃-T polypeptides operably linked to an expression vector, where the vector can be, without limitation, suitable for expression in yeast cells; as a population of cells comprising the library of polynucleotides encoding the P-L₁-β-L₂-α-L₃-T polypeptides, where the population of cells can be, without limitation yeast cells, and where the yeast cells may be induced to express the polypeptide library.

“Suitable conditions” shall have a meaning dependent on the context in which this term is used. That is, when used in connection with binding of a T cell receptor to a polypeptide of the formula polynucleotide composition encoding the P-L₁-β-L₂-α-L₃-T polypeptide, the term shall mean conditions that permit a TCR to bind to a cognate peptide ligand. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

The term “specificity” refers to the proportion of negative test results that are true negative test result. Negative test results include false positives and true negative test results.

The term “sensitivity” is meant to refer to the ability of an analytical method to detect small amounts of analyte. Thus, as used here, a more sensitive method for the detection of amplified DNA, for example, would be better able to detect small amounts of such DNA than would a less sensitive method. “Sensitivity” refers to the proportion of expected results that have a positive test result.

The term “reproducibility” as used herein refers to the general ability of an analytical procedure to give the same result when carried out repeatedly on aliquots of the same sample.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167,046, and U.S. Pat. Nos. 7501245; 7491498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Expression construct: Sequences encoding a peptide disclosed herein or a TCR disclosed herein may be introduced on an expression vector, e.g. into a cell to be engineered, as a vaccine, etc. The TCR sequence may be introduced at the site of the endogenous gene, e.g., using CRISPR technology (see, for example Eyquem et al. (2017) Nature 543:113-117; Ren et al. (2017) Protein & Cell 1-10; Ren et al. (2017) Oncotarget 8(10):17002-17011).

Amino acid sequence variants are prepared by introducing appropriate nucleotide changes into the coding sequence, as described herein. Such variants represent insertions, substitutions, and/or specified deletions of, residues as noted. Any combination of insertion, substitution, and/or specified deletion is made to arrive at the final construct, provided that the final construct possesses the desired biological activity as defined herein.

The nucleic acid encoding the sequence is inserted into a vector for expression and/or integration. Many such vectors are available. For example, the CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

The sequences may be produced recombinantly as a fusion polypeptide with a heterologous polypeptide, e.g., a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to the coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryotic, yeast, or other eukaryotic cells described above. Examples of useful mammalian host cell lines are mouse L cells (L-M[TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including engineered T cells, etc. can be transfected with the above-described expression vectors. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

In the event the polypeptides or nucleic acids of the disclosure are “substantially pure,” they can be at least about 60% by weight (dry weight) the biomolecule of interest. For example, the composition can be at least about 75%, about 80%, about 85%, about 90%,about 95% or about 99%, by weight, the biomolecule of interest. Purity can be measured by any appropriate standard method, for example, column chromatography, polyacrylamide gel electrophoresis, or HPLC analysis.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition can be a vector suitable for introducing the sequence into a targeted cell for expression. The label on or associated with the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

The terms “polypeptide,” “protein” or “peptide” refer to any chain of amino acid residues, regardless of its length or post-translational modification (e.g., glycosylation or phosphorylation).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

The peptides disclosed herein can be flanked with additional amino acid residues so long as the peptide retains its TCR inducibility. Such peptides can be less than about 40 amino acids, for example, less than about 20 amino acids, for example, less than about 15 amino acids. The amino acid sequence flanking the peptides consisting of the amino acid sequence selected from the group of SEQ ID NOs: 3-5, 7-9, 12, 15-19, 22, 24, 27-30, 37, 67 and 74 is not limited and can be composed of any kind of amino acids so long as it does not inhibit the TCR recognition. The amino acid sequence may be modified by substituting wherein one or more amino acids. One of skill in the art will recognize that individual additions or substitutions to an amino acid sequence which alters a single amino acid or a small percentage of amino acids results in the conservation of the properties of the original amino acid side-chain; it is thus is referred to as “conservative substitution” or “conservative modification”, wherein the alteration of a protein results in a protein with similar functions.

In addition to the above-mentioned sequence modification of the peptides, the peptides can be further linked to other substances, so long as they retain the TCR binding activity. Usable substances include: peptides, lipids, sugar and sugar chains, acetyl groups, natural and synthetic polymers, etc. The peptides can contain modifications such as glycosylation, side chain oxidation, or phosphorylation; so long as the modifications do not destroy the biological activity of the peptides as described herein. These kinds of modifications can be performed to confer additional functions (e.g., targeting function, and delivery function) or to stabilize the polypeptide.

For example, to increase the in vivo stability of a polypeptide, it is known in the art to introduce particularly useful various D-amino acids, amino acid mimetics or unnatural amino acids; this concept can also be adopted for the present polypeptides. The stability of a polypeptide can be assayed in a number of ways. For instance, peptidases and various biological media, such as human plasma and serum, have been used to test stability (see, e.g., Verhoef et al., Eur J Drug Metab Pharmacokin 11: 291-302, 1986). [0053] III. Preparation of the peptides

The peptides disclosed herein can be prepared using well known techniques. For example, the peptides can be prepared synthetically, by recombinant DNA technology or chemical synthesis. Peptides disclosed herein can be synthesized individually or as longer polypeptides comprising two or more peptides (e.g., two or more peptides or a peptide and a non-peptide). The peptides can be isolated i.e., purified to be substantially free of other naturally occurring host cell proteins and fragments thereof, e.g., at least about 70%, 80% or 90% purified.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, a “therapeutically effective amount” refers to that amount of the therapeutic agent, e.g. an infusion of primed T cells, a peptide or polynucleotide vaccine, etc, sufficient to treat or manage a disease or disorder. A therapeutically effective amount may refer to the amount of therapeutic agent sufficient to delay or minimize the onset of disease, e.g., to delay or minimize the spread of cancer, or the amount effective to decrease or increase signaling from a receptor of interest. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

As used herein, the terms “cancer” (or “cancerous”), or “tumor” are used to refer to cells having the capacity for autonomous growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. Pathologic hyperproliferative cells occur in disease states characterized by malignant tumor growth. Examples of non-pathologic hyperproliferative cells include proliferation of cells associated with wound repair. The terms “cancer” or “tumor” are also used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art-recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Exemplary cancer types include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectal cancer, endometrial cancer, esophagus cancer, Ewing's family of tumors (e.g., Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

### Methods and Compositions

Compositions and methods are provided for accurately identifying the set of peptides recognized by a T cell receptor in a given MHC context; and provide antigens obtained from such screening using a multiplex method to simultaneously screen 2, 3, 4, 5, or more libraries. The peptide ligand (antigen) thus identified is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids, and may include any of the peptides provided herein as SEQ ID NO:1-257.

Selection for a peptide that binds to the TCR of interest is performed by combining a multimerized TCR with the population of host cells expressing the library. The multimerized T cell receptor for selection is a soluble protein comprising the binding domains of a TCR of interest, e.g. a/I3, TCR-y/6, and can be synthesized by any convenient method. The TCR may be a single chain, or a heterodimer. In some embodiments, the soluble TCR is modified by the addition of a biotin acceptor peptide sequence at the C terminus of one polypeptide. After biotinylation at the acceptor peptide, the TCR can be multimerized by binding to biotin binding partner, e.g. avidin, streptavidin, traptavidin, neutravidin, etc. The biotin binding partner can comprise a detectable label, e.g. a fluorophore, mass label, etc., or can be bound to a particle, e.g. a paramagnetic particle. Selection of ligands bound to the TCR can be performed by flow cytometry, magnetic selection, and the like as known in the art.

Rounds of selection are performed until the selected population has a signal above background, usually at least three and more usually at least four rounds of selection are performed. In some embodiments, initial rounds of selection, e.g. until there is a signal above background, are performed with a TCR coupled to a magnetic reagent, such as a superparamagnetic microparticle, which may be referred to as “magnetized”. Herein incorporated by reference, Molday (U.S. Pat. No. 4,452,773) describes the preparation of magnetic iron-dextran microparticles and provides a summary describing the various means of preparing particles suitable for attachment to biological materials. A description of polymeric coatings for magnetic particles used in high gradient magnetic separation (HGMS) methods are found in U.S. Pat. No. 5,385,707. Methods to prepare superparamagnetic particles are described in U.S. Pat. No. 4,770,183. The microparticles will usually be less than about 100 nm in diameter, and usually will be greater than about 10 nm in diameter. The exact method for coupling is not critical to the practice of the invention, and a number of alternatives are known in the art. Direct coupling attaches the TCR to the particles. Indirect coupling can be accomplished by several methods. The TCR may be coupled to one member of a high affinity binding system, e.g. biotin, and the particles attached to the other member, e.g. avidin. Alternatively one may also use second stage antibodies that recognize species-specific epitopes of the TCR, e.g. anti-mouse Ig, anti-rat Ig, etc. Indirect coupling methods allow the use of a single magnetically coupled entity, e.g. antibody, avidin, etc., with a variety of separation antibodies.

Alternatively, and in a preferred embodiment for final rounds of selection, the TCR is multimerized to a reagent having a detectable label, e.g. for flow cytometry, mass cytometry, etc. For example, FACS sorting can be used to increase the concentration of the cells of having a peptide ligand binding to the TCR. Techniques include fluorescence activated cell sorters, which can have varying degrees of sophistication, such as multiple color channels, low angle and obtuse light scattering detecting channels, impedance channels, etc.

After a final round of selection, polynucleotides are isolated from the selected host cells, and the sequence of the selected peptide ligands are determined, usually by high throughput sequencing. It is shown herein that the selection process results in determination of a set of peptides that are bound by the TCR in the specific HLA context. The biological activity of these ligands in the activation of T cells has been validated. The set of selected ligands provides information about the restrictions on amino acid positions required for binding to the T cell receptor. Usually a plurality of peptide ligands are selected, e.g. up to 10, up to 100, up to 500, up to 1000 or more different peptide sequences.

The sequence data from this selected set of peptide ligands provides information about the restrictions on amino acids at each position of the peptide ligand. This can be shown graphically. The restrictions can be particularly relevant at the residues contacting the TCR. Data regarding the restrictions on amino acids at positions of the peptide are input to design a search algorithm for analysis of public databases. The results of the search provide a set of peptides that meet the criteria for binding to the TCR in the MHC context. The search algorithm is usually embodied as a program of instructions executable by computer and performed by means of software components loaded into the computer.

The peptides and T cell receptors that are identified by these methods may be used in vaccine methods, screening methods to classify patient T cell populations, to prime T cells in vitro, and the like.

In some embodiments, the compositions comprise one or more peptides that elicit an immune response to cancer cells, e.g. colorectal cancer cells, in a subject with at least one HLA allele that is HLA-A2. In another aspect, the invention provides compositions comprising a polynucleotide encoding a peptide disclosed herein. In some embodiments, the compositions comprise a plurality (i.e., two or more) polynucleotides encoding a plurality of peptides disclosed herein. In some embodiments, the compositions comprise a polynucleotide that encodes a plurality of peptides disclosed herein.

In a related aspect, methods are provided for treating cancer (e.g., reducing tumor cell growth, promoting tumor cell death) by administering to an individual a peptide or a polynucleotide encoding a peptide disclosed herein. In a related aspect, isolated primed T cells that have been primed with a peptide disclosed herein are provided. In another aspect, an antigen-presenting cell is provided, which comprises a complex formed between an HLA antigen and a peptide disclosed herein. In some embodiments, the antigen presenting cell is isolated.

The term “vaccine” (also referred to as an immunogenic composition) refers to a substance that has the function to induce anti-tumor (or anto-pathogen) immunity upon inoculation into animals.

Cancers to be treated by the pharmaceutical agents are not limited and include all kinds of cancers wherein the corresponding protein to a peptide identified herein is expressed in the subject. Exemplified cancers carcinomas, e.g. colorectal carcinomas.

If needed, the pharmaceutical agents, composed of either a peptide or a polynucleotide encoding a peptide, can optionally include other therapeutic substances as an active ingredient, so long as the substance does not inhibit the TCR stimulating effect of the peptide of interest. For example, formulations can include anti-inflammatory agents, pain killers, chemotherapeutics, and the like. In addition to including other therapeutic substances in the medicament itself, the medicaments can also be administered sequentially or concurrently with the one or more other pharmacologic agents. The amounts of medicament and pharmacologic agent depend, for example, on what type of pharmacologic agent(s) is/are used, the disease being treated, and the scheduling and routes of administration.

The peptides can be administered directly as a pharmaceutical agent, if necessary, that has been formulated by conventional formulation methods. In such cases, in addition to the peptides, carriers, excipients, and such that are ordinarily used for drugs can be included as appropriate without particular limitations. Examples of such carriers are sterilized water, physiological saline, phosphate buffer, culture fluid and such. Furthermore, the pharmaceutical agents can contain as necessary, stabilizers, suspensions, preservatives, surfactants and such. The pharmaceutical agents can be used for treating and/or preventing cancer.

The peptides can be prepared in a combination, which comprises two or more of peptides disclosed herein, to stimulate T cells in vivo. The peptides can be in a cocktail or can be conjugated to each other using standard techniques. For example, the peptides can be expressed as a single polypeptide sequence. The peptides in the combination can be the same or different. By administering the peptides, the peptides are presented at a high density on the HLA antigens of antigen-presenting cells, then T cells that specifically react toward the complex formed between the displayed peptide and the HLA antigen are stimulated. Alternatively, antigen presenting cells that have immobilized the peptides on their cell surface are obtained by removing dendritic cells from the subjects, which are stimulated by the peptides, then endogenous T cells are stimulated in the subjects by readministering the peptide-loaded dendritic cells to the subjects, and as a result, aggressiveness towards the target cells can be increased.

The pharmaceutical agents comprising a peptide described herein as the active ingredient, optionally can comprise an adjuvant so that cellular immunity will be established effectively, or they can be administered with other active ingredients, and they can be administered by formulation into granules. An adjuvant refers to a compound that enhances the immune response against the protein when administered together (or successively) with the protein having immunological activity. An adjuvant that can be applied includes those described in the literature. Exemplary adjuvants include aluminum phosphate, aluminum hydroxide, alum, cholera toxin, salmonella toxin, and such, but are not limited thereto.

Furthermore, liposome formulations, granular formulations in which the peptide is bound to few-mcm diameter beads, and formulations in which a lipid is bound to the peptide can be conveniently used. Alternatively, intracellular vesicles called exosomes are provided, which present complexes formed between the peptides and HLA antigens on their surface. The exosomes can be inoculated as vaccines, similarly to the peptides.

In some embodiments the pharmaceutical agents disclosed herein comprise a component that primes T lymphocytes. Lipids have been identified as agents capable of priming CTL in vivo against viral antigens. For example, palmitic acid residues can be attached to the epsilon-and alpha-amino groups of a lysine residue and then linked to a peptide disclosed herein. The lipidated peptide can then be administered either directly in a micelle or particle, incorporated into a liposome, or emulsified in an adjuvant. As another example of lipid priming of CTL responses, E. coli lipoproteins, such as tripalmitoyl-S-glycerylcysteinlyseryl-serine (P3CSS) can be used to prime CTL when covalently attached to an appropriate peptide (see, e.g., Deres et al., Nature 342: 561, 1989).

The method of administration can be oral, intradermal, subcutaneous, intravenous injection, or such, and systemic administration or local administration to the vicinity of the targeted sites finds use. The administration can be performed by single administration or boosted by multiple administrations. The dose of the peptides can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once every a few days to once every few months. One skilled in the art can appropriately select the suitable dose.

The pharmaceutical agents disclosed herein can also comprise nucleic acids encoding the peptides disclosed herein in an expressible form. Herein, the phrase “in an expressible form” means that the polynucleotide, when introduced into a cell, will be expressed in vivo as a polypeptide that has stimulates anti-tumor immunity. In one embodiment, the nucleic acid sequence of the polynucleotide of interest includes regulatory elements necessary for expression of the polynucleotide in a target cell. The polynucleotide(s) can be equipped to stably insert into the genome of the target cell (see, e.g., Thomas K R & Capecchi M R, Cell 51: 503-12, 1987 for a description of homologous recombination cassette vectors). See, e.g., Wolff et al., Science 247: 1465-8, 1990; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

The peptides disclosed herein can also be expressed by viral or bacterial vectors.

Examples of expression vectors include attenuated viral hosts, such as vaccinia or fowlpox. This approach involves the use of vaccinia virus, e.g., as a vector to express nucleotide sequences that encode the peptide. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351: 456-60, 1991. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al., Mol Med Today 6: 66-71, 2000; Shedlock et al. J Leukoc Biol 68: 793-806, 2000; Hipp et al., In Vivo 14: 571-85, 2000.

The method of administration can be oral, intradermal, subcutaneous, intravenous injection, or such, and systemic administration or local administration to the vicinity of the targeted sites finds use. The administration can be performed by single administration or boosted by multiple administrations. The dose of the polynucleotide in the suitable carrier or cells transformed with the polynucleotide encoding the peptides can be adjusted appropriately according to the disease to be treated, age of the patient, weight, method of administration, and such, and is ordinarily 0.001 mg to 1000 mg, for example, 0.001 mg to 100 mg, for example, 0.1 mg to 10 mg, and can be administered once every a few days to once every few months. One skilled in the art can appropriately select the suitable dose.

Also provided are antigen-presenting cells (APCs) that present complexes formed between HLA antigens and the peptides on its surface. APCs are obtained by contacting the peptides, or the nucleotides encoding the peptides, and can be prepared from subjects who are the targets of treatment and/or prevention, and can be administered as vaccines by themselves or in combination with other drugs including the peptides, exosomes, or cytotoxic T cells. The APCs are not limited to any kind of cells and includes dendritic cells (DCs), Langerhans cells, macrophages, B cells, and activated T cells, all of which are known to present proteinaceous antigens on their cell surface so as to be recognized by lymphocytes. Since DC is a representative APC having the strongest CTL inducing action among APCs, DCs find particular use as the APCs.

For example, an APC can be obtained by inducing dendritic cells from the peripheral blood monocytes and then contacting (stimulating) them with the peptides in vitro, ex vivo or in vivo. When the peptides are administered to the subjects, APCs that have the peptides immobilized to them are stimulated in the body of the subject, “inducing APC” includes contacting (stimulating) a cell with the peptides, or nucleotides encoding the peptides to present complexes formed between HLA antigens and the peptides on cell's surface. Alternatively, after immobilizing the peptides to the APCs, the APCs can be administered to the subject as a vaccine. For example, the ex vivo administration can comprise steps of: a: collecting APCs from subject: and b: contacting with the APCs of step a, with the peptide. The APCs obtained by step b can be administered to the subject as a vaccine.

Such APCs can be prepared by a method which comprises the step of transferring genes comprising polynucleotides that encode the peptides to APCs in vitro. The introduced genes can be in the form of DNAs or RNAs. For the method of introduction, without particular limitations, various methods conventionally performed in this field, such as lipofection, electroporation, and calcium phosphate method can be used.

Cells may be engineered to express a TCR provided here, or to respond to a peptide antigen provided herein. A number of different cell types are suitable for engineering, particularly T cells or NK cells. In some embodiments the cells for engineering are autologous. In some embodiments the cells are allogeneic.

A T cell stimulated against any of the peptides disclosed herein can be used as vaccines similar to the peptides. Thus, the present invention provides isolated T cells that are stimulated by any of the present peptides. Such T cells can be obtained by (1) administering to a subject or (2) contacting (stimulating) subject-derived APCs, and CD8-positive cells, or peripheral blood mononuclear leukocytes in vitro with the peptide. T cells, which have been stimulated by stimulation from APCs that present the peptides, can be derived from subjects who are targets of treatment and/or prevention, and can be administered by themselves or in combination with other drugs including the peptides or exosomes for the purpose of regulating effects. The obtained T cells act specifically against target cells presenting the peptides, for example, the same peptides used for priming. The target cells can be cells that express endogenously, or cells that are transfected with genes, and cells that present the peptides on the cell surface due to stimulation by these peptides can also become targets of attack.

In some embodiments, the engineered cell is a T cell. The term “T cells” refers to mammalian immune effector cells that may be characterized by expression of CD3 and/or T cell antigen receptor, which cells can be engineered to express a TCR provided herein or stimulated to respond to a peptide provided herein. In some embodiments the T cells are selected from naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, T stem cell memory cells (T_{SCM}). effector memory T cells, NKT cells, γδ T cells. In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et α1 (2015) Semin Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”. In some embodiments, T cells are contacted with a peptide in vitro, i.e. where the T cells are then transferred to a recipient.

Effector cells, for the purposes of the invention, can include autologous or allogeneic immune cells having cytolytic activity against a target cell, including without limitation tumor cells. The effector cells can be obtained by engineering peripheral blood lymphocytes (PBL) in vitro, then culturing with a cytokine and/or antigen combination that increases activation. The cells are optionally separated from non-desired cells prior to culture, prior to administration, or both. Cell-mediated cytolysis of target cells by immunological effector cells is believed to be mediated by the local directed exocytosis of cytoplasmic granules that penetrate the cell membrane of the bound target cell.

Cytotoxic T lymphocytes (CTL) reactive to tumor cells are specific effector cells for adoptive immunotherapy and are of interest for engineering by priming with peptides disclosed herein, or engineering to express a TCR disclosed herein. Induction and expansion of CTL is antigen-specific and MHC restricted.

T cells collected from a subject may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS).

The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

The peptide and T cell receptor sequences are also useful in screening assays for patient samples, where a T cell containing sample from an individual, e.g. a blood sample, tumor biopsy sample, lymph node sample, bone marrow sample, etc. is analyzed for (i) the presence of T cells comprising a TCR identified herein, and/or (ii) the presence of T cells response to a peptide described herein. The determination of the presence of T cells may be made according to any convenient method, e.g. determining stimulation by measuring proliferation, etc., in response to the presence of the peptide in an HLA complex, or as presented by an APC. The presence of a specific TCR may be determined by sequencing of mRNA, sequencing of genomic DNA, etc. The presence of T cells responsive to the peptide or having a TCR of interest allows the patient to be assigned to a group that can be treated by vaccination, APC transfer, etc. with that group.

Also provided herein are software products tangibly embodied in a machine-readable medium, the software product comprising instructions operable to cause one or more data processing apparatus to perform operations comprising: generating a n×20 matrix from the positional frequencies of selected peptide ligands obtained by the screening methods of the invention, where n is the number of amino acid positions in the peptide ligand library. A cutoff of amino acid frequencies is set, e.g. less than 0.1, less than 0.05, less than 0.01, and frequencies below the cutoff are set to zero. A database of sequences, e.g. a set of human polypeptide sequences; a set of pathogen polypeptide sequences, a set of microbial polypeptide sequences, a set of allergen polypeptide sequences; etc. are searched with the algorithm using an n-position sliding window alignment with scoring the product of positional amino acid frequencies from the substitution matrix. An aligned segment containing at least one amino acid where the frequency is below the cutoff is excluded as a match. The results of the search can be output as a data file in a computer readable medium

The peptide sequence results and database search results may be provided in a variety of media to facilitate their use. “Media” refers to a manufacture that contains the expression repertoire information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

The search algorithm and sequence analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Further provided herein is a method of storing and/or transmitting, via computer, sequence, and other, data collected by the methods disclosed herein. Any computer or computer accessory including, but not limited to software and storage devices, can be utilized to practice the present invention. Sequence or other data can be input into a computer by a user either directly or indirectly. Additionally, any of the devices which can be used to sequence DNA or analyze DNA or analyze peptide binding data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### EXPERIMENTAL

### Example 1

### Antigen Identification for Orphan T Cell Receptors Expressed on Tumor-Infiltrating Lymphocytes

The immune system can mount T cell responses against tumors; however, the antigen specificities of tumor-infiltrating lymphocytes (TILs) are not well understood. Given recent findings that TCRs often exhibit strong preferences for their endogenous ligands, we used yeast-display libraries of peptide-human leukocyte antigen (pHLA) to screen for antigens of ‘orphan’ T cell receptors (TCRs) expressed on TILs from human colorectal adenocarcinoma. Four TIL-derived TCRs exhibited strong selection for peptides presented in a highly diverse pHLA-A*02:01 library. Three of the TIL TCRs were specific for non-mutated self-antigens, two of which were present in separate patient tumors, and shared specificity for a non-mutated self-antigen derived from U2AF2. These results show that the limited recognition surface of MHC-bound peptide accessible to the TCR contains sufficient structural information to enable reconstruction of sequences of peptide targets for pathogenic TCRs of unknown specificity. This finding has enabled the facile identification of tumor antigens.

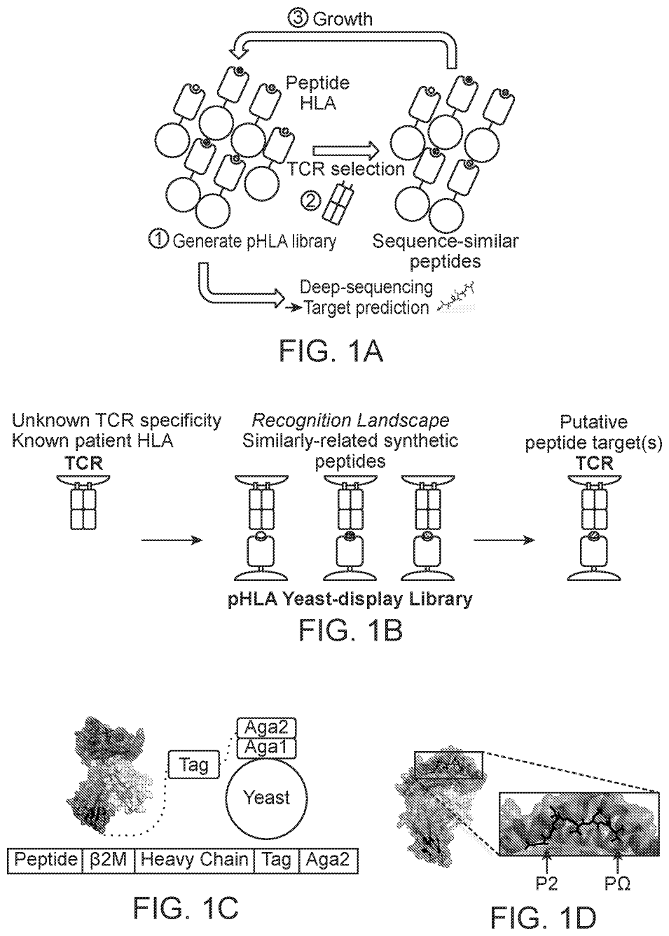

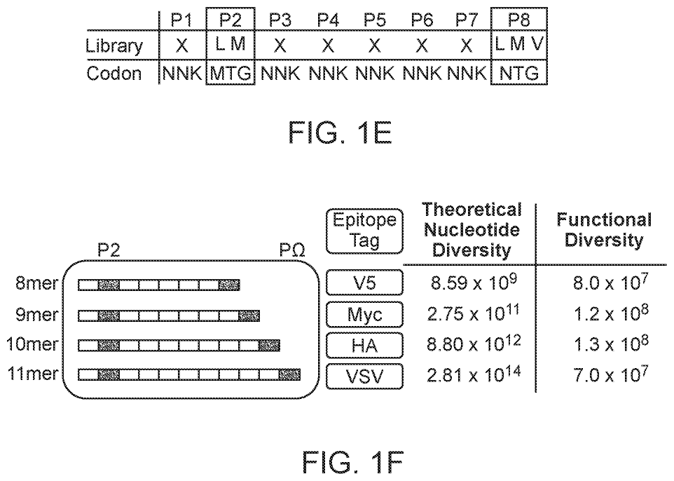

To date, no direct interaction screen or combinatorial display system has been used to determine the antigen specificity of an orphan TCR. Here, we tested our methodology with the goal of identifying antigens recognized by TCRs derived from TILs (FIG. 1B). We applied single-cell T cell phenotyping and TCR sequencing of CD8⁺ TILs in two HLA-A2 homozygous patients with colorectal adenocarcinoma to predict candidate antigen targets from yeast-display library selections (FIG. 1B). Of the TCRs screened, four TCRs isolated peptide targets in the HLA-A*02:01 library. Two of these TCRs were highly similar in sequence and had specificity for an overlapping group of peptides, implying shared antigen specificity. The synthetic peptides isolated from the library, in addition to predicted peptides from the Uniprot human reference genome, stimulated the respective T cell receptors of interest. Surprisingly, three of the four receptors recognized unmutated self-antigens. This serves as proof-of-principle for linking T cell immune responses and their clonal TCRs with a direct antigen identification method using yeast-display libraries. This methodology can serve as a powerful tool to identify novel cancer antigens recognized by the immune response.

Design of the HLA-A*02:01 yeast-display library. The HLA-A*02:01 allele is highly prevalent, present in up to 50% of a number of populations. The binding motifs for peptides presented by HLA-A*02 have been well characterized and a number of restricted clinically relevant TCRs identified. For these reasons, we generated a yeast-display library for screening potential HLA-A*02:01-restricted T cell receptors (FIG. 1A). Individual yeast express a random peptide covalently linked to the HLA molecule, which enables peptide identification by DNA sequencing (FIG. 1C). This pHLA library features an N-terminal peptide library linked to wildtype β-2-microglobulin (B2M) and HLA-A*02:01 heavy chain with a single point mutation Y84A (See STAR Methods). To ensure proper display of peptides in the binding groove, the peptide library restricts amino acid usage at P2 and PΩ to the aliphatic hydrophobic residues preferred by HLA-A*02:01 (FIG. 1D-F). At other positions, NNK codons randomly encode all twenty amino acids to provide an unbiased library. Because HLA-A*02:01 typically presents peptides 8 to 11 amino acids in length, we generated multiple peptide length libraries using epitope tags for multiplexed selections (FIG. 1F). Each library has a theoretical nucleotide diversity dictated by the library composition and length, but the functional diversity of the library is limited (FIG. 1F). In total, we estimate that approximately 400 million unique peptides ranging from 8 to 11 amino acids are represented in the combined libraries.

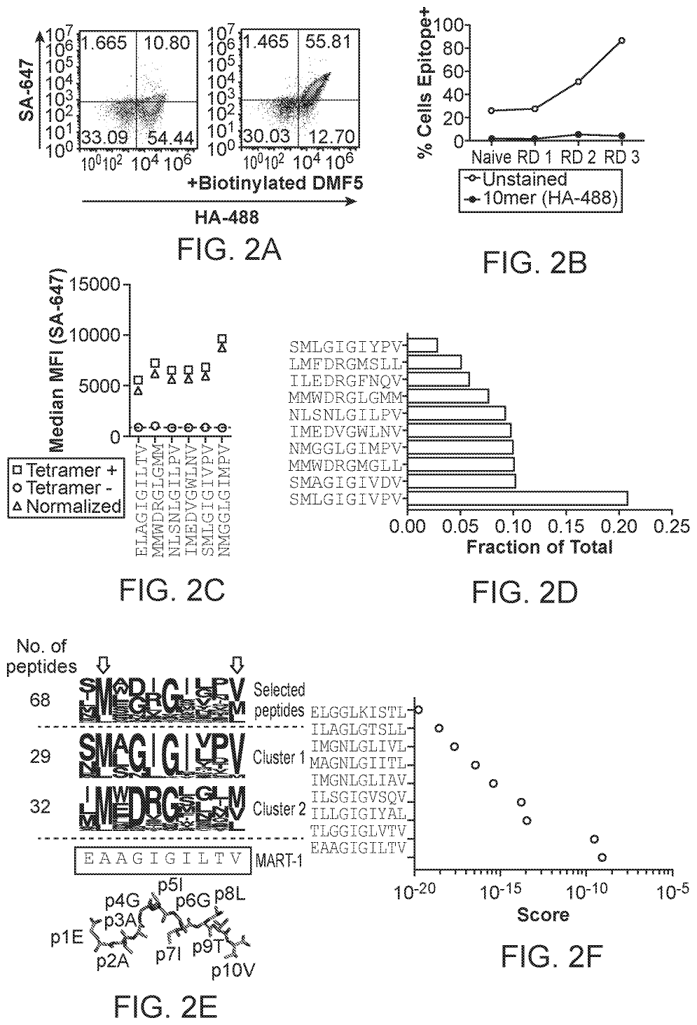

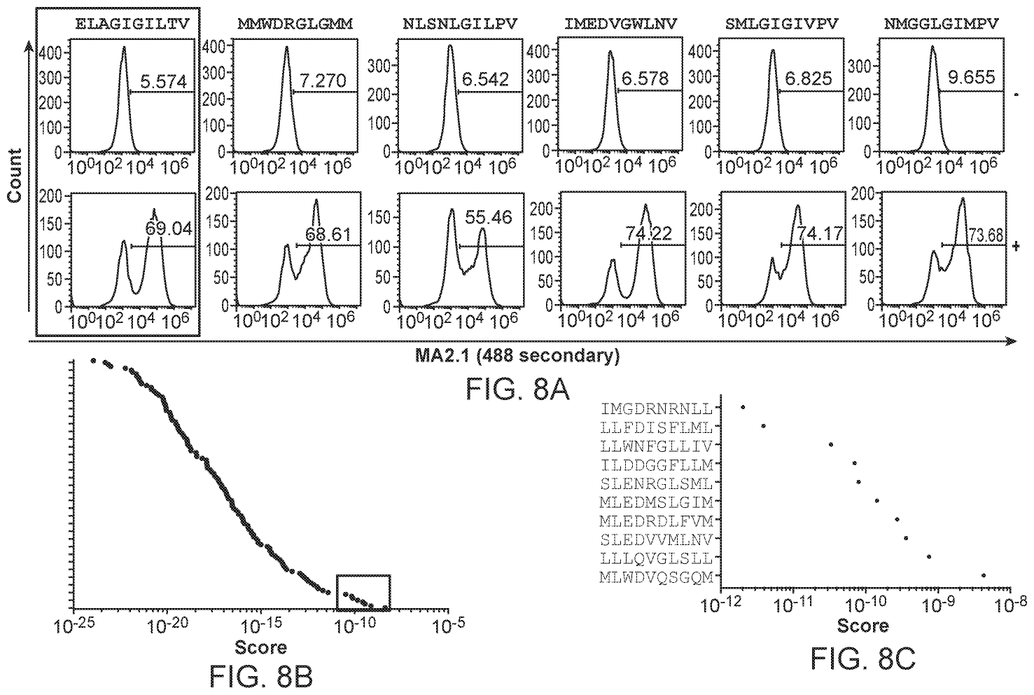

Validation of the library with the MART-1-specific DMF5 TCR. To determine whether the HLA-A*02:01 complex is properly folded to present peptides, we used a ‘proxy’ TCR with known specificity. We used the DMF5 TCR, which is a naturally occurring TCR that recognizes a 10 amino acid sequence (EAAGIGILTV) (SEQ ID NO: 267) derived from the MART-1 melanoma antigen bound to HLA-A*02:01. To validate the HLA-A*02:01 library, the 10 mer heteroclitic peptide ELAGIGILTV (SEQ ID NO: 264), which has improved HLA stability, was displayed with HLA-A*02:01 on yeast and stained by both an anti-hemagglutinin (HA) antibody and 400 nM tetramerized DMF5 TCR, indicating surface expression of the protein complex and proper folding of the pHLA (FIG. 2A). To confirm that the library could be used to identify the antigen of the DMF5 TCR, the HLA-A*02:01 10 mer library (FIG. 1F) was selected by MACS bead-multimerized DMF5 TCR (See STAR Methods, FIG. 2B). A sample of the fourth round of selection was sequenced by Sanger sequencing to identify enriched peptides, most of which were found to be highly related to the MART-1 10 mer peptide (FIG. 2C). Five sequences were individually expressed on the yeast with HLA-A*02:01 and stained with 400 nM DMF5 TCR tetramer to show TCR-specific binding (FIG. 2C) and anti-HLA-A*02 to show conformational expression of the complex (FIG. 8A).

All rounds of the yeast-display selection by the DMF5 TCR were deep-sequenced. The library converged significantly by round 3 of the selection to 68 unique peptides, of which the top 10 peptides dominated 91.7% of the library (FIG. 2D). The most striking observation was that almost all peptides selected had a Gly at P6 (P6G) (Table 1), consistent with the DMF5-MART-1/HLA-A*02:01 crystal structure showing that P6G provides flexibility to allow a cleft for CDR3β 100F, to which P6G hydrogen bonds. Deep-sequencing revealed two major clusters of peptide sequences (FIG. 2E). To clarify these clusters, the reverse hamming distance, which is a metric to identify the number of exact amino acid matches between two peptides, was calculated between all peptides and then clustered by score (FIG. 2E, Table 1). The two major clusters diverged at P4 to P6 with a central ‘GIG’ motif in 29 peptides (cluster 1) and a central ‘DRG’ motif in 32 peptides (cluster 2). Cluster 1 peptides were used in a search matrix to score potential human peptide targets, a method used previously to predict human antigens from yeast-display selection data (2014PWM). However, because the 10 mer library did not allow for Ala at P2 of the library, P2A was manually included in the search matrix matching the anchor with the lowest frequency—Leu at 16.67%. From this analysis, 9 peptides from the human proteome were predicted with varying probabilistic scores to bind the DMF5 TCR (FIG. 2F, Table 1). Strikingly, the human MART-1 peptide was the most probable to bind the DMF5 TCR of the 9 peptides predicted (FIG. 2F). Using cluster 2, orders of magnitude more peptides were predicted to bind the TCR (FIG. 8B, 8C, Table 1). However, the DMF5 TCR has not shown any off-target toxicity, indicating that this other ‘DRG’ peptide motif may not be physiologically relevant in the immune responses of cancer patients in that study.

Blinded validation of the HLA-A*02:01 library with neoantigen-specific TCRs. To test the ability of the HLA-A*02:01 library to identify the antigens of TCRs with unknown antigen specificity, we screened three TCRs derived from a melanoma patient, in which all TCRs had blinded specificities to neoantigens. These antigens had been identified independently by exome sequencing of tumor material, predicting neoantigen presentation by HLA-A*02:01 and staining of patient-derived tumor-infiltrating T cells with peptide-loaded HLA-A*02:01 multimers. The three TCRs, labeled NKI1, NK12, and NK13 were recombinantly expressed and used to select the HLA-A*02:01 library containing all four peptide lengths.

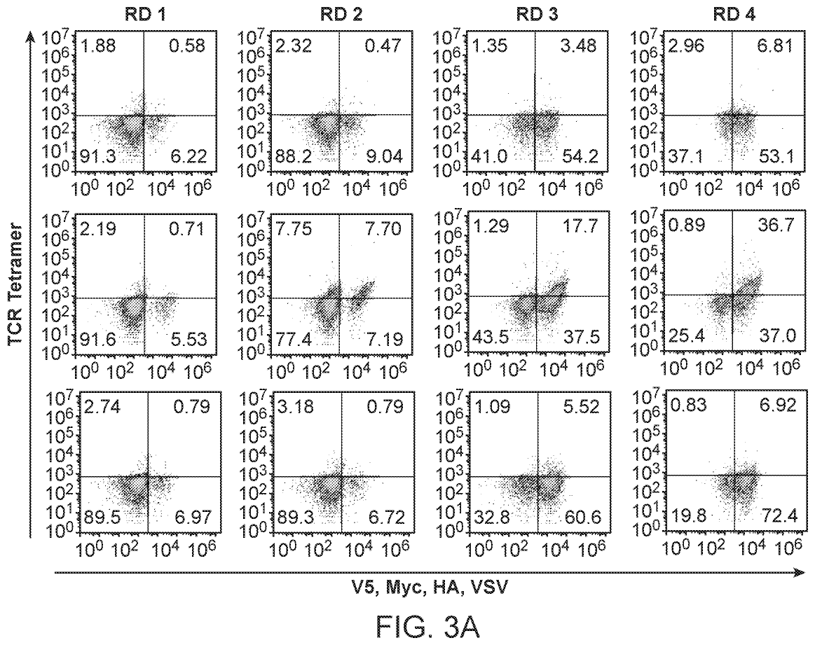

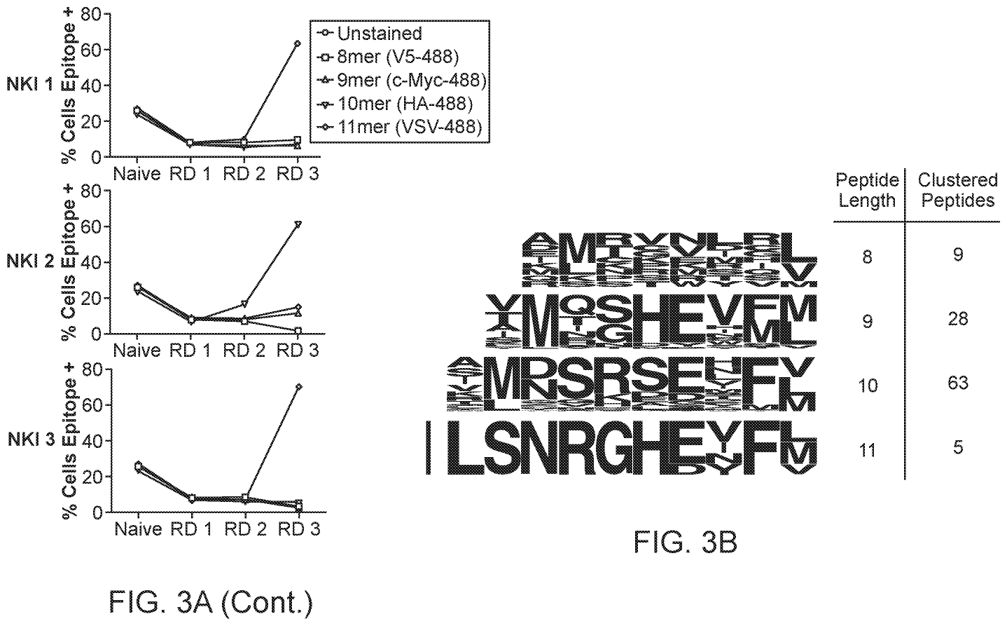

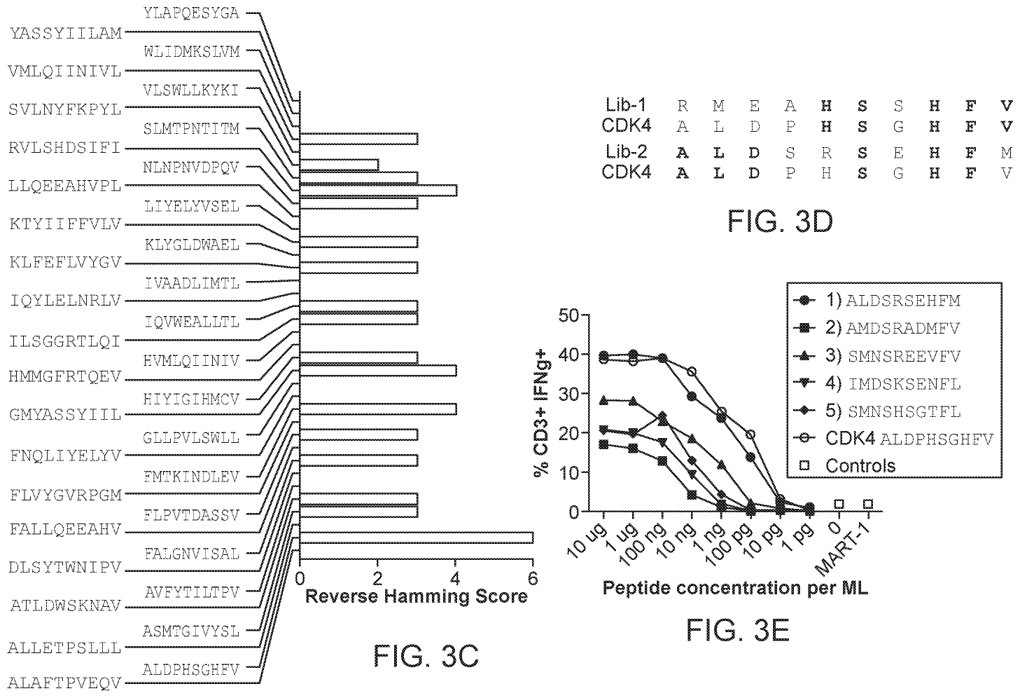

Only the selection for NK12 produced 400 nM tetramer-positive yeast beginning at round 2 of the selection, indicating strong binding of the peptide-HLA-A*02:01 library (FIG. 3A). All rounds of the selection were deep-sequenced, and the data was then parsed based on peptide length per selection round (Table 2). The peptides converged by round 3 of the selection and peptides were clustered by reverse hamming distance (FIG. 3B). The selection results for NK12 showed dramatic similarity in 9 mer, 10 mer, and 11 mer sequences. These peptide sequences share a conserved Glu in the 9 mer, 10 mer, and 11 mer sequences at P6, P7, and P8 respectively, and the peptides share a positively charged residue at P5 of the 9 mer, 10 mer, and 11 mer. NKI1 and NK13 did not produce tetramer-positive selected yeast (FIG. 3A) nor did the deep-sequencing indicate strong peptide selection.

As part of the blinded validation, a list of 127 neoantigens predicted to be presented by HLA-A*02:01 served as candidate ligands for the NKI2 TCR. The reverse hamming distance was calculated for each of these 127 potential neoantigen peptides compared to the list of 10 mer synthetic peptides selected by NKI2 (FIG. 3C). ALDPHSGHFV (SEQ ID NO: 265), a peptide neoantigen derived from cyclin-dependent kinase 4 (CDK4), had 5 and 6 of the 10 positions being identical to library peptides Lib-1 and Lib-2, respectively.(FIG. 3D). CDK4 was correctly identified and confirmed as the neoantigen target of NKI2. The targets of NKI1 and NKI3 could not be unambiguously identified through this blinded validation. NKI1 is specific for the same CDK4 neoantigen and NKI3 is specific for a GCN1L1 neoantigen ALLETPSLLL (SEQ ID NO: 268). Reasons for the lack of target identification are discussed later.

We have established that these synthetic peptides isolated from the pHLA library are specifically recognized by NKI2. We next asked whether they could stimulate either NKI1- or NKI2-expressing T cells. Human peripheral blood lymphocytes were transduced with either NKI1 or NKI2. and co-cultured with HLA-A*02:01⁺ JY cells loaded with each of the top 5 peptides selected by NKI2. Interestingly, all 5 peptides elicited IFNγ production by NKI2 transduced T cells in a dose-dependent manner (FIG. 3F). Furthermore, the top selected peptide mimotope ALDSRSEHFM (SEQ ID NO: 269) stimulated these cells as potently as the CDK4 neoantigen ALDPHSGHFV itself. The 5^{th} most selected peptide by NKI2 stimulated the NKI1 receptor in a dose-dependent manner, indicating overlapping specificities.

Single-cell characterization of tumor-infiltrating lymphocytes in colorectal cancer patients. Our ultimate goal is to identify peptide ligands for TCRs derived from expanded and cytotoxic T cell populations infiltrating patient tumors using the yeast-display platform (FIG. 1B). Single-cell technology for analyzing T cells provides a means to individually phenotype single T cells and to sequence their paired αβ TCRs in a high-throughput manner.

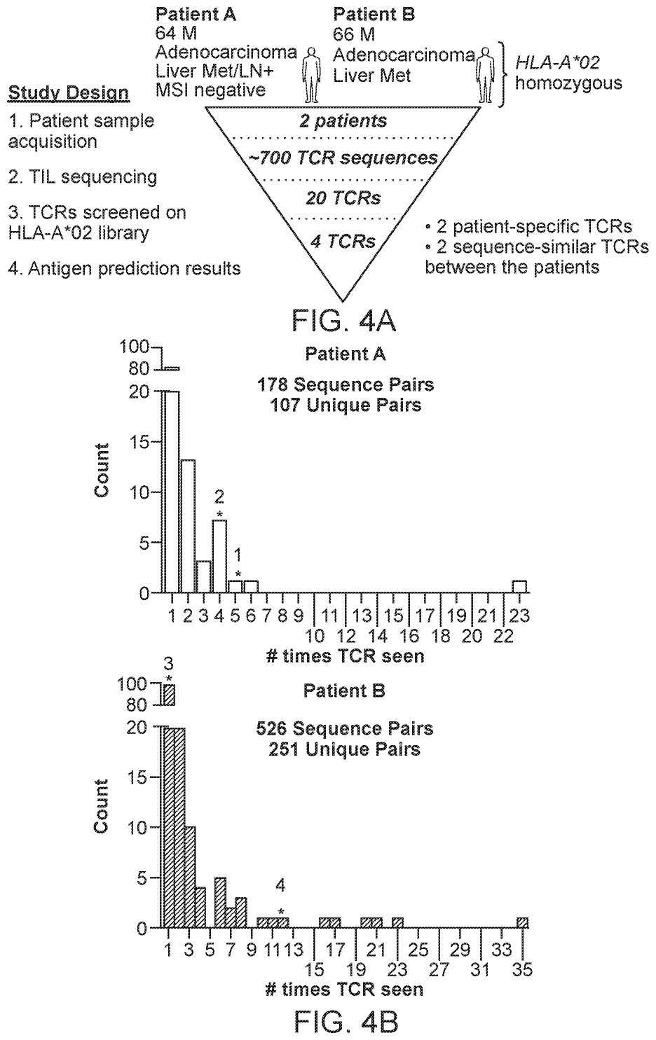

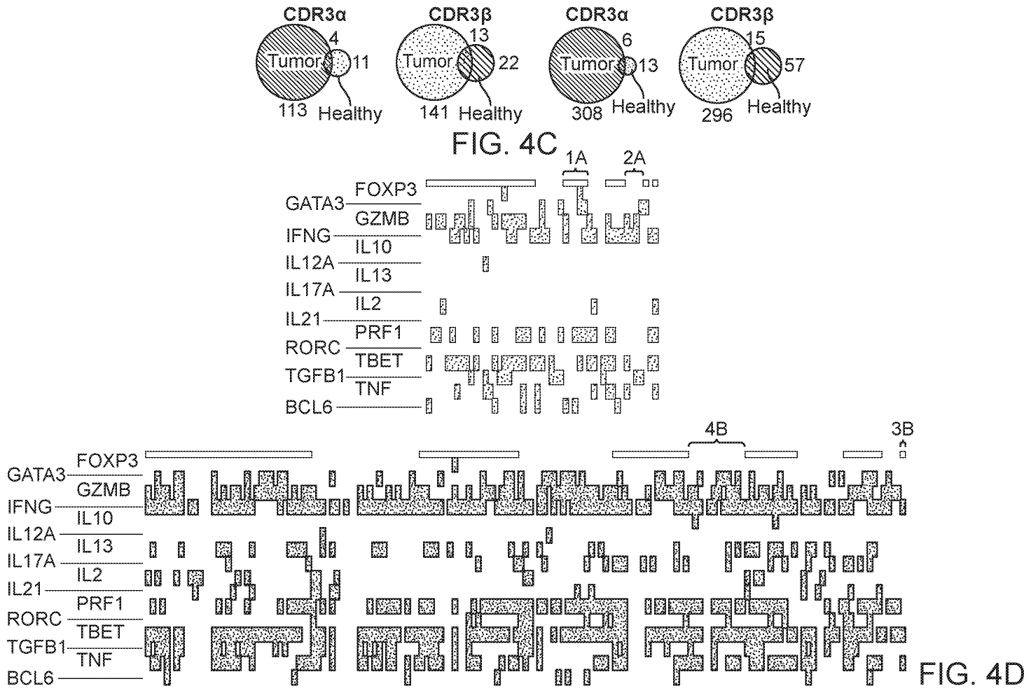

We selected patients homozygous for the HLA-A*02 allele (FIG. 4A). This improves the probability that a T cell isolated from a patient has a receptor restricted to the HLA-A*02 allele; however, it does not exclude the possibility that this TCR may have specificity to other classically or non-classically restricted antigens. The full HLA locus was typed for both patients sans HLA-C (Table 3). HLA-A*02:01 and HLA-A*02:06 differ only by an F9Y substitution in the β-sheet floor which is unlikely to affect TCR recognition. These suballeles have been described to share a subset of presentable peptide antigens, although differences can amount to distinct patterns of TCR multimer staining of pHLA.

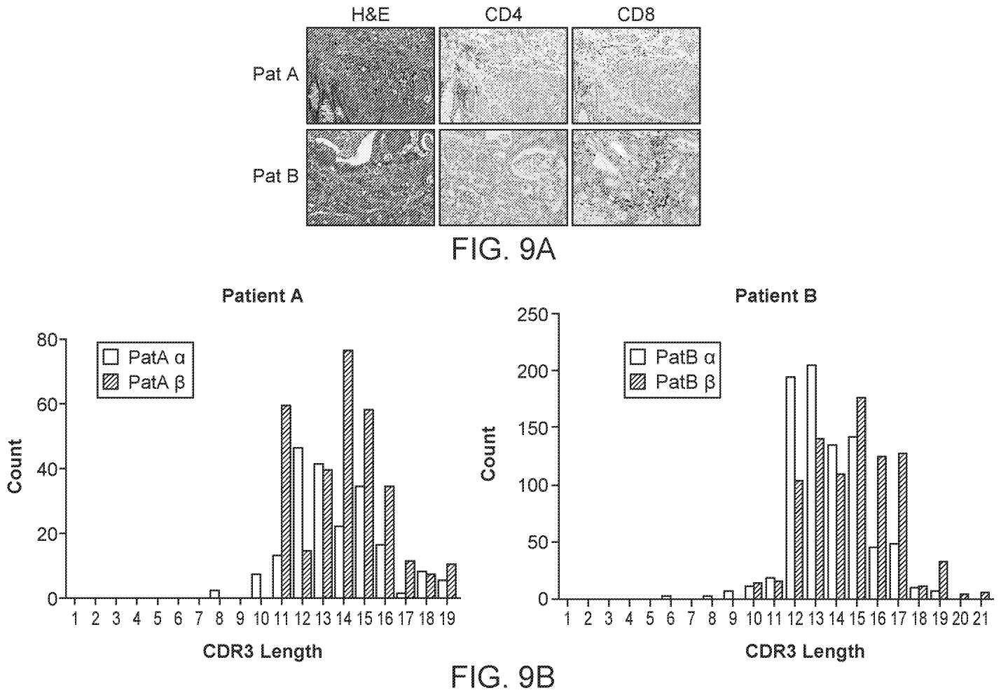

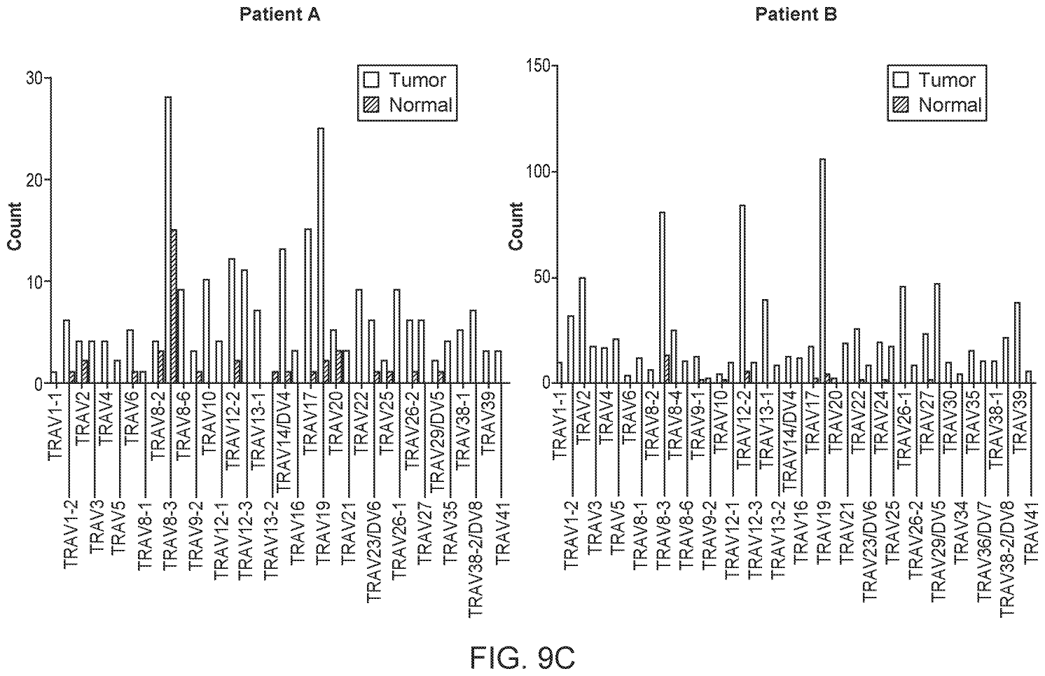

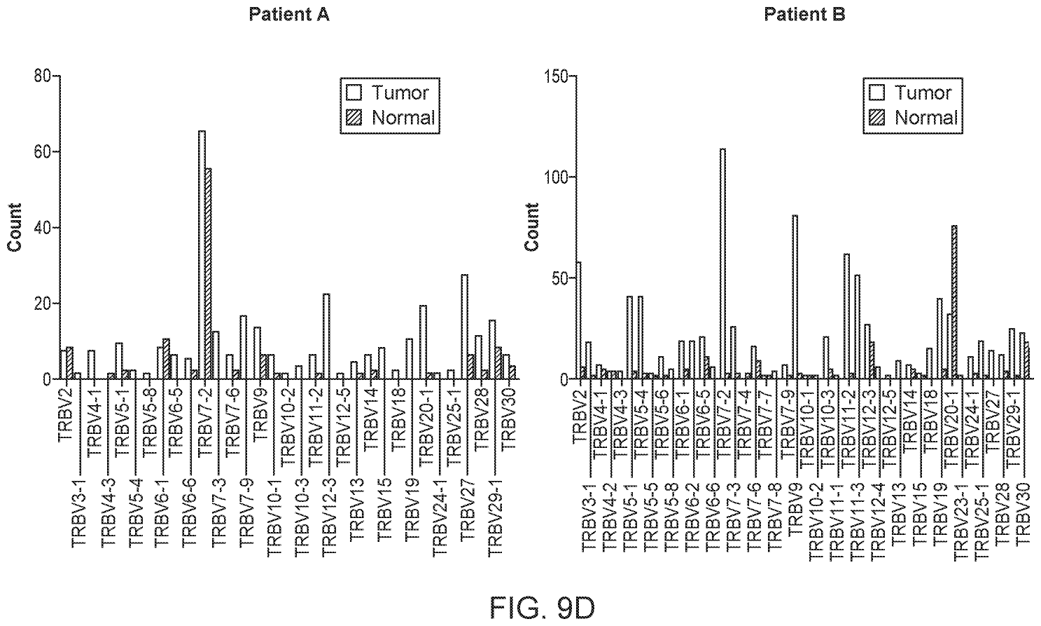

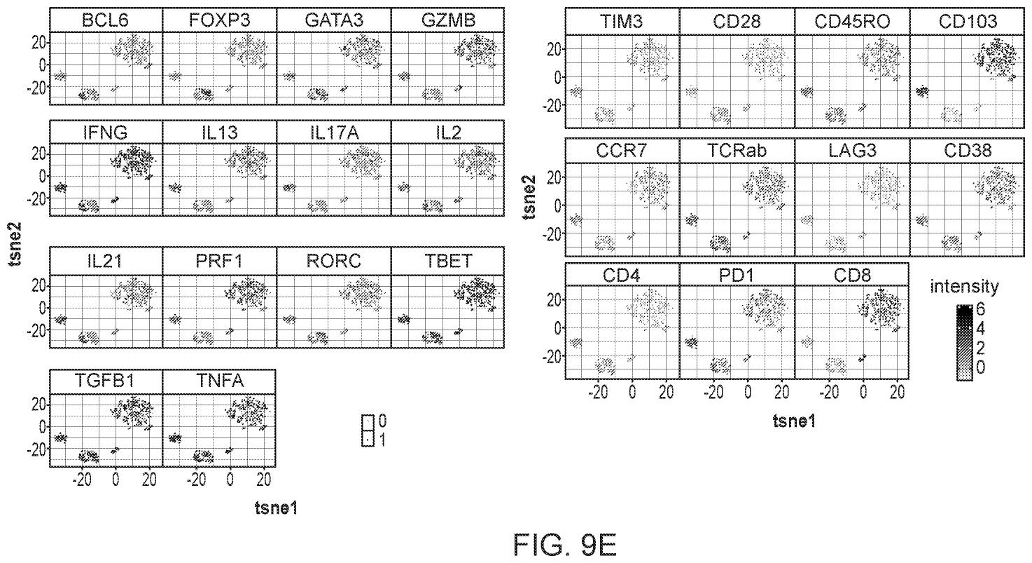

Both patients were males in their mid-60s with colorectal adenocarcinoma (FIG. 4A). Tissue samples of the tumors were analyzed for infiltration of CD8⁺ and CD4⁺ T cells and the overall structure observed by H&E staining (FIG. 9A). For Patient A, CD4⁺ and CD8⁺ T cells were found in the lamina propria of the colon, but less in the tumor. For Patient B, CD4⁺ T cells were not abundant within the colon tissue; however, there was significant CD8⁺ T cell infiltration into the tumor.

From these two patients, several hundred CD8⁺ T cells were phenotyped and sequenced from the site of the tumor with 53-paired sequences from the healthy tissues and 709-paired sequences from the tumor tissues (FIG. 4B). Any clone seen more than once at the site of the tumor is considered an expanded clone. In both cases, there were expanded TCR clones in the tumor, suggesting antigen-specific expansion. The most expanded TCR clones comprised 12.9% (23/178) of the sequenced population in Patient A and 6.67% (35/526) in Patient B, respectively. This level of expansion at the tumor is consistent with other reports of T cell repertoire populations in primary liver carcinoma and CD4+ T cells infiltrating colorectal carcinoma. Because not many T cells were identified from healthy tissue, clones were considered exclusive to the tumor and not shared with healthy tissue if either α or β chain are not shared. For both patients, both α and β chain sequences showed only a small overlap of sequences between tumor and healthy tissues (FIG. 4C). This suggests that most TIL T cell clones are enriched and present in the tumor as a result of tumor-driven responses; however, we cannot conclude that any TIL TCR is exclusively present within tumor due to limited sampling of healthy tissue.

The T cell receptors sequenced from the patients exhibited typical CDR3α and CDR3β lengths (FIG. 9B). Both patients had a predominance of TRAV8-3, TRAV19 (FIG. 9C), and TRBV7-2 (FIG. 9D) expression. Unlike T cells from Patient A, T cells from Patient B were analyzed by index sorting, allowing for pairing of cell surface marker expression and transcript expression. When separating T cell populations based on cell surface markers and transcriptional profiles using t-Distributed Stochastic Neighbor Embedding (t-SNE), CD8⁺ and CD4⁺ T cell populations separated into major clusters (FIG. 9E). For Patient B, there was significant CD8⁺ T cell infiltration into the tumor and the majority of cells sampled co-expressed PD-1 and IFN₁ with a heterogenous expression of other cytotoxic markers granzyme B, perforin, and TNF-α. It has been suggested that the PD-1⁺ CD8⁺ T cell population is the tumor-reactive population.

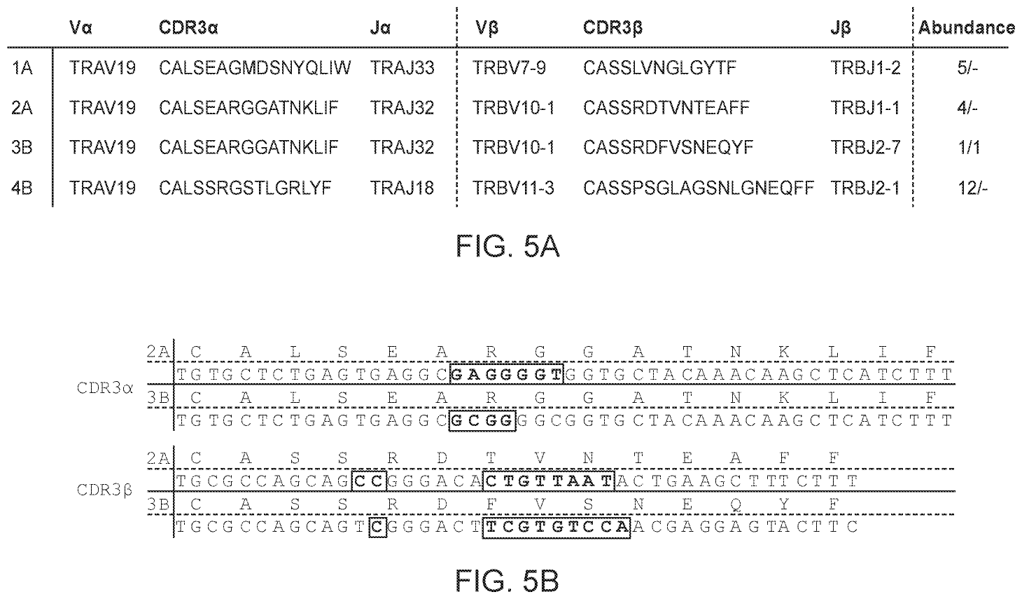

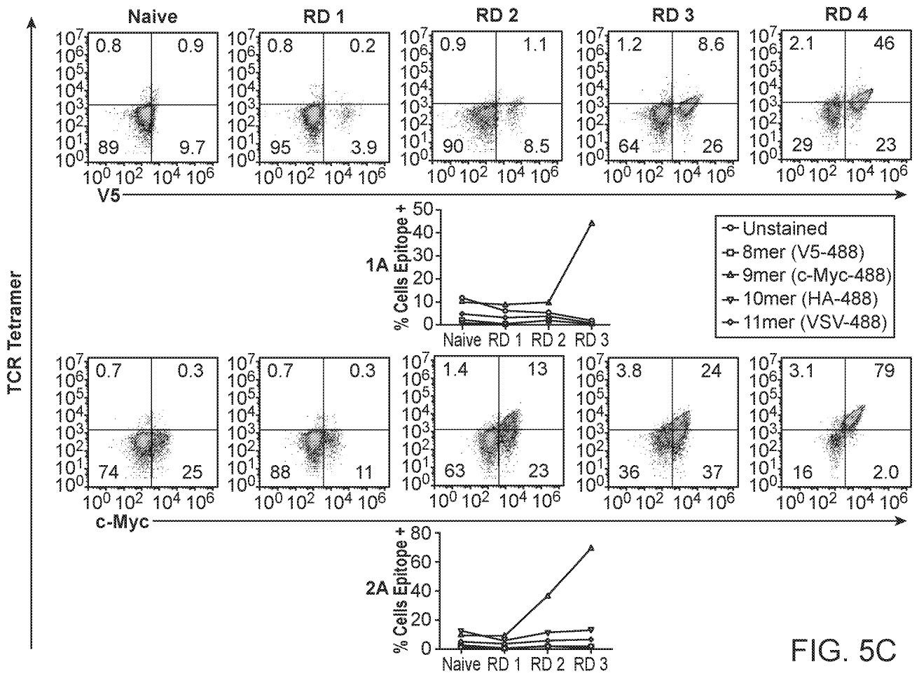

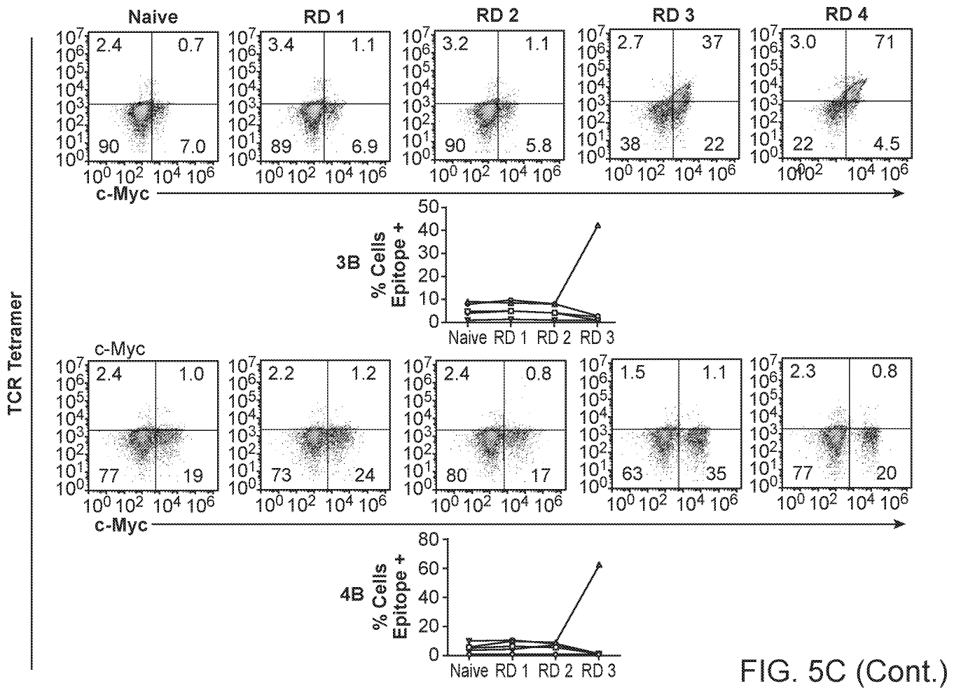

Screening Orphan TCRs on the HLA-A* 02:01 Library. Twenty candidate receptors were chosen based on local expansion at the tumor, cytotoxic profile (IFN₁, TNFα, perforin, granzyme B), and in some cases based on common TCR chain usage (FIG. 4B, 4D). Of the twenty candidate TCRs (Table 4) screened on the HLA-A*02:01 library, four TCRs enriched peptides from the library, TCRs 1A and 2A derived from Patient A and TCRs 3B and 4B derived from Patient B (FIG. 5A). Interestingly, two receptors, 2A and 3B, isolated from separate patients, express the same TCRα chain and similar TCR chains, which contain CDR3β sequences of the same length with five conservative amino acid differences and a central Val residue completely generated by NP addition (FIG. 5B).

Each TCR was screened on the HLA-A*02:01 library. Each of the four TCRs enriched an HLA-linked epitope tag expressed by the yeast, while the remaining sixteen TCRs did not (FIG. 5C). For TCRs 1A, 2A, and 3B, tetramer stained yeast gradually increased across the rounds of selection. However, TCR 4B did not stain the yeast despite successive enrichment of the 9 mer epitope tag (FIG. 5C). A reason for the lack of enrichment of the remaining sixteen TCRs screened is most likely HLA restriction to alternative HLA alleles with other possibilities explored in the discussion.

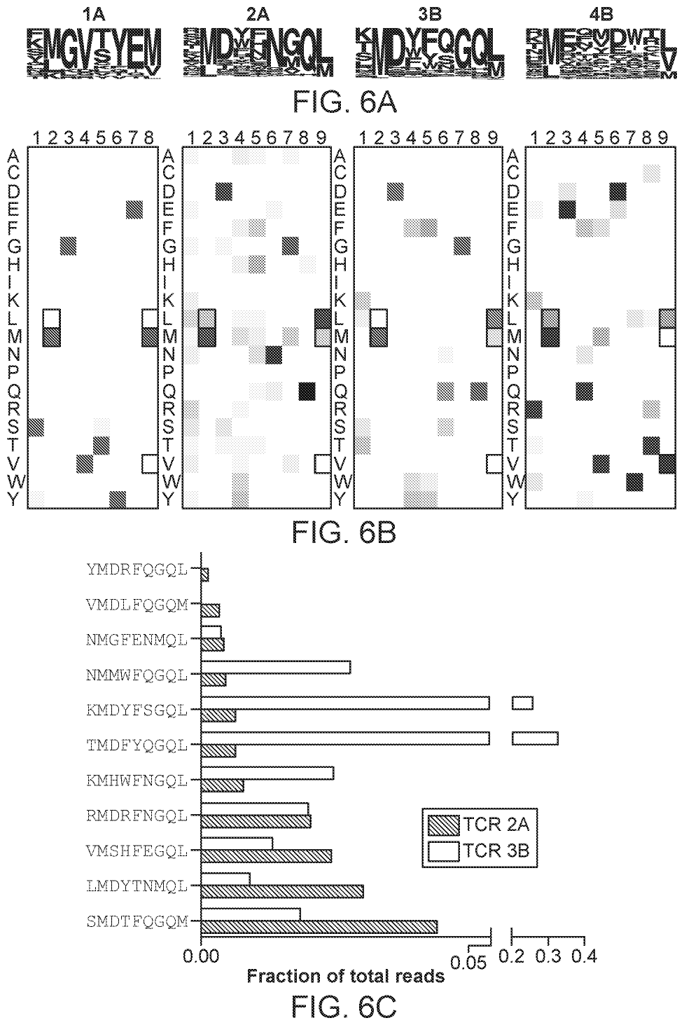

The yeast selected by TCRs 1A, 2A, 3B, and 4B were deep sequenced (Table 4). For all four TCRs, sequences converged by round 3 of the selection and the unique peptide sequences were used to generate peptide motifs to identify positional hotspots (FIG. 6A). The highly similar TCRs 2A and 3B selected for related peptide sequences, 11 of which were common to both (FIG. 6C). The selection of a common pool of peptides suggests that these TCRs recognize the same antigen. However, significant differences are seen between these two motifs at P6 with an invariant Asn for TCR 2A and Asn, Glu, and Ser predominant for TCR 3B. In general, TCR 2A displays a wider degree of cross-reactivity selecting 190 unique peptides with positions P1, P4, and P5 allowing more amino acid substitutions than in the 66 unique peptides selected by TCR 3B. TCRs 1A and 4B have different motifs entirely with 15 and 61 unique peptides selected, respectively at the third round of selection.

One method to measure cross-reactivity of a T cell receptor is to observe the selected breadth of tolerated amino acids at a particular position of the peptide. To do this, we determined the proportions of all amino acids at every position, accounting for peptide enrichment at round 3 (FIG. 6B). TCR 1A and 3B are relatively specific for their peptide motif with more rigidity in amino acid preference per position. In contrast, TCRs 2A and 4B are more cross-reactive in their specificity, allowing degeneracy at positions along the peptide, except for the limited anchor residues. Despite the close similarities in amino acid sequences between 2A and 3B, the TCRs display a high contrast in cross-reactivity for their peptide landscapes. In this respect, the pHLA library screening is effective at ‘measuring’ the relative cross-reactivity of TCRs, which could be important for selection of TCRs for adoptive cell therapy, in which limited cross-reactivity may be desired to limit autoreactivity.

TCR target prediction from hu man proteome and patient exomes. The peptides identified in the yeast-display selections generate a recognition landscape of sequences for each TCR. As was done for the DMF5 TCR using the 2014PWM, this information can be used in an algorithm to predict stimulatory human antigens. In applying the algorithm to the colorectal cancer data, we generated human predictions for TCR 2A, but yielded no predictions for TCR 1A and TCR 3B and limited predictions for TCR 4B. This motivated the development of two additional methods to predict human peptides from selection data—a modified variant of the previous statistical method (2017PWM) and a method utilizing a two-layer convolutional neural network (2017DL) (See STAR Methods). Data from previous selections using the DR15 library was used to test the accuracy of the 2017PWM and 2017DL algorithms in predicting peptide antigens. MBP was the best prediction using 2017DL and the second best prediction using 2017PWM for TCR OB1.A12 and the second best prediction in both algorithms for TCR OB1.2F3.

The additional two algorithms were used to score predicted peptides from the human proteome using the UniProt database. For TCRs 2A and 3B, there were many peptides that were predicted by multiple algorithms for both TCRs, indicating shared target specificity. Overall, the three algorithms were able to collectively make predictions from the human proteome for all four TCRs.

Because patient mutations can generate neoantigens recognized by T cells, we performed exome sequencing and variant calling to identify potential candidates. In total, 762 PASS variants were identified in Patient A and 4,763 PASS variants identified in Patient B with at least 30× sequencing coverage for both healthy and tumor tissue. Exome peptides were scored by the 2017PWM and 2017DL algorithms, but very few were significant across the TCRs. One exception was a 21-nucleotide translocation from an intron to exon 7 of the same WDR66 gene, which generated a neoantigen peptide in Patient A, albeit with sub-optimal HLA anchors that would result in it being poorly presented, if at all. This resulted in a novel peptide sequence EYGVSYEW (SEQ ID NO: 270), which closely matches the peptide motif for patient A-derived TCR 1A. Overall, the predictions for the four TCRs suggest that three of the four are likely to bind unmutated self-antigens.

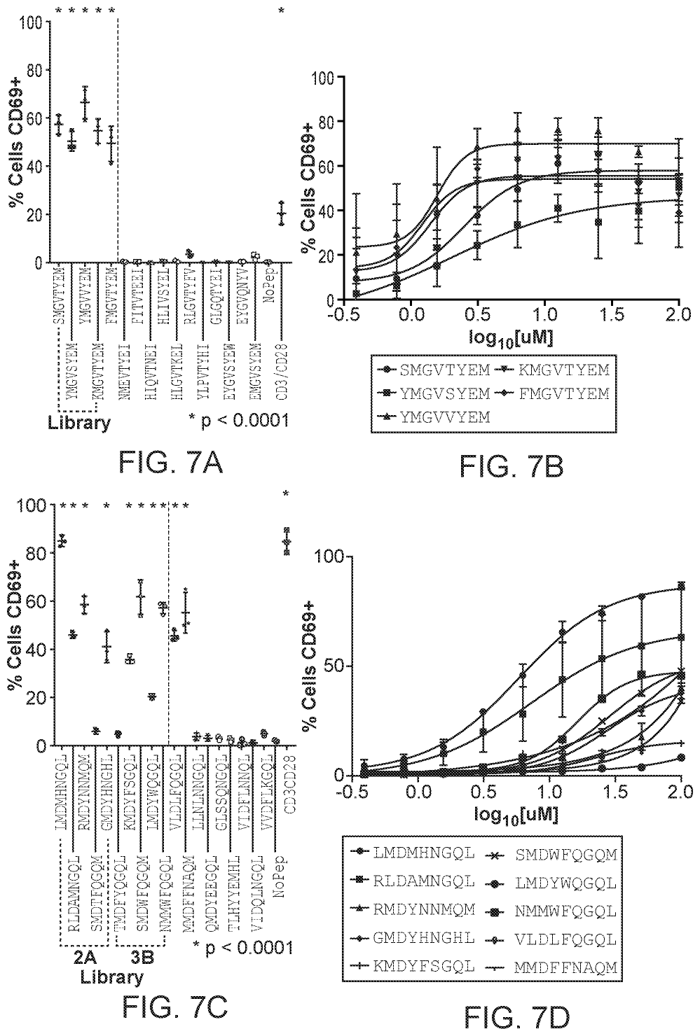

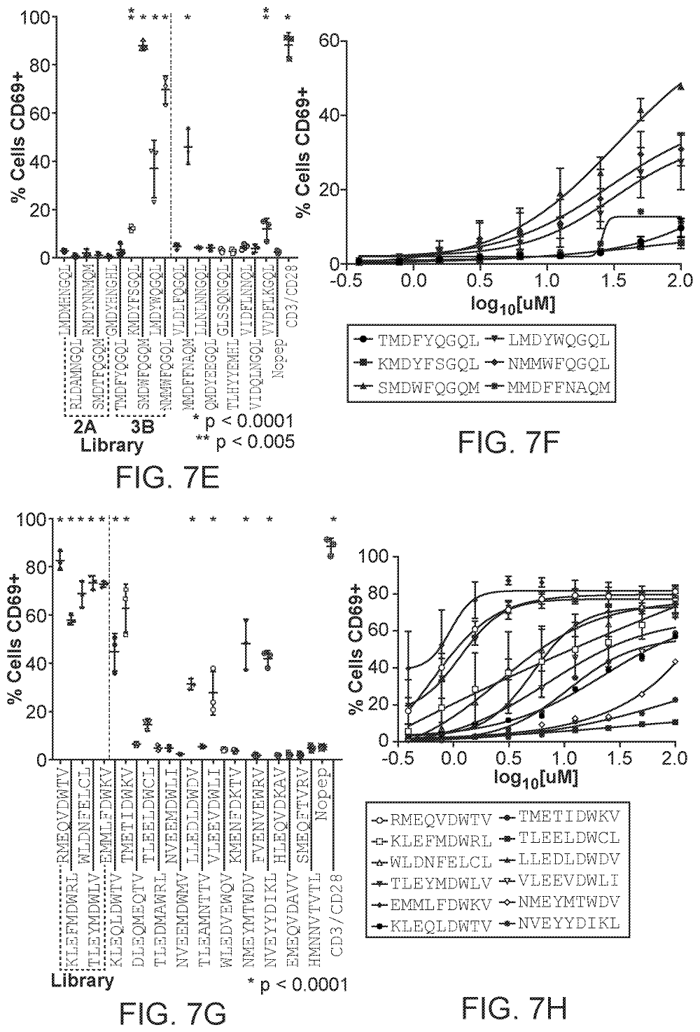

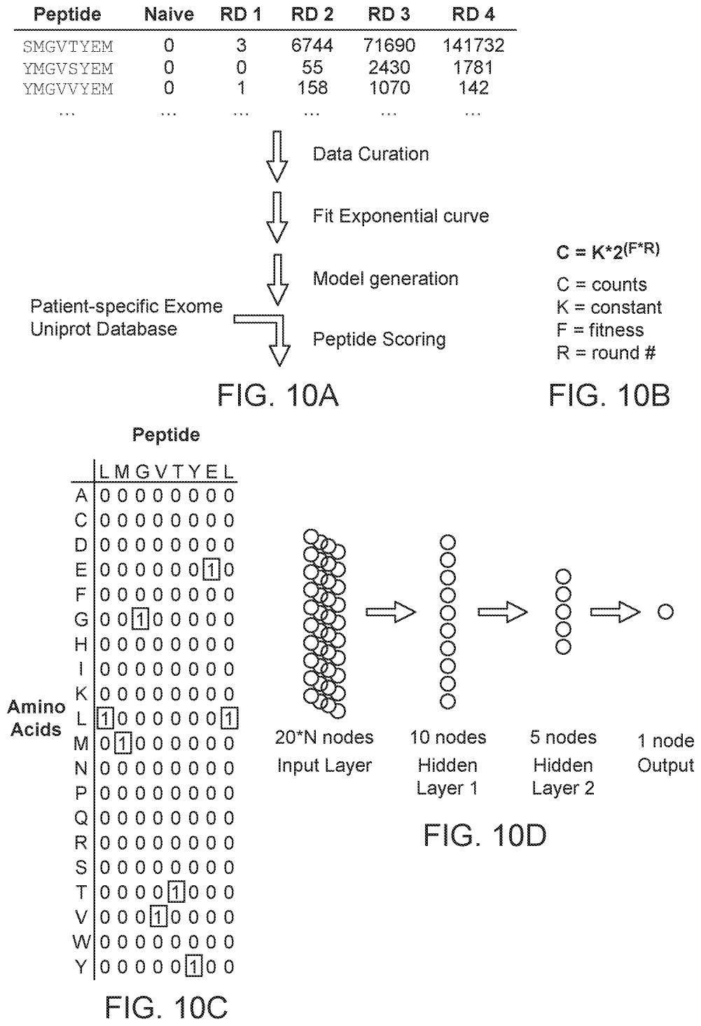

In vitro target validation of synthetic and predicted human peptides. Both synthetic peptides selected from the library and the predicted human peptides from the human and/or exome were presented by T2 cells used to stimulate SKW-3 CD8⁺ T cell lines modified to express the four TCRs identified from the patients. Interestingly, the synthetic library peptides selected by TCR 1A all potently stimulated the T cells via CD69 activation (FIG. 7A, FIG. 10A) and in a dose-dependent manner (FIG. 7B). For TCR 1A, the exome peptide (EYGVSYEW) (SEQ ID NO: 270), the anchor-modified exome peptide (EMGVSYEM) (SEQ ID NO: 271), nor the human peptide predictions stimulated the cell line (FIG. 7A). Although we have identified a strong antigen recognition motif for TCR 1A, we have not been able to recover a stimulatory endogenous antigen, only mimotopes.

For the three TCRs 2A, 3B, and 4B (FIG. 7C-H), we were able to identify stimulatory endogenous antigens. TCR 4B was stimulated by its selected synthetic peptide libraries and also stimulated by 6/19 of the predicted human peptides, which is in accord with the higher degree of cross-reactivity seen in the yeast selection deep-sequencing analyses (FIG. 7G, 7H, FIG. 10D). Interestingly, we see that TCR 4B is stimulated by antigens from two different putative driver genes WDR87₁₃₁₀₋₁₃₁₈ (peptide LLEDLDWDV) (SEQ ID NO: 272), a testis-expressed antigen found to be recurrently mutated in colorectal cancer, and CRISPLD1₈₂₋₉₀ (peptide NMEYMTWDV) (SEQ ID NO: 273), a protein expressed in many cancers with no known function. The cysteine-rich secretory proteins, antigen 4, and pathogenesis-related 1 proteins (CAP) superfamily includes CRISPLD1, and these proteins have been implicated in a wide-range of functions including ion channel regulation, reproduction, cancer, cell-cell adhesion, and others. From exome analysis, Patient B has a mutation in CRISPLD1 at D143Y. TCR 4B is also stimulated by 5 other human antigens including CD74₁₈₁₋₁₈₉ peptide TMETIDWKV (SEQ ID NO: 274), FANCI₁₁₀₄₋₁₁₁₂ peptide VLEEVDWLI (SEQ ID NO: 275), GEMIN4₇₇₁₋₇₇₉ peptide KLEQLDWTV (SEQ ID NO: 276), PDE4a₂₄₃₋₂₅₁ peptide TLEELDWCL (SEQ ID NO: 277) or PDE4b₂₃₁₋₂₃₉ peptide TLEELDWCL (SEQ ID NO: 277), and KLHL7₅₀₆₋₅₁₄ peptide NVEYYDIKL (SEQ ID NO: 278). The true in vivo specificity cannot be unambiguously identified without additional tumor information.

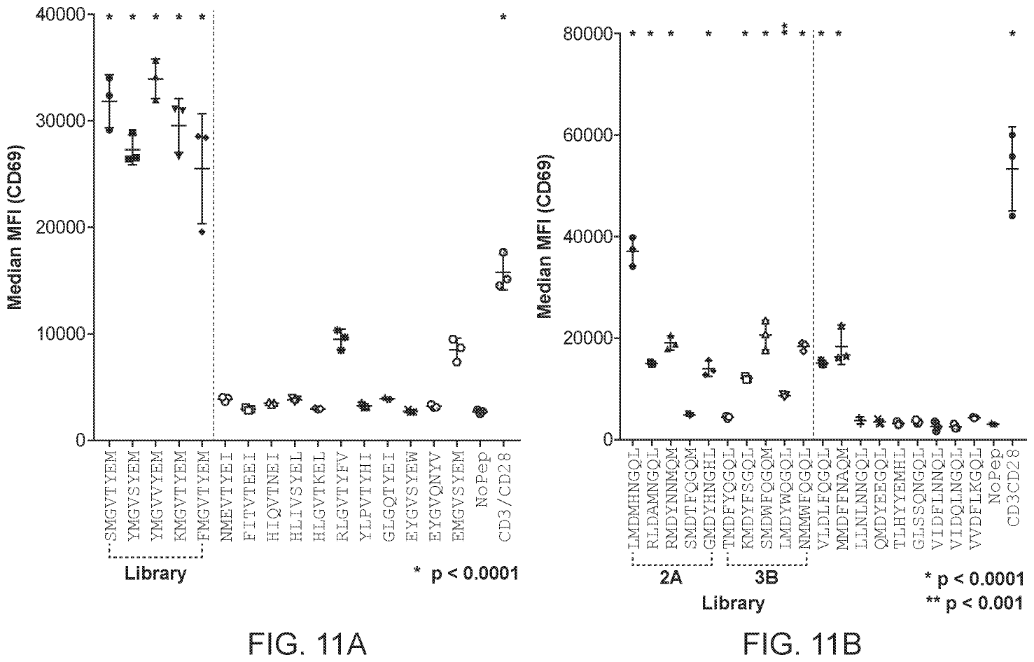

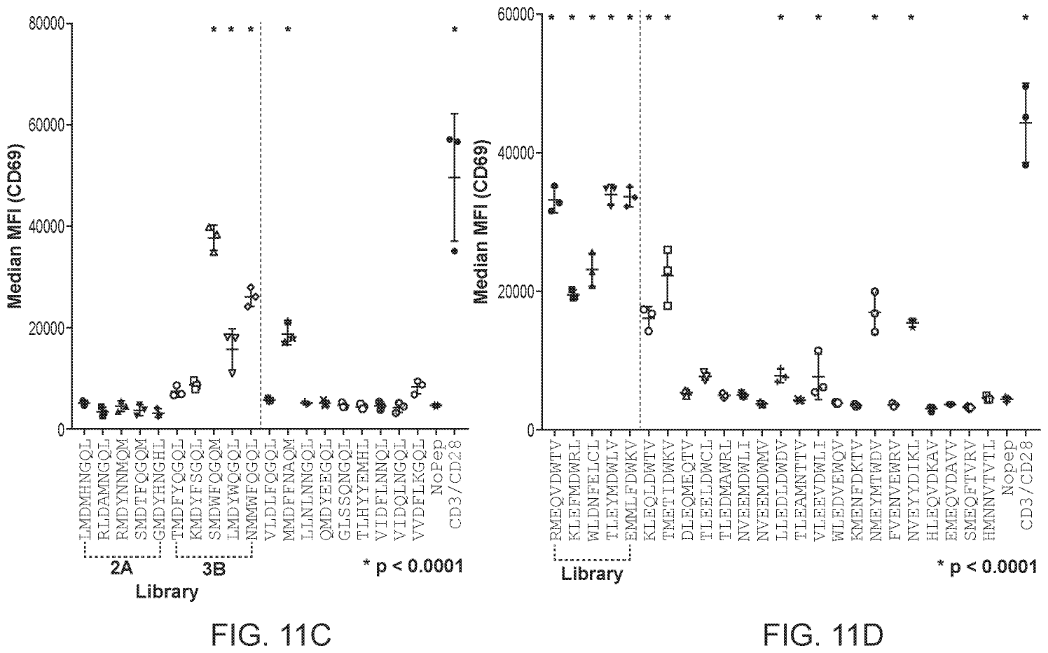

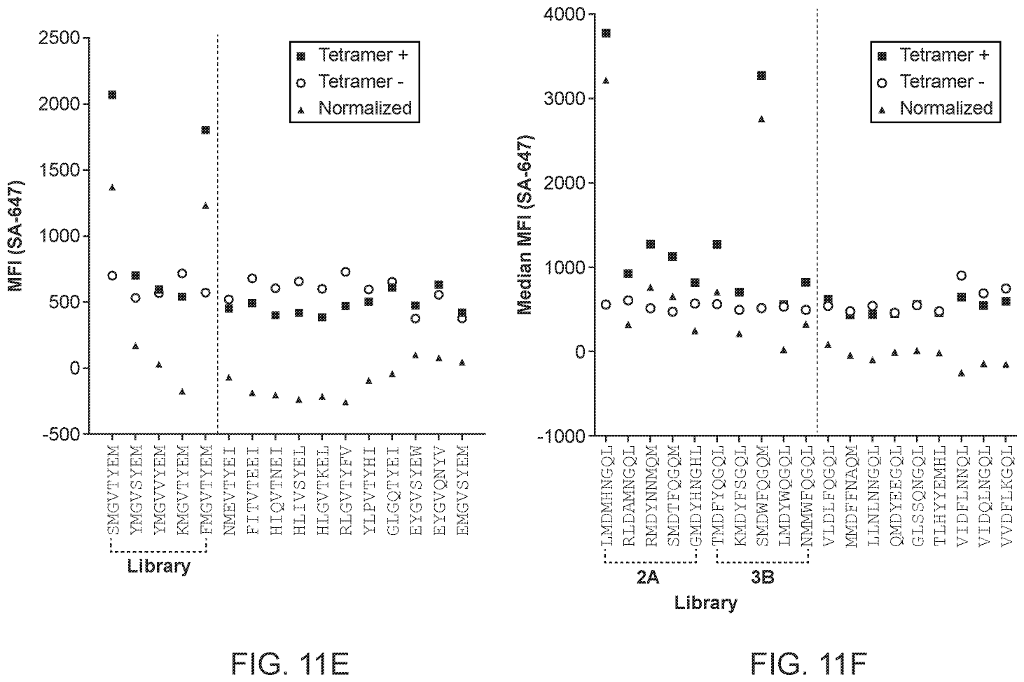

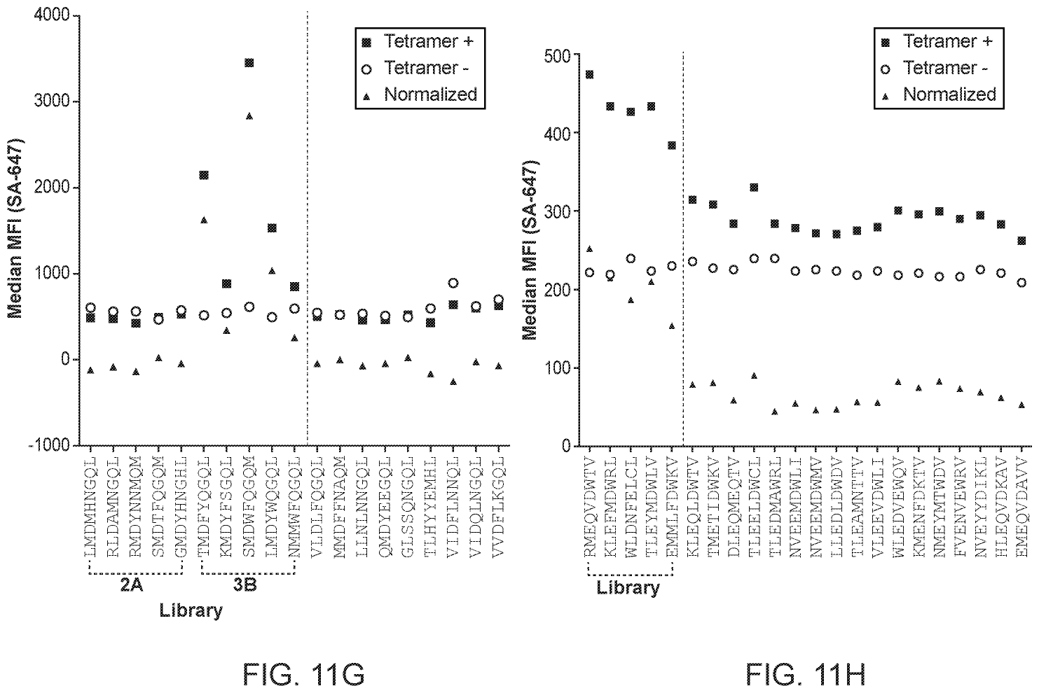

The highly similar TCRs 2A and 3B have different stimulatory profiles against the selected synthetic peptides (FIG. 7C-F, FIG. 10B-C). TCR 2A cells were stimulated by four of the top five peptides selected by TCR 2A and four of the top five peptides selected by TCR 3B. However, TCR 3B cells were only stimulated by four out of the top five peptides selected by its own TCR and none selected by TCR 2A. These results support the finding that TCR 3B is relatively selective compared to TCR2A (FIG. 6B). Strikingly, of the 26 human peptides tested from the predictions (Table 6), only a single human peptide was found to stimulate T cells with bearing either receptor (FIG. 6C, 6E). This peptide is MMDFFNAQM (SEQ ID NO: 279), which is derived from U2AF2₁₇₄₋₁₈₂, a protein involved in an RNA splicing complex. U2AF2 is normally expressed in many human tissues and overexpressed in many cancers including colorectal cancer as determined by antibody staining deposited in the Protein Atlas. In fact, U2AF2 RNA was overexpressed in tumor tissue over healthy tissue by 2.11- and 2.65-fold in Patient A and Patient B, respectively (FIG. 11A). When examining human lymphoma, breast, colon, and lung tumor cell lines, U2AF2 RNA is overexpressed significantly relative to patient samples (FIG. 11B-C). U2AF2 has been implicated in promotion of tumor metastasis in melanoma and is rarely mutated in chronic myelogenous leukemia, myelodysplastic syndromes, and solid tumors like lung adenocarcinomas. U2AF1, U2AF2's binding partner, is commonly mutated in cancer and mutations have shown enhanced RNA splicing and exon skipping, leading to gene dysregulation in vitro. In both patients, no mutations were found in U2AF2 or U2AF1. For the more cross-reactive TCR 2A compared to TCR 3B, an additional human peptide (SEQ ID NO:280) VLDFQGQL derived from protein TXNDC11₁₀₇₋₁₁₅ was able to stimulate the receptor, which has not been previously described to be involved in cancer, but is expressed in the colon and many other tissue types.

We determined by surface plasmon resonance the affinity of TCR 2A for the peptide MMDFFNAQM (SEQ ID NO: 279) displayed by HLA-A*02:01 to be 110 μM, identifying a bona fide interaction (FIG. 11D-E). An affinity could not be determined for TCR 3B. These low affinities may explain, in part, the lack of TCR tetramer staining of yeast expressing the single-chain MMDFFNAQM-HLA-A*02:01 (SEQ ID NO: 281) (FIG. 10F-G). These discordant results of stimulation versus tetramer binding are seen across all TCRs studied (FIG. 10E-H). Conversely, MMDFFNAQM-HLA-A*02:01 (SEQ ID NO: 281) tetramers failed to stain SKW-3 cells expressing either TCR2A or TCR 3B. Unfortunately, tissue samples were not available to confirm peptide presentation by HLA-A*02 by mass spectrometry. Although we cannot definitively determine an immune response targeting the peptide derived from U2AF2, the evidence from the yeast-display screen, prediction algorithm, and in vitro stimulation identify this peptide as the likely target. These results serve as proof-of-principle that pHLA libraries can identify the antigen specificity of TCRs, having identified a shared specificity across two patients. The pHLA libraries can also correctly distinguish relative cross-reactivities for peptide antigens.

The fundamentally surprising insight from our studies is that the specificity encoded in the small recognition kernel of the MHC-bound peptide visible to the TCR is sufficient to enable reconstruction of entire sequences of endogenous peptides to TCRs of unknown specificity. This finding has important implications for the identification of antigens in T cell mediated diseases. T cells provide an avenue of therapeutic treatment in infectious diseases, autoimmunity, allergy and cancer. In most of these, we have very little information about T cell specificities, especially in humans, because of limited methods. This situation has advanced by the availability of high-throughput methods to obtain TCR sequences from single T cells directly ex vivo, but one is still faced with the daunting task of determining peptide ligand(s). Here we combine a single cell TCR analysis method with a refined version of the yeast display library screening approach to discover novel pHLA specificities in human colorectal adenocarcinoma. This has broad implications for our understanding of T cell specificities in cancer and can be applied to other diseases.

To our knowledge, this is the first instance of TCR ligand identification using a combinatorial biology screening technology, in which three TCRs were found to be specific for wildtype antigens, which have roles in cancer. A single wildtype antigen derived from U2AF2 is likely a shared immune response target in 2/2 patients studied. For all TCRs that were successfully screened on the HLA-A*02 library, we were able to identify multiple mimotope peptides that stimulated these TCRs, often more potently than the native peptide. Akin to neoantigens, the synthetic peptide antigens or mimotopes have utility as DNA, RNA or peptide vaccines to stimulate particular antigen-specific T cells and generate a more immunogenic response than the self-antigen that the immune response is likely tolerant towards.

The success of predicting the cognate tumor antigen from deep sequencing selection data depends on improved and refined search algorithms and patient tissue validation. Additionally, screening large numbers of TCRs from a given tumor can increase the odds of linking selection data to the cognate antigen, especially when coupled to relevant patient data including RNA expression and/or mass spectrometry of eluted peptides.

Two principal applications are available for this method in immunotherapy: 1) to identify endogenous and mimotope ligands for orphan TCRs and/or 2) as a means of classifying TCRs based on peptide antigen specificities, which will allow the identification of clinical candidate TCRs that recognize shared antigens across patients. Shared TCRs can either be receptors that share similar TCR sequence, which can potentially lead to shared antigen specificity, or TCRs that do not have any shared sequence but recognize the same antigen. Such TCRs recognizing shared antigens would be especially useful in engineered T cell or vaccine therapies. As TCR sequencing continues to advance and more TCR sequencing data becomes available, we can infer TCR restriction for patient HLA and infer a common TCR specificity for convergent TCR sequence clusters. This enables TCR ligand identification to be more effectively directed at impactful TCRs with known HLA restriction.

Unlike other methods utilizing exome data to identify patient-specific neoantigens that can serve as potential targets of the T cell immune response, this method is an unbiased interrogation of TCR specificities of the present immune response that relies on a physical interaction between the TCR and pHLA. This ligand identification method may be especially important in cancers that have low mutational burden, in which neoantigen targets may not be as prevalent compared to wildtype antigens. We have developed a methodology improving upon the use of yeast-display libraries to de-orphanize TCRs that can provide a means for identifying clinically important TCRs and novel antigens. We have validated the HLA-A*02:01 library as a tool for de-orphanization of TILs in two patients with colorectal adenocarcinoma. We predominantly identified wildtype antigens as targets of these patient immune responses, with a shared response to a wildtype antigen of potential therapeutic value.

### STAR Methods

### Experimental Model and Subject Details

Human Subjects. Two male subjects of age 64 and 66, both with colorectal adenocarcinoma. The Stanford University Institutional Review Board approved all protocols for collection of human tissue and blood. Patient samples were obtained with patient consent from the Pathology Department at Stanford Hospital. Both patients were HLA typed sans HLA-C and specifically chosen for their HLA-A*02 allelic expression.

Primary and Cell Lines. All cells are grown at 37° C. with 5% CO₂ unless otherwise stated.

Human PBMCs were cultured in RPMI complete (ThermoFisher) containing 10% fetal bovine serum (FBS), 2 mM L-glutamine (ThermoFisher) and 50 U/mL penicillin and streptomycin (ThermoFisher). SKW-3 cells are derived from a human T cell leukemia and cultured in RPMI complete containing 10% FBS, 2 mM L-glutamine, and 50 U/mL penicillin and streptomycin. Transduced cells are cultured with additional 1 ug/mL puromycin (ThermoFisher) and 20 ug/mL zeocin (ThermoFisher). T2 cells are HLA-A*02 positive cells used as antigen-presenting cells to SKW-3 cells. They were cultured in IMDM (ThermoFisher) with 10% FBS, 2 mM L-glutamine, and 50 U/mL penicillin and streptomycin. JY cells are EBV-immortalized B cell line cultured in RPMI complete containing 10% FBS, 2 mM glutamine, and 50 U/mL penicillin and streptomycin. HEK 293T cells are grown in DMEM complete (ThermoFisher) containing 10% FBS, 2 mM L-glutamine, and 50 U/mL penicillin and streptomycin. FLYRD18 are grown in DMEM complete with 10% FBS with 2 mM glutamine with 50 U/mL penicillin and streptomycin.

EBY100 yeast cells are grown in either SDCAA, which contains 20 g dextrose, 6.7 g Difco yeast nitrogen base (BD Biosciences), 5 g Bacto casamino acids (BD Biosciences), 14.7 g sodium citrate (Sigma-Aldrich), 4.29 g citric acid monohydrate (Sigma-Aldrich) per liter of H₂O at pH 4.5 or SGCAA, which replaces dextrose with galactose. The yeast are grown at 30° C. in SDCAA or 20° C. in SGCAA for protein induction at atmospheric CO₂.

High Five cells are grown in Insect X-press media (Lonza) with final concentration 10 mg/L of gentamicin sulfate (ThermoFisher) at 27° C. at atmospheric CO₂. SF9 cells are grown in SF900-III serum-free media (ThermoFisher) with 10% FBS and final concentration 10 mg/L of gentamicin sulfate at 27° C. at atmospheric CO₂

Preparation and selection of y east-display libraries. Yeast-display libraries were generated as previously reported (Birnbaum et al., 2014) using chemically competent EBY100 yeast (ATCC). In short, primers encoding chosen codon sets were used to generate DNA-encoded peptide libraries. Anchor positions at P2 and PΩ of the peptide has limited codon usage to Leu-Met and Leu-Met-Val, respectively, while NNK codon diversity was allowed at all other positions (FIG. 1E, Table 8). Separate length libraries encode different length codon sets and vectors used unique epitope tags for multiplexed selections: 8 mer—V5 tag, 9 mer—myc tag, 10 mer—HA tag, 11 mer—VSV tag. To display the peptide/HLA*A-02:01 complex on the yeast, the heavy chain of the HLA*A-02:01 was modified with Y84A mutation and the heavy chain truncated at S302. This mutation allows an opening for a linker to thread between the C-terminal end of the peptide, through the end of the peptide binding groove, to B2M to generate a single-chain trimer. The transmembrane-truncated heavy chain is linked to an epitope tag linked to the Aga2p protein for yeast-display. The diversities of the yeast libraries were determined post-electroporation by colony counting after limiting dilutions.

Yeast were mixed at 10× diversity of the individual length libraries and frozen at −80° C. in 2% glycerol and 0.67% yeast nitrogen base. Libraries were thawed as needed in SDCAA pH 4.5, passaged, induced in SGCAA, and subsequently selected as described previously (Birnbaum et al., 2014) using biotinylated soluble TCR coupled to streptavidin-coated magnetic MACS beads (SAb) (Miltenyi). In short, 10× diversity of yeast containing all four length libraries (4×10⁹ cells) were negatively selected with 250 μL SAb for 1 hr at 4° C. in 10 mL of PBS+0.5% bovine serum albumin and 1 mM EDTA (PBE). Yeast were passed through an LS column (Miltenyi) attached to a magnetic stand (Miltenyi) and washed three times. The flow through was then incubated for 3 hr at 4° C. with 250 μL SAb pre-incubated with 400 nM biotinylated TCR for 15 minutes at 4° C. Once again, yeast were passed through an LS column and the elution was grown in SDCAA pH 4.5 overnight after an SDCAA wash. Once yeast reached an OD>2, they were induced in SGCAA with 10% SDCAA for 2-3 days before an additional selection. All subsequent selections were done using 50 μL SAb or TCR-coated SAb in 500 μL of PBE. The fourth round was done using a negative selection following a 1 hr incubation of yeast with 400 nM SA-647 in 500 uL PBE followed by a PBE wash and an incubation with 50 μL of anti-Alexa647 Microbeads (Miltenyi) for 20 minutes. The positive selection was done after a 3 hr incubation with 400 nM SA-647 TCR tetramer followed by 20 minutes of anti-Alexa647 Microbeads for 20 minutes. The naïve library and all rounds of selection were processed for deep-sequencing as described below. Each round was monitored post-induction with anti-epitope staining and 400 nM TCR tetramer staining completed at 4° C. for 3 hrs.

Individual yeast clones isolated from the selections or competent yeast electroporated with reconstructed peptide-HLA constructs identified from the deep sequencing were stained with 400 nM TCR tetramer labeled with SA-647 or SA-647 alone in combination with anti-epitope tag.

Deep sequencing of pHLA libraries. DNA was isolated from 5×10⁷ yeast per round of selection by miniprep (Zymoprep II kit, Zymo Research). Individual barcodes and random 8 mer sequences were added to the flanking regions of the sequencing product by PCR and amplified for 25 cycles (Table 8). These primers amplified from the signal peptide of the construct to mid-sequence of the B2M. This was followed by an additional PCR amplification adding the Illumina chip primer sequences to generate final products containing Illumina P5-Truseq read 1-(N₈)-Barcode-pHLA-(N₈)-Truseq read 2-IlluminaP7. The library was purified by agarose gel purification, quantified by nanodrop and/or BioAnalyzer (Agilent Genomics), and deep sequenced by Illumina Miseq sequencer using a 2×150 V2 kit for a low-diversity library.

Expression of soluble TCR. Each chain of the F5 TCR was expressed separately in E. coli BL21 (DE3) and purified, refolded, and functionally validated. For all other TCRs, each chain of the TCR was expressed separately using SF9 cells to produce baculovirus in the pAcGP67a vector (BD Biosciences). Both the α and β chain contained the gp67 signal peptide corresponding to the TCR Vα or TCR Vβ. Both constructs utilized a polyhedrin promoter expressing the TCR V region with human constant regions truncated at the connecting peptide for soluble expression and with an engineered disulfide (Boulter et al., 2003). Both chains either expressed a C-terminal acidic GCN4 zipper-6× His tag or a C-terminal basic GCN4 zipper-6× His tag. All chains containing the acid zipper contained the biotinylation acceptor peptide. Both chains contained a 3C protease site between the C-terminus of the TCR ectodomains and the GCN4 zippers. The DNA was co-transfected into SF9 cells with BD baculogold linearized baculovirus DNA (BD Biosciences) with Cellfectin II (Life Technologies). Viruses were generated in 2 mL cultures. Viruses were passaged at dilution of 1:1000 in 25 mL cultures at 1×10⁶ cells/mL to generate more potent virus, which was then co-titrated in 2mL of High Five (Hi5) (ThermoFisher Scientific) cells at 2×10⁶ cells/mL to generate dilutions for 1:1 expression of TCR a and β chains by SDS-PAGE gel and coomassie staining. Co-titrations ranged from 1:1000 to 1:250 for each chain.

Virus was used to infect Hi5 cells for protein expression in 1 to 4 L volumes at 2×10⁶ Hi5 cells/mL. Cells were removed 2-3 days post-infection and supernatant treated to 100 mM Tris-HCl pH 8.0, 1 mM NiCl₂, and 5 mM CaCl₂ to precipitate contaminants. Precipitants were removed by centrifugation and supernatant incubated for 3 hrs with Ni-NTA resin (Qiagen) at room temperature. Protein was washed with 20 mM imidazole in 1× HBS pH 7.2 and then eluted in 200 mM imidazole in 1× HBS pH 7.2. Protein was biotinylated overnight with birA ligase, 100 uM biotin, 40 mM Bicine pH 8.3, 10 mM ATP, and 10 mM Magnesium Acetate at 4° C. after buffer-exchange to 1× HBS pH 7.2 in a 30 kDa filter (Millipore). Protein used for surface plasmon resonance was treated with 3C protease (10 ug/mg of TCR) O/N. Protein was purified by size-exclusion chromatography using an AKTAPurifier (GE Healthcare) Superdex 200 column (GE Healthcare). Fractions were isolated, run on SDS-PAGE gel to confirm 1:1 stoichiometry and biotinylation by streptavidin shift. Fractions were pooled and TCRs were quantified by nanodrop and frozen at −80° C. for storage in 1× HBS buffer pH 7.2.

The Stanford University Institutional Review Board approved all protocols for collection of human tissue and blood. Patient samples from two males aged 64 and 66 were obtained with patient consent from the Pathology Department at Stanford Hospital. A portion of tumor tissue sample was processed by formalin-fixed paraffin embedding for immunohistochemical staining. Tissue was stained used anti-CD4 (clone 1F6, Leica biosystems), anti-CD8 (clone C8/144b, Dako), or hematoxylin/eosin. Fresh tumor and healthy samples were processed as previously done (Han et al., 2014). In short, tumor tissue was divided and incubated with 10 MM EDTA in PBS for 30 min. Cell suspensions were made and passed through a 10-μM nylon cell strainer (Becton Dickinson) and treated with 0.5 mg/mL Type 4 collagenase for 30 min (Worthington Biochemical) in RPMI with 5% FBS. Tissue was disrupted with a blunt-ended 16-gauge needle and syringe. Some samples were saved for antibody staining to isolate tumor tissue by staining for EpCam (clone 9C4, Biolegend) and LIVE/DEAD Fixable Dead Cell Stain kit (Invitrogen) and sorted by FACS using ARIA II (Becton Dickinson) to be processed by AllPrep DNA/RNA Mini Kit (Qiagen) for DNA/RNA extraction. Otherwise, lymphocytes were enriched by Percoll (GE Healthcare) gradient centrifugation and cells frozen in RPMI containing 10% dimethylsulfoxide and 40% FBS or used immediately for antibody staining. Lymphocytes were pre-stimulated non-specifically for 3 hours using 150 ng/mL PMA+1 μM ionomycin prior to staining for FACS. Cells were washed with PBS+0.05% sodium azide+2 mM EDTA+2% FCS.

Lymphocytes were stained with the following antibodies: anti-CD4 (RPA-T4, BioLegend), anti-CD8 (OKT8, eBiosciences), anti-aβ TCR (IP26, BioLegend), anti-TIM3 (F38-2E2, BioLegend), anti-CD28 (CD28.2, Biolegend), anti-CD103 (Ber-ACT8, BioLegend), anti-CCR7 (G043H7, BioLegend), anti-LAG3 (3DS223H, Invitrogen), anti-CD38 (HIT2, BioLegend), anti-CD45RO (UCHL1, BioLegend), and anti-PD1 (EH12.2H7, BioLegend). Dead cells were excluded using a LIVE/DEAD Fixable Dead Cell Stain kit (Invitrogen). Cells were sorted by fluorescence-activated cell sorting (FACS) using an ARIA II (Becton Dickinson) directly into One-Step RT-PCR buffer (Qiagen). Patient B samples were analyzed by index sorting. Reactions were amplified using pooled primer sets as generated previously (Han et al., 2014), barcoded, and pooled for purification by agarose gel purification and deep-sequenced by Illumina Miseq using the 2×250 V2 kit. Data was processed using a custom software pipeline and individual wells were called for CDR3, TCRα and TCRβ variable, joining, and diversity regions using VDJFasta. Data was analyzed using t-SNE based on T cell transcriptional markers and phenotypic markers to separate cell populations.

Sequencing and variant calling of patient exomes. The DNA extracted from tumor and healthy tissue was used to generate libraries for exome sequencing. DNA of 50 ng from tumor and normal tissue were made into Illumina sequencing libraries using Nextera (Illumina). Libraries were pooled and enriched for exonic regions using Roche Nimblegen SeqCap EZ 3.0 (Roche). Paired-end 75 bp reads were generated using a Nextseq500. Tumor-specific variants were determined following GATK Best Practices. Briefly, adapters and low quality bases were trimmed using cutadapt v1.9. Reads were aligned to hg19 using BWA MEM 0.7.12. Duplicates were removed using Picard tools v1.119 followed by indel realignment and base recalibration using GATK v3.5 and reference files downloaded from the GATK Resource Bundle 2.8. Median coverage was determined using bedtools v2.25.0. Lastly, variants between normal and tumor were determined using mutect2. Manufacturer's instructions were followed in all kits and default software parameters were used in all pipelines.

All exome variants were used to generate alternate coding sequences using the Grch37 assembly from Ensembl. Each alternate coding sequence was processed and scored based on the length of the library peptide. Peptides were scored using the 2017PWM and 2017DL algorithms.

Developing algorithms and predictions for human peptides. Deep sequencing results were analyzed as done previously (Birnbaum et al., 2014) with a modification to incorporate deconvolution of the library for different peptide lengths. Different length peptides were identified based on the number of amino acids flanked by the signal peptide and GS linker. In short, paired-end reads were determined from the deep sequencing results using PandaSeq. Paired-end reads are parsed by barcode using Geneious version 6 to identify the round of selection. All nucleotide sequences with less than 10 counts in rounds 3 and 4 of the selection and which differed by only 1 nucleotide sequence from another sequence in the round were coalesced to the dominant sequence. Any data with frameshifts or stop codons were removed from further analysis. Sequences were processed using custom perl scripts and shell commands.

Reverse hamming distances are hamming distances subtracted from the total length of the peptide, representing the number of shared amino acids between two peptides. They were calculated using Matlab (Mathworks Inc.) by iterating through each peptide against all other peptides from the selected round 3 library sequences. The output score generated is the number of matching amino acid positions between peptides. Based on the reverse hamming distances, peptides were clustered using Cytoscape and cutoffs determined manually based on peptide similarity. For the DMF5 TCR, clustering was done and clusters were used to generate substitution matrices for predictions using no cutoff for amino acid frequencies. For the NKI TCRs, the reverse hamming distance was sufficient for determining the neoantigen specificity for the NK12 TCR. The 2014PWM model did not yield any prediction results from the list of 127 neoantigens. Clustering was not done for the four colorectal cancer-derived TCRs prior to algorithm prediction.

For 2014PWM and 2017PWM, substitution matrices were generated from round 3 of all the selections and used to search human protein (Uniprot) or patient-specific exomes to score peptides of fixed lengths using a sliding window. Substitution matrices are made by determining the frequency of all amino acids per position of the peptide. For all predictions made using the 2014PWM except for those made for the DMF5 TCR, a cutoff of 0.1% frequency for an amino acid at a given position was instituted to remove noise. The scores of the peptides are calculated as the product of amino acid frequencies at each position. The 2017PWM is less stringent than the 2014PWM, in that it allows predicted peptides to incorporate amino acids at positions not found in the selected peptides of the library. This prevents discarding peptide sequences that may not have been selected for, but could potentially be a viable peptide solution.

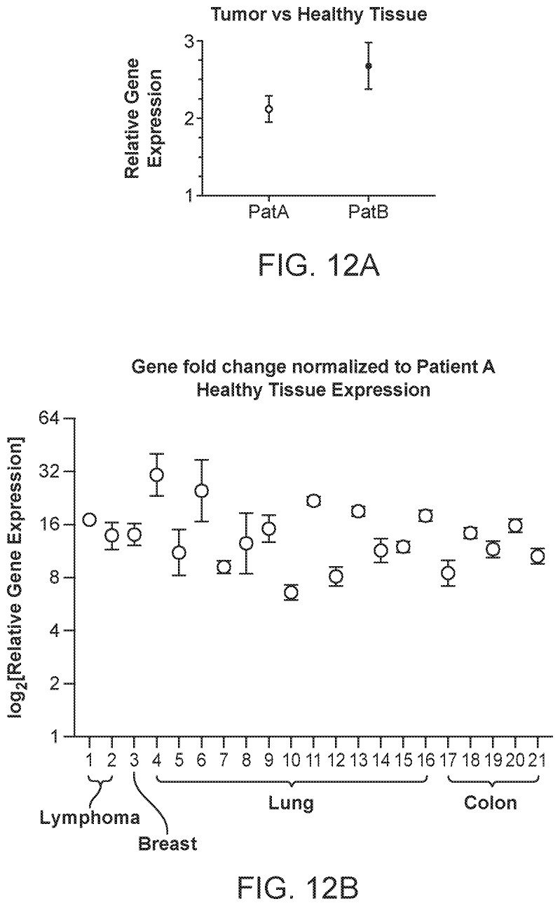

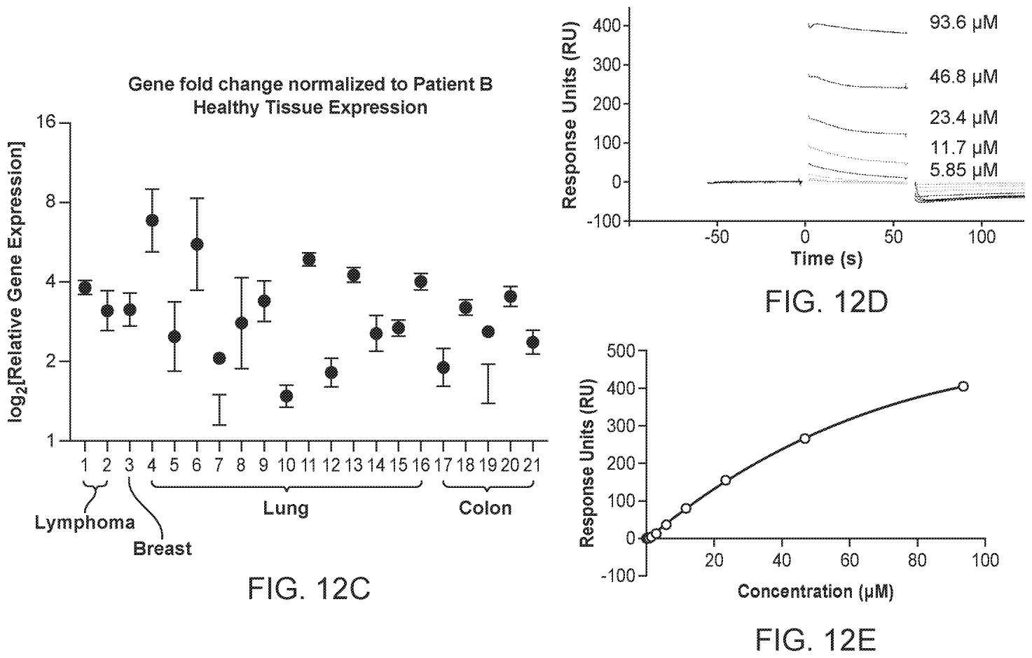

The deep learning method 2017DL was generated to consider peptides as whole entities rather than taking each individual position of the peptide as independent of every other, as the previous algorithms do (FIG. 12A). Sequencing data including peptide sequences and round counts were pre-processed in R to remove any peptide sequences that had fewer than 3 counts across all rounds. The data was then normalized by multiplying each round count by the average number of counts across the rounds and then divided by the number of counts in a given round. An adapted fitness score was used to score each peptide in the library derived from a fitness function represented by an exponential curve fit to each peptide through the normalized round counts (FIG. 12B).

Next a model was generated using the fitness scores for each peptide and the peptides represented as a 20 ×L matrix, where L is the length of the peptide sequence (FIG. 12C). The 20 rows of the matrix relate to the 20 possible amino acids. Amino acids are represented as a one-hot vector, in which a vector contains a single 1 with the remaining being 0s. The matrix representing the peptide was flattened to a feature vector of length 20×L for use in training the neural network. The one-hot matrix was used as input and the fitness scores used as output. A network architecture described previously utilizing a two-hidden layer network using 10 nodes and 5 nodes respectively was implemented using the data from the library peptides (FIG. 12D). The training was done in Lua with the Torch package. This model was used to score given peptides from the Uniprot database (downloaded Dec. 18, 2015) and patient-specific exomes using peptides isolated from an L-length sliding window converted to one-hot matrices for neural network input. P-values and Bonferroni-corrected p-values were calculated for each peptide, representing the probability of randomly selecting, from the whole proteome, a peptide with fitness score as high as or higher than the scored peptide.

Measuring T cell activation in co-culture assays. The four TCRs identified from the colorectal cancer patients that selected peptides from the library were cloned into a MSCV-based vector μMIG II in α-P2A-β configuration using the wildtype signal peptides of the TCR variable genes and full length, unmodified constant regions. The P2A skip sequence allows for 1:1 stoichiometric expression of the TCRs. A MSCV-based vector μMIG II was also used to generate human CD3 in the format of δ-F2A-γ-T2A-ε-P2A-ζ. A packaging vector pCL10A was used to incorporate env, gag, and pol to allow for human mammalian tropism and viral generation. The vectors introduced puromycin and zeocin selectivity into infected cells. Retrovirus was generated for each TCR and human CD3 in human embryonic kidney 293T cells using 5 μg TCR or human CD3 DNA and 3.3 μg pCL10A DNA. The viruses were generated using X-tremeGENE 9 DNA transfection reagent (Sigma-Aldrich) in serum-free DMEM. In cell culture, 2% FBS DMEM was used to recover the cells and media was changed at 12 hours. Virus was harvested at 36, 40, 44, and 48 hours each in 2.5 mL amounts to be pooled, filtered with 0.45 μM syringe filters (Fischer Scientific), and frozen at −80° C. or used immediately to infect TCR⁻CD8⁺ SKW-3 cells. The 2 mL virus of TCR and 2 mL virus of human CD3 was used to co-infect 2×10⁶ SKW-3 cells with 5 ug/mL polybrene (Millipore) by spinning for 2 hrs at 2500 rpm at 32° C. The virus was removed and replaced with media and cells cultured. The transduced SKW-3 cells were cultured after 2-3 days in 20 ug zeocin and 1 ug puromycin indefinitely to select for TCR and human CD3 co-expression. Cells were then co-stained for TCR (IP26, BioLegend) and human CD3 (UCHT1, BioLegend) and sorted on the SH800 cell sorter (Sony Biotechnology Inc.).

The transduced SKW-3 cells were co-cultured with TAP-deficient T2 cells in a 2:1 ratio with various peptide dilutions. The top 5 synthetic peptides isolated from the yeast-display selections were tested along with predictions determined from the 3 prediction algorithms. Peptides were synthesized to >70% purity (Genscript) (Elim Biopharm) and resuspended in dimethylsulfoxide to 20 mM and stored at −20° C. CD69 (FN50, BioLegend) was measured at 18 hours to detect early T cell activation by flow cytometry using the Accuri C6 (BD Biosciences). SKW-3 T cells were detected by UCHT1 staining and checked for TCR and CD3 expression. T2 cells were checked for HLA-A*02 expression by antibody (BB7.2, BioLegend). Data was analyzed using FlowJo version 10 (FlowJo, LLC) and samples were gated on SKW-3 cells by forward and side scatter and UCHT1+cells followed by analysis for CD69 expression. Experiments were done in biological triplicate and technical triplicate. P-values were calculated by ordinary one-way ANOVA in Prism and experiments plotted with either standard deviation or standard error of the mean as indicated.

CDK4-specific TCRs clone 10 (NKI1) and 17 (NKI2) were derived from TILs of a melanoma patient that were screened with HLA multimers loaded with predicted neoantigens, essentially as described. The variable parts of both TCRs were cloned into a retroviral vector encoding the murine TCR α and β constant domains. FLYRD18 packaging cells were plated in 10 cm dishes at 1.2×10⁶ cells/well. After one day, cells were transfected with 10 μg retroviral vector DNA encoding the CDK4 TCRs using 25 μl X-tremeGENE HP DNA (Sigma-Aldrich). After 48 hrs, retroviral supernatant was isolated and transferred to retronectin-coated 24-well plates and centrifuged for 90 minutes at 430g. PBMCs were activated and selected with anti-CD3/CD28 beads (ThermoFisher) at a bead-to-cell ratio of 3:1. Forty-eight hours after stimulation, T cells were plated at 0.5×10⁶ cells/mL on virus-coated plates. Surface expression of the introduced CDK4 TCRs on transduced T cells was measured using APC labeled CDK4 R>L HLA-A*02:01 tetramers in combination with anti-murine Vβ TCR-PE labeled antibody (BD Biosciences). Cells were analyzed using a FACSCalibur (Becton Dickinson). JY cells were pulsed with the CDK4 peptide or the predicted peptides at the indicated concentrations for 1 hr at 37° C. and then washed two times. Next, 0.2×10⁶ TCR-transduced T cells were incubated with 0.2×10⁶ peptide-pulsed JY cells in the presence of 1 μL/mL Golgiplug (BD Biosciences). T cells not exposed to JY cells, exposed to unloaded JY cells, and exposed to JY cells loaded with an irrelevant peptide (MART-1) were used as controls. After a 5-hour incubation at 37° C., 5% CO₂, cells were washed and stained with PerCP-cy5.5 anti-CD8, FITC anti-CD3, PE anti-murine Vβ TCR and APC anti-IFN₁ labeled antibodies.

Expression of refolded HLA-A*02:01 with exogenous peptide. The pet26b vector was used to express HLA-A*02:01 (1-275) and β2M (1-100) separately in Rosetta BL21 DE3 E. coli cells. Inclusion bodies containing the separate proteins were dissolved in 8 M urea, 40 mM Tris-HCl pH 8.0, 10 mM EDTA, and 10 mM DTT. For in vitro refolding, the HLA-A*02 heavy chain, β2M, and MMDFFNAQM (SEQ ID NO: 279) peptide were mixed in a 1:2:10 molar ratio and diluted into a refolding buffer containing 0.4 M L-arginine-HCl, 100 mM Tris-HCl pH 8.0, 4 mM EDTA, 0.5 mM oxidized glutathione, and 4 mM reduced glutathione. After 72 hours at 4° C., the protein was dialyzed in 10 L of 10 mM Tris-HCl and purified via weak ion exchange using a DEAE cellulose column. The protein elution was purified using size exclusion chromatography on a Superdex 200 column and ion-exchange chromatography on a 5/50 Mono Q column (GE Healthcare). Protein was biotinylated overnight with birA ligase, 100 uM biotin, 40 mM Bicine pH 8.3, 10 mM ATP, and 10 mM Magnesium Acetate at 4° C. after buffer-exchange to 1× HBS pH 7.2 in a 30 kDa filter (Millipore) before being run on a size exclusion Superdex 200 column.

Surface plasmon resonance to measure T CR 2A and 3B binding affinity to MMDFFNAQM-HLA-A*02:01. The interaction of TCR 2A and 3B with MMDFFNAQM-HLA-A*02 (SEQ ID NO: 281) was measured by surface plasmon resonance using a BIAcore T100 (GE Healthcare) biosensor at 25° C. Biotinylated MMDFFNAQM-HLA-A2 (SEQ ID NO: 282) was immobilized on a streptavidin-coated BIAcore SA chip at approximately 1000 resonance units (RU). A different flow cell was immobilized with non-relevant peptide-HLA-A2 to serve as blank control. Different concentrations of either 2A or 3B TCR were flowed sequentially over blank and MMDFFNAQM-HLA-A2 (SEQ ID NO: 282). Injections of TCR were stopped after 60 s to allow sufficient time for SPR signals to reach plateau. The dissociation constant (K_{D}) was obtained by fitting equilibrium data with a 1:1 binding model using BIAcore evaluation software.

Quantitative PCR to determine relative RNA expression of U2AF2. RNA extracted previously as mentioned above from the tumor and healthy patient tissue were used to determine the relative quantities of U2AF2 RNA expression. In addition, RNA was extracted from the following cell lines: Lymphoma: K562, Daudi; Breast: MDA MB 231; Lung: A549, EKVX, HCC78, H358, H441, H1373, H1437, H1650, H1792, H2009, H2126, H3122, LC-2/ad. cDNA was generated using the High-Capacity RNA-to-cDNA kit (Thermofisher) in triplicates. cDNA samples were pooled for quantity and quantitative real-time PCR carried out using TaqMan probes (ThermoFisher), TaqMan Universal Master Mix II, no UNG (ThermoFisher), and QuantStudio 3 Real-Time PCR System (ThermoFisher) in technical quadruplicate. The U2AF2 probe (ThermoFisher, Hs00200737_m1) amplified a 75 bp region spanning exons of U2AF2. The 18S RNA probe (ThermoFisher, Hs99999901_s1) was used as a housekeeping gene, amplifying a 187 bp region. The cycle threshold values of U2AF2 to 18S RNA were calculated for each sample and compared to either Patient A healthy tissue or Patient B healthy tissue cycle threshold values to determine relative expression levels. The standard deviation is plotted.

Quantification and statistical analysis. T-cell stimulation assays using SKW-3 cells. Data is analyzed using Flowjo to gate SKW-3 cells and CD3⁺ group to identify T cells. T cells are then gated on CD69 expression using the negative control (no peptide). The median MFI expression of CD69 in the CD3⁺ group and the percentage of cells expressing CD69 have been analyzed. One-way ordinary ANOVA was determined for both analyses using Prism in comparison to the negative control (no peptide). The 100 μM peptide stimulation is completed in biological and technical triplicate. Only one of the biological triplicates is shown. The peptide titration experiments were done in biological triplicate. All biological triplicates were analyzed collectively. Legends for p-value designations are listed for each figure. Either SEM (n=3; technical triplicate) or SD (n=3, biological replicate) are used and is listed in the corresponding figure legends.

2014PWM scoring. Scoring is done as presented in (Birnbaum et al., 2014). A frequency matrix is generated from the round 3 selection data using the sequencing read counts as a multiplier for peptide sequence. Each position of the peptide is multiplied by the read counts to get a count of the number of times a given amino acid is present. This is done for each unique peptide in round 3 and the amino acid counts per position is divided by the number of total reads. The frequency matrix is then used to score every Nmer peptide of the human proteome, in which N is the length of the selected peptides from the library. Scoring is done by multiplying the frequencies of the given amino acid across the peptide.

2017PWM and 2017DL peptide scoring. Algorithms were generated in this paper. For both the 2017PWM, a frequency matrix is generated as in 2014PWM, except an additional frequency matrix is generated for data across all rounds of selection, instead of just round 3. A ratio per position per amino acid is taken for round 3 frequency matrix to all round frequency matrix. A pseudocount frequency of 0.05 is implemented for zero values, and the log10 is taken of the ratio. This score is interpreted as the enrichment ratio of a particular amino acid at a position. This score is used to determine the overall enrichment of a given peptide from the exome or human proteome by multiplying scores for each position. The 2017DL algorithm is implemented as described in the methods.

To determine the statistical significance of a peptide, the human proteome and exome peptide set is scored. To calculate the p-values for the exome peptide set, the percentile score is calculated in context of the human proteome scores. The uncorrected p-value is 1-percentile. The Bonferroni-corrected p-value is the uncorrected p-value multiplied by the number of peptides in the mutant set.

Quantitative PCR analysis. Quantitative PCR was carried out in technical quadruplicate samples. The relative expression levels of U2AF2 RNA to 18S RNA (delta cycle threshold) was calculated by subtracting cycle threshold values. The fold-change over healthy (delta delta cycle threshold) was determined by subtracting the relative cycle threshold values (delta cycle threshold) of the reference to the sample. The standard deviation of a delta cycle threshold was calculated using

s=(s₁²+s₂²)^{1/2}

where s=standard deviation, s₁=standard deviation of target sample and s₂=standard deviation of reference sample. The delta delta cycle threshold standard deviation takes the standard deviation of the delta cycle threshold test sample.

Data and software availability. Exome sequencing. Data is available in the short read archive under BioSample accessions SAMN07350021, SAMN07350022, SAMN07350023, SAMN07350024, SAMN07350025, SAMN07350026, SAMN07350027, SAMN07350028, SAMN07350029, SAMN07350030, SAMN07350031, and SAMN07350032.

Deep-sequencing. Data is available in the short read archive under BioSample accessions SAMN07977164, SAMN07977165, SAMN07977166, SAMN07977167, SAMN07977168, and SAMN07977169.

TABLE 1
DMF5 selection data and human target prediction.

| | Cluster 1 | | Top 10 Cluster 2 |
|---|---|---|---|
| Cluster 1 Peptides | Predictions | Cluster 2 Peptides | Predictions |
| SMLGIGIVPV (SEQ | EAAGIGILTV | MMWDRGMGLL (SEQ | MLWDVQSGQM |
| ID NO: 283) | (SEQ ID NO: | ID NO: 322) | (SEQ ID NO: |
| | 313) | | 355) |
| SMAGIGIVDV (SEQ | TLGGIGLVTV | IMEDVGWLNV (SEQ | LLLQVGLSLL |
| ID NO: 284) | (SEQ ID NO: | ID NO: 323) | (SEQ ID NO: |
| | 314) | | 356) |
| NMGGLGIMPV (SEQ | ILLGIGIYAL | MMWDRGLGMM (SEQ | SLEDVVMLNV |
| ID NO: 285) | (SEQ ID NO: | ID NO: 324) | (SEQ ID NO: |
| | 315) | | 357) |
| NLSNLGILPV (SEQ | ILSGIGVSQV | ILEDRGFNQV (SEQ | MLEDRDLFVM |
| ID NO: 286) | (SEQ ID NO: | ID NO: 325) | (SEQ ID NO: |
| | 316) | | 358) |
| SMLGIGIYPV (SEQ | IMGNLGLIAV | LMFDRGMSLL (SEQ | MLEDMSLGIM |
| ID NO: 287) | (SEQ ID NO: | ID NO: 326) | (SEQ ID NO: |
| | 317) | | 359) |
| TMAGIGVHVV (SEQ | MAGNLGIITL | LMLDFDGSLL (SEQ | SLENRGLSML |
| ID NO: 288) | (SEQ ID NO: | ID NO: 327) | (SEQ ID NO: |
| | 318) | | 360) |
| SMAGIGTLVV (SEQ | IMGNLGLIVL | IMEDRGSLNM (SEQ | ILDDGGFLLM |
| ID NO: 289) | (SEQ ID NO: | ID NO: 328) | (SEQ ID NO: |
| | 319) | | 361) |
| SMSGLGILPM (SEQ | ILAGLGTSLL | LMNDMGFHIV (SEQ | LLWNFGLLIV |
| ID NO: 290) | (SEQ ID NO: | ID NO: 329) | (SEQ ID NO: |
| | 320) | | 362) |
| SMAGIGIVPV (SEQ | ELGGLKISTL | IMEDRGSGEM (SEQ | LLFDISFLML |
| ID NO: 291) | (SEQ ID NO: | ID NO: 330) | (SEQ ID NO: |
| | 321) | | 363) |
| SMLGIGIVDV (SEQ | | LMWDVGLSIM (SEQ | IMGDRNRNLL |
| ID NO: 292) | | ID NO: 331) | (SEQ ID NO: |
| | | | 364) |
| NMAGIGMGTV (SEQ | | SMWDRGTFIM (SEQ | |
| ID NO: 293) | | ID NO: 332) | |
| SMLGIGILPV (SEQ | | LMLDRGSPNM (SEQ | |
| ID NO: 294) | | ID NO: 333) | |
| SLSGIGISAV (SEQ | | IMFDRGIGIM (SEQ | |
| ID NO: 295) | | ID NO: 334) | |
| DLAGLGLYPV (SEQ | | ILFDRGMNLM (SEQ | |
| ID NO: 296) | | ID NO: 335) | |
| NMAGIGIIQV (SEQ | | MLLDRGLSLM (SEQ | |
| ID NO: 297) | | ID NO: 336) | |
| NMGGLGILPV (SEQ | | IMEDRGSLIL (SEQ | |
| ID NO: 298) | | ID NO: 337) | |
| SMAGIGIYPV (SEQ | | LMRDYQLLQV (SEQ | |
| ID NO: 299) | | ID NO: 338) | |
| NLSNLGIVPV (SEQ | | LMFDRGMSVL (SEQ | |
| ID NO: 300) | | ID NO: 339) | |
| IMLGIGIDTL (SEQ | | LMEDIGRELV (SEQ | |
| ID NO: 301) | | ID NO: 340) | |
| NLSNLGIMPV (SEQ | | ILEDRGMGLL (SEQ | |
| ID NO: 302) | | ID NO: 341) | |
| SMLGIGIVLV (SEQ | | MMDQFNGLMM (SEQ | |
| ID NO: 303) | | ID NO: 342) | |
| SMAGIGVHVV (SEQ | | IMWDRDYGVM (SEQ | |
| ID NO: 304) | | ID NO: 343) | |
| NMAGIGILTV (SEQ | | MMWDRGFNQV (SEQ | |
| ID NO: 305) | | ID NO: 344) | |
| MMAGIGIVDV (SEQ | | IMSMSVSNYL (SEQ | |
| ID NO: 306) | | ID NO: 345) | |
| NMGGLGIVPV (SEQ | | AMGDGSYLLM (SEQ | |
| ID NO: 307) | | ID NO: 346) | |
| SMLGIKIVPV (SEQ | | SMWDRGMGLL (SEQ | |
| ID NO: 308) | | ID NO: 347) | |
| ELSGLGIQTV (SEQ | | MMENRGSGAL (SEQ | |
| ID NO: 309) | | ID NO: 348) | |
| SMLGIGILPM (SEQ | | LMWDSGLELM (SEQ | |
| ID NO: 310) | | ID NO: 349) | |
| SMAGIGILPV (SEQ | | SMWDRGLGMM (SEQ | |
| ID NO: 311) | | ID NO: 350) | |
| SMLGIGIVPV (SEQ | | LMWDVGWLNV (SEQ | |
| ID NO: 312) | | ID NO: 351) | |
| | | MMWDRGTFIM (SEQ | |
| | | ID NO: 352) | |
| | | MMWDRGIVPV (SEQ | |
| | | ID NO: 353) | |
| | | ILFDRGMNLM (SEQ | |
| | | ID NO: 354) | |

The sequences identified from the round 3 deep-sequencing of the DMF5 10mer library selections after clustering by reverse hamming distance Using these clusters, predictions were made on the Uniprot database using 2014 PPM. The 9 predictions for the ‘GIG’ cluster and top 10 predictions for the ‘DRG’ clusler are listed.

TABLE 2

| NKI2 9mers | NKI2 10mers | | NKI2 11mers |
|---|---|---|---|
| VMISHENFM (SEQ | VMNGDSGTFL (SEQ | TLMSRSDLFL | ILSNRGHEVW (SEQ ID |
| ID NO: 365) | ID NO: 393) | (SEQ ID NO: | NO: 456) |
| | | 435) | |
| TMQSHEVML (SEQ | YMAVRSENFM (SEQ | ILNSRDEAMM | ILSNRGHENFMI (SEQ |
| ID NO: 366) | ID NO: 394) | (SEQ ID NO: | ID NO: 457) |
| | | 436) | |
| TMQSHENFM (SEQ | RMPNKQENFV (SEQ | ALNSRDEAMM | ILSNRGHDVFMI (SEQ |
| ID NO: 367) | ID NO: 395) | (SEQ ID NO: | ID NO: 458) |
| | | 437) | |
| VMQSHEVML (SEQ | IMDSKSEHFM (SEQ | ALDSRLEFFV | ILSNRGHEIFL (SEQ ID |
| ID NO: 368) | ID NO: 396) | (SEQ ID NO: | NO: 459) |
| | | 438) | |
| VMISHEIFL (SEQ | IMDSREEVFV (SEQ | VMDSRLEFFV | ILSNRGHEYFL (SEQ ID |
| ID NO: 369) | ID NO: 397) | (SEQ ID NO: | NO: 460) |
| | | 439) | |
| IMTSHEVML (SEQ | IMDSRSEHFM (SEQ | ALDSRSELFL | |
| ID NO: 370) | ID NO: 398) | (SEQ ID NO: | |
| | | 440) | |
| IMTSHEVMM (SEQ | GMDSRAEVFM (SEQ | AMYSNSDFMV | |
| ID NO: 371) | ID NO: 399) | (SEQ ID NO: | |
| | | 441) | |
| VMESHDVFM (SEQ | ALDSRSEYFL (SEQ | VMDSRLEHFM | |
| ID NO: 372) | ID NO: 400) | (SEQ ID NO: | |
| | | 442) | |
| IMNSHEVMM (SEQ | KMANRDENFV (SEQ | SMNSRSEHFM | |
| ID NO: 373) | ID NO: 401) | (SEQ ID NO: | |
| | | 443) | |
| SMNSHEVMM (SEQ | RLDGQDTKFM (SEQ | SMNSKSENFL | |
| ID NO: 374) | ID NO: 402) | (SEQ ID NO: | |
| | | 444) | |
| KMNSHEVMM (SEQ | LMDSRSEHFM (SEQ | VLDSSSSSFL | |
| ID NO: 375) | ID NO: 403) | (SEQ ID NO: | |
| | | 445) | |
| AMQGHEYFL (SEQ | IMNSRSELFL (SEQ | ALDSRSENFL | |
| ID NO: 376) | ID NO: 404) | (SEQ ID NO: | |
| | | 446) | |
| AMQGHEIFL (SEQ | MMNVRSELFV (SEQ | ALDSKSENFL | |
| ID NO: 377) | ID NO: 405) | (SEQ ID NO: | |
| | | 447) | |
| VLQSHEVSM (SEQ | TMNVRSELFV (SEQ | ALDSRSEIFL | |
| ID NO: 378) | ID NO: 406) | (SEQ ID NO: | |
| | | 448) | |
| AMQSHEVTL (SEQ | KMNSRSELFL (SEQ | SMNSRADMFV | |
| ID NO: 379) | ID NO: 407) | (SEQ ID NO: | |
| | | 449) | |
| LMSGDYQFV (SEQ | TMNVRSEHFM (SEQ | SMYSRQEMMV | |
| ID NO: 380) | ID NO: 408) | (SEQ ID NO: | |
| | | 450) | |
| TMHNHEVMM (SEQ | SMNSRSELFL (SEQ | RMWSRSEDMV | |
| ID NO: 381) | ID NO: 409) | (SEQ ID NO: | |
| | | 451) | |
| VMHNHEVMM (SEQ | KMNSRSEHFM (SEQ | VLRARSDVFV | |
| ID NO: 382) | ID NO: 410) | (SEQ ID NO: | |
| | | 452) | |
| TMTGHEVFM (SEQ | TMQSHDASFL (SEQ | ALDSREEVFV | |
| ID NO: 383) | ID NO: 411) | (SEQ ID NO: | |
| | | 453) | |
| TMTGHEVFV (SEQ | VMQGHDASFL (SEQ | SMNSREEIFL | |
| ID NO: 384) | ID NO: 412) | (SEQ ID NO: | |
| | | 454) | |
| VMQGHESFL (SEQ | KMNSHSGTFL (SEQ | SMSGFSESFV | |
| ID NO: 385) | ID NO: 413) | (SEQ ID NO: | |
| | | 455) | |
| VMISHEVML (SEQ | KMNGKSEDFM (SEQ | | |
| ID NO: 386) | ID NO: 414) | | |
| TMTGHEVML (SEQ | DMDNRLDRDM (SEQ | | |
| ID NO: 387) | ID NO: 415) | | |
| SMVGMEHSM (SEQ | IMDSKSEIFL (SEQ | | |
| ID NO: 388) | ID NO: 416) | | |
| AMQGHEHFM (SEQ | SMNSHSGTFL (SEQ | | |
| ID NO: 389) | ID NO: 417) | | |
| VMEGDYWFL (SEQ | SMNSREEHFM (SEQ | | |
| ID NO: 390) | ID NO: 418) | | |
| SMQSHEWML (SEQ | IMNSHSGTFL (SEQ | | |
| ID NO: 391) | ID NO: 419) | | |
| YMQTHESFM (SEQ | IMDSKSENFL (SEQ | | |
| ID NO: 392) | ID NO: 420) | | |
| | AMDSKSENFL (SEQ | | |
| | ID NO: 421) | | |
| | IMDSRADMFV (SEQ | | |
| | ID NO: 422) | | |
| | SMNSREEVFV (SEQ | | |
| | ID NO: 423) | | |
| | KMNSREEVFV (SEQ | | |
| | ID NO: 424) | | |
| | ALDSRSEHFM (SEQ | | |
| | ID NO: 425) | | |
| | AMDSRSEHFM (SEQ | | |
| | ID NO: 426) | | |
| | AMDSRADMFV (SEQ | | |
| | ID NO: 427) | | |
| | LMDSRSQIFV (SEQ | | |
| | ID NO: 428) | | |
| | GMTSRSDYMV (SEQ | | |
| | ID NO: 429) | | |
| | VMNSRSEHFM (SEQ | | |
| | ID NO: 430) | | |
| | VMNSRSDWFL (SEQ | | |
| | ID NO: 431) | | |
| | YMNSHDPYTV (SEQ | | |
| | ID NO: 432) | | |
| | RMDSRSQDFV (SEQ | | |
| | ID NO: 433) | | |
| | RMEAHSSHFV (SEQ | | |
| | ID NO: 434) | | |

Table 2. NKI2 selection data by peptide length.
The sequences identified from the round 3 deep-sequencing of the NKI2 library selections listed by peptide length. Related to FIG. 3.

TABLE 3
Patient HLA typing results.

| HLA | Patient A | Patient B |
|---|---|---|
| A | 2:01 | 2:01 |
| | 2:01 | 2:06 |
| B | 7:02 | 15:01 |
| | 15:01 | 35:01:00 |
| C | ND | ND |
| | ND | ND |
| DRB1 | 1:01 | 4:07 |
| | 4:04 | 4:07 |
| DRB345 | 4*01:01 | 4*01:01 |
| | ND | 4*01:01 |
| DQA | 1:01 | 3:01 |
| | 3:01 | 3:01 |
| DQB | 3:02 | 3:02 |
| | 5:01 | 3:02 |

TABLE 4
Patient A

| Tumor | Healthy | Vβ | CDR3β | Vα | CDR3α |
|---|---|---|---|---|---|
| 23 | 12 | TRBV7-2 | CASSLGLEQFF (SEQ ID | TRAV8-3 | CAGGGGADGLTF |
| | | | NO: 461) | | (SEQ ID NO: 470) |
| 6 | 0 | TRBV7-3 | CASSLGGGHTEAFF | TRAV19 | CALSEAEAAGNKLTF |
| | | | (SEQ ID NO: 462) | | (SEQ ID NO: 471) |
| 5 | 0 | TRBV7-9 | CASSLVNGLGYTF (SEQ | TRAV19 | CALSEAGMDSNYQLI |
| | | | ID NO: 463) | | W (SEQ ID NO: 472) |
| 4 | 0 | TRBV15 | CATSRDRGQDEKLFF | TRAV14/DV4 | CAMREGRYSGAGSY |
| | | | (SEQ ID NO: 464) | | QLTF (SEQ ID NO: |
| | | | | | 473) |
| 4 | 0 | TRBV9 | CASSADTGVNQPQHF | TRAV10 | CVVTETNAGKSTF |
| | | | (SEQ ID NO: 465) | | (SEQ ID NO: 474) |
| 4 | 0 | TRBV10-1 | CASSRDTVNTEAFF | TRAV19 | CALSEARGGATNKLIF |
| | | | (SEQ ID NO: 466) | | (SEQ ID NO: 475) |
| 1 | 0 | TRBV20-1 | CSARDYQGSQPQHF | TRAV12-2 | CAVNSGNTGKLIF |
| | | | (SEQ ID NO: 467) | | (SEQ ID NO: 476) |
| 1 | 0 | TRBV20-1 | CSARDYQGSQPQHF | TRAV20 | CAVPFLYNQGGKLIF |
| | | | (SEQ ID NO: 468) | | (SEQ ID NO: 477) |
| 1 | 0 | TRBV9 | CASSADTGVNQPQHF | TRAV12-2 | CAVNDFNKFYF (SEQ |
| | | | (SEQ ID NO: 469) | | ID NO: 478) |

Patient B

| Tumor | Healthy | Vβ | CDR3β | Vα | CDR3α |
|---|---|---|---|---|---|
| 35 | 0 | TRBV11-2 | CASSQGVGQFKNTQYF | TRAV12-2 | CAVETSNTGKLIF |
| | | | (SEQ ID NO: 479) | | (SEQ ID NO: 490) |
| 23 | 0 | TRBV7-2 | CASSLSGRQGGSYEQYF | TRAV29/DV5 | CAASSTGNQFYF |
| | | | (SEQ ID NO: 480) | | (SEQ ID NO: 491) |
| 21 | 0 | TRBV9 | CASSSSGGLVDTQYF | TRAV19 | CALSAGASGAGSYQL |
| | | | (SEQ ID NO: 481) | | TF (SEQ ID NO: 492) |
| 20 | 0 | TRBV2 | CASMGRSYGYTF (SEQ | TRAV39 | CALMNYGGATNKLIF |
| | | | ID NO: 482) | | (SEQ ID NO: 493) |
| 16 | 0 | TRBV11-3 | CASSLETGTAIYEQYF | TRAV13-1 | CAADNNNARLMF |
| | | | (SEQ ID NO: 483) | | (SEQ ID NO: 494) |
| 12 | 0 | TRBV11-3 | CASSPSGLAGSNLGNEQ | TRAV19 | CALSSRGSTLGRLYF |
| | | | FF (SEQ ID NO: 484) | | (SEQ ID NO: 495) |
| 11 | 0 | TRBV5-1 | CASSRIDSTDTQYF (SEQ | TRAV4 | CLVGEVGTASKLTF |
| | | | ID NO: 485) | | (SEQ ID NO: 496) |
| 10 | 0 | TRBV19 | CASSIPRGSSQPQHF | TRAV12-2 | CAVDSGGYNKLIF |
| | | | (SEQ ID NO: 486) | | (SEQ ID NO: 497) |
| 8 | 0 | TRBV10-3 | CAIKGGDRGVNTEAFF | TRAV14/DV4 | CAMREPNNAGNMLT |
| | | | (SEQ ID NO: 487) | | F (SEQ ID NO: 498) |
| 4 | 3 | TRBV20-1 | CSARLASYNEQFF (SEQ | TRAV12-2 | CAVRRATDSWGKLQ |
| | | | ID NO: 488) | | F (SEQ ID NO: 499) |
| 1 | 1 | TRBV10-1 | CASSRDFVSNEQYF | TRAV19 | CALSEARGGATNKLIF |
| | | | (SEQ ID NO: 489) | | (SEQ ID NO: 500) |

TCRs screened on the HLA-A*02:01 library. TCR sequences were chosen based on clonality in the tumor, phenotypic profile, exclusivity to the tumor, and additionally by related TCR sequences. The number beneath tumor and healthy labels indicate the number of times a paired TCR sequence was seen from this tissue. Related to FIGS. 5 and 6.

| SEQ ID | |
|---|---|
| NO | Sequence |
| 1. | LMDMHNGQL |
| 2. | RLDAMNGQL |
| 3. | RMDYNNMQM |
| 4. | SMDTFQGQM |
| 5. | GMDYHNGHL |
| 6. | YLDFHNGQL |
| 7. | LMDYTNMQL |
| 8. | NLDWANVQL |
| 9. | MMDLHNGQL |
| 10. | KMDYHEGQL |
| 11. | TLDGFNGQM |
| 12. | VMSHFEGQL |
| 13. | AMDYLNAQL |
| 14. | QLDWNNMQM |
| 15. | RMGYHNGQL |
| 16. | RMDRFNGQL |
| 17. | AMSYDNMQL |
| 18. | VMTHNNMQL |
| 19. | NMSWQNMQL |
| 20. | RMDVNNMQL |
| 21. | NLDWNNVQM |
| 22. | ELDWFNSQL |
| 23. | CMDVFNGQL |
| 24. | GMSYSNMQL |
| 25. | SMTWMNGQL |
| 26. | SMDRFNGQM |
| 27. | VLDQHNGQL |
| 28. | HMDFNNVQM |
| 29. | SMSWMNGQL |
| 30. | MLDWNNVQL |
| 31. | EMDVHNGQM |
| 32. | KMHWFNGQL |
| 33. | SMDSLNGQL |
| 34. | VMTYQNGQL |
| 35. | VMDHLNGQL |
| 36. | WMSDFQGQL |
| 37. | RLDSFNGQL |
| 38. | SMDSWNGQM |
| 39. | TMDWHSGQL |
| 40. | KLDIWNGQL |
| 41. | TMDFYQGQL |
| 42. | KMDYFSGQL |
| 43. | YLDYRNMQL |
| 44. | EMDHLNMQL |
| 45. | HMDINNMQM |
| 46. | SLDWFNSQL |
| 47. | RMDWLQAQL |
| 48. | FLDFRNGQM |
| 49. | EMMWWNGQV |
| 50. | TMEWFNGHL |
| 51. | TMDTLNAQL |
| 52. | FMDSFNGQM |
| 53. | NMMWFQGQL |
| 54. | NMGFENMQL |
| 55. | NMDYINVQL |
| 56. | EMDWSNLQL |
| 57. | LMGIHNGQL |
| 58. | EMSWFSGQL |
| 59. | VMDLFQGQM |
| 60. | LLDVHNMQL |
| 61. | KMDYNNVQM |
| 62. | SMDYNNVQM |
| 63. | LMENFQGQL |
| 64. | RMSFHNGQL |
| 65. | SMMYMNGQL |
| 66. | RMEWQNAQL |
| 67. | VMSHQNMQL |
| 68. | MMDFFDGQM |
| 69. | IMSHQNMQL |
| 70. | HMEFMNMQL |
| 71. | NMDTYNGQM |
| 72. | NLDYTNGQL |
| 73. | SMTWENMQL |
| 74. | AMTFHNGQL |
| 75. | SMDFTNAQM |
| 76. | NMSTRDERM |
| 77. | SMTFENMQL |
| 78. | EMDWWNGHL |
| 79. | TMDDNNGQL |
| 80. | LMDENNMQL |
| 81. | EMTNWNGQL |
| 82. | YMDYHNGHM |
| 83. | KMTWNNMQM |
| 84. | YMTHLNGQL |
| 85. | EMTWTNAQM |
| 86. | KMNNFEGQL |
| 87. | MMDLYNGQL |
| 88. | VLDNNNMQL |
| 89. | KLAWFNGQL |
| 90. | NLDHNNGQM |
| 91. | LMDNSNMQL |
| 92. | NMDYNNVQL |
| 93. | RMDYNNVQM |
| 94. | EMEIMNMQL |
| 95. | YMDRFQGQL |
| 96. | YMNVFEGQL |
| 97. | LMDTFNAQM |
| 98. | GMDYHNGQL |
| 99. | MLDLYNGQL |
| 100. | RLSWFQGQL |
| 101. | VLNGFDGQL |
| 102. | SMGWEQLQL |
| 103. | SMTWFTGQL |
| 104. | WMDISNMQL |
| 105. | TMQWQNAQL |
| 106. | SMTVFNGQL |
| 107. | NMDMHNMQL |
| 108. | RMSSFDGQL |
| 109. | YMSFDNVQL |
| 110. | LMSGFDGQL |
| 111. | YLDYLNMQL |
| 112. | SMDYNNIQM |
| 113. | GMDTHNGQL |
| 114. | LMDMHNGHL |
| 115. | SLNYWEGQL |
| 116. | ALNHFEGQL |
| 117. | AMDNMNGQL |
| 118. | RMGIFNGQL |
| 119. | NLDWSNAQL |
| 120. | RMDHMNGHL |
| 121. | MMSPFNGQL |
| 122. | TMNSWNGQL |
| 123. | SMNWQNGQL |
| 124. | IMETFNGQM |
| 125. | YLDNNNMQM |
| 126. | QMDLMKTYL |
| 127. | GLDWINGQL |
| 128. | RLTYLNGQL |
| 129. | AMDDWNGQM |
| 130. | NLDWQNMQM |
| 131. | TMDYNNAQM |
| 132. | TMDENNMQL |
| 133. | WMDDINGQL |
| 134. | MLDYMNAQM |
| 135. | AMDKHNGQM |
| 136. | KMDWRVVQM |
| 137. | RMDYTNMQL |
| 138. | RMDHSNMQM |
| 139. | TLEIHNGQL |
| 140. | LMDMHNMQM |
| 141. | SLTYFNGQM |
| 142. | YMDMHNGQL |
| 143. | NMDRHNGQM |
| 144. | NMDRNNMQL |
| 145. | TLDVHNMQL |
| 146. | RLSTFEGQL |
| 147. | QMDTMNGQL |
| 148. | KMDYHNGHL |
| 149. | IMDWSNVQM |
| 150. | KLDAFNGQM |
| 151. | CLSESLQWV |
| 152. | SMCYQNMQL |
| 153. | LMTCAGNDM |
| 154. | KLDVFNAQL |
| 155. | LMDYNNMQM |
| 156. | YLDFHNGHL |
| 157. | AMDMHNGQL |
| 158. | SMNYYDGQL |
| 159. | YMDWSNSQM |
| 160. | TLDHMNAQM |
| 161. | HMNYFDGQM |
| 162. | TLCYNNMQL |
| 163. | FMDDFSGQL |
| 164. | QLDWNNVQL |
| 165. | TLDFRNMQL |
| 166. | VLLRDASWM |
| 167. | TMEWFNGQM |
| 168. | FMDFNSGQL |
| 169. | SMDMHNGQL |
| 170. | RLQDISGVM |
| 171. | ELMAWNGQL |
| 172. | NLDWNNMQM |
| 173. | RMDYLNAQL |
| 174. | FMDFHNGQL |
| 175. | MMDLHNGHL |
| 176. | LMDTFQGQM |
| 177. | AMDFHNGQL |
| 178. | TMDFSNIQL |
| 179. | GMDDHNMQL |
| 180. | KMHYFNGQM |
| 181. | YMDYHNGQL |
| 182. | RMDYNNGHL |
| 183. | LMDYHEGQL |
| 184. | RMDRFNGQM |
| 185. | RMDVNNGQL |
| 186. | GMDTANMQL |
| 187. | MLDYMNGQL |
| 188. | KMTFHNAQL |
| 189. | FMDFNNVQM |
| 190. | SLDHFQGHL |
| 191. | TMDFYQGQL |
| 192. | KMDYFSGQL |
| 193. | SMDWFQGQM |
| 194. | LMDYWQGQL |
| 195. | NMMWFQGQL |
| 196. | KMHWFNGQL |
| 197. | TMDYWQGHL |
| 198. | RMDRFNGQL |
| 199. | SMDTFQGQM |
| 200. | VMSHFEGQL |
| 201. | LMDYTNMQL |
| 202. | KMDYHIGQM |
| 203. | VMDHFQAQL |
| 204. | NMGFENMQL |
| 205. | YLDHKTLRL |
| 206. | TMDYWQGQL |
| 207. | KMRMNRHKL |
| 208. | YMDRFQGQM |
| 209. | SMDFFNSQL |
| 210. | NMEEYCALV |
| 211. | SMDFYQGQL |
| 212. | SMDWFQGQL |
| 213. | NMMWFQGQM |
| 214. | AMYKLSGLM |
| 215. | HMEYRYANM |
| 216. | LMDYFSGQL |
| 217. | TMDWFQGQM |
| 218. | FMSVAKFVV |
| 219. | RLDYHNMQL |
| 220. | LMDFYQGQL |
| 221. | LMDYWQGHL |
| 222. | TMDFYQGQM |
| 223. | KMLSIDVVM |
| 224. | SMDYFSGQL |
| 225. | KMKNHHTKV |
| 226. | SMDYWQGQL |
| 227. | KLHRHKQHM |
| 228. | LMDWFQGQM |
| 229. | KMTSWWDML |
| 230. | DMDWFQGQM |
| 231. | MLYELTEHL |
| 232. | SMDWFNGQL |
| 233. | RLHRRDNLM |
| 234. | DMDYWQGQL |
| 235. | KMDYTNMQL |
| 236. | TMDYWQGQM |
| 237. | FMGVSYEMM |
| 238. | LMDYWQGQM |
| 239. | SMDTFQGQL |
| 240. | KMHGHKHYM |
| 241. | KMHWFQGQM |
| 242. | SLDYFNSQL |
| 243. | YMDRFQGQL |
| 244. | RMWSDRMDL |
| 245. | KMDYFNSQL |
| 246. | YMHSHSVLL |
| 247. | DMDYFSGQL |
| 248. | SMDWFQGHL |
| 249. | VMDLFQGQM |
| 250. | NMESWLSMM |
| 251. | RMDRFQGQM |
| 252. | SMEISNLNM |
| 253. | DMERALMNL |
| 254. | DMDTFQGQM |
| 255. | KMKKNHDHM |
| 256. | KMREMPVKM |
| 257. | MMDFFNAQM |

TCR 2A:
TCR comprised of TRAV19, TRAJ32, CDR3: (SEQ ID NO:
261)
CALSEARGGATNKLIF
and
TRBV10-1, TRBJ1-1, CDR3: (SEQ ID NO: 262)
CASSRDTVNTEAFF
alpha chain: (SEQ ID NO: 258)
QKVTQAQTEISVVEKEDVTLDCVYETRDTTYYLFWYKQPPSGELVFLIRR
NSFDEQNEISGRYSWNFQKSTSSFNFTITASQVVDSAVYFCALSEARGGA
TNKLIFGTGTLLAVQPNIQNPDPAVYQLRDSKSSDKSVCLFTDFDSQTNV
SQSKDSDVYITDKCVLDMRSMDFKSNSAVAWSNKSDFACANAFNNSIIPE
DTFFPSPESS
beta chain (SEQ ID NO: 259)
EITQSPRHKITETGRQVTLACHQTWNHNNMFWYRQDLGHGLRLIHYSYGV
QDTNKGEVSDGYSVSRSNTEDLPLTLESAASSQTSVYFCASSRDTVNTEA
FFGQGTRLTVVEDLKNVFPPEVAVFEPSEAEISHTQKATLVCLATGFYPD
HVELSWWVNGKEVHSGVCTDPQPLKEQPALNDSRYALSSRLRVSATFWQN
PRNHFRCQVQFYGLSENDEWTQDRAKPVTQIVSAEAWGRAD
TCR3B:
TCR comprised of TRAV19, TRAJ32, CDR3: (SEQ ID NO:
261)
CALSEARGGATNKLIF
and
TRBV10-1, TRBJ2-7, CDR3: (SEQ ID NO: 263)
CASSRDFVSNEQYF
alpha same as TCR 2A
beta chain (SEQ ID NO: 260)
EITQSPRHKITETGRQVTLACHQTWNHNNMFWYRQDLGHGLRLIHYSYGV
QDTNKGEVSDGYSVSRSNTEDLPLTLESAASSQTSVYFCASSRDFVSNEQ
YFGPGTRLTVTEDLKNVFPPEVAVFEPSEAEISHTQKATLVCLATGFYPD
HVELSWWVNGKEVHSGVCTDPQPLKEQPALNDSRYALSSRLRVSATFWQN
PRNHFRCQVQFYGLSENDEWTQDRAKPVTQIVSAEAWGRAD

What is claimed is:

1. A peptide comprising an amino acid sequence of any of SEQ ID NO:1-SEQ ID NO:257 or SEQ ID NO:262.

2. A peptide consisting of an amino acid sequence of any of SEQ ID NO:1-SEQ ID NO:257 or SEQ ID NO:262.

3. A polynucleotide encoding a peptide of claim 1 or claim 2.

4. A pharmaceutical composition comprising polynucleotide, a peptide or combination of peptides of any of claims 1-3; and a pharmaceutically acceptable excipient.

5. A pharmaceutical composition of claim 4, comprising a vaccine adjuvant.

6. A pharmaceutical composition of claim 4 or claim 5, wherein the peptide or combination of peptides is complexed with an MHC antigen.

7. An antigen presenting cell comprising a peptide or combination of peptides of claim 1 or claim 2.

8. A method of inducing an immune response to a cancer cell antigen, the method comprising:
administering an individual an effective dose of a pharmaceutical formulation of any of claims 4-6, or an antigen presenting cell of claim 7.

9. A T cell receptor or antibody comprising the CDR sequences of any of SEQ ID NO:258, 259 or 260.

10. The T cell receptor of claim 9, comprising the amino acid sequence of SEQ ID NO:258, paired with the sequence of SEQ ID NO:259 or SEQ ID NO:260.

11. An immune cell engineered to comprise a T cell receptor or antibody of claim 9 or claim 10.

12. A method of determining the responsiveness of an individual to an antigen, the method comprising:
analyzing a sample comprising T cells from the individual for T cell stimulation in response to a peptide according to any SEQ ID NO:1-257 or 262; wherein T cell stimulation in response to the peptide is indicative that the individual can be treated according to the method of claim 8.

13. A peptide antigen for a TCR, identified by the method comprising:
contacting a TCR of interest with a population of host cells, which express on the cell surface a multiplexed library of at least 10⁸ different polynucleotides encoding single chain polypeptides, the single chain polypeptides comprising:
binding domains of the MHC protein; and
a peptide ligand;
selecting for host cells expressing a single chain polypeptide that binds to the TCR of interest;
iterating the selecting step for at least three rounds;
performing DNA sequencing of the polynucleotides present in the final selected population to determine a dataset of possible amino acids for each position of the peptide ligand;
inputting the dataset to computer readable medium to generate a search algorithm;
searching a sequence database with the search algorithm to identify the set of peptides that bind to the T cell receptor.

14. The peptide antigen of claim 13, wherein the peptide ligand is from 8 to 20 amino acids in length.

15. The peptide antigen of claim 14, wherein the library contains peptide ligand randomized at multiple positions.

16. The peptide antigen of claim 15, wherein the library of peptide ligands has limited diversity at the MHC anchor positions.

17. The peptide antigen of any one of claims 13-16, wherein the MHC binding domains comprise the alpha 1 and alpha 2 domains of a Class I MHC protein and β2 microglobulin.

18. The peptide antigen of claim 5, wherein the Class I MHC is an allele of HLA-A2.

19. The peptide antigen of claim 14, wherein the HLA-A2 allele comprises the amino acid change {Y84A}.

20. A method of screening for peptide antigen of a TCR, the method comprising:
contacting a TCR of interest with a population of host cells, which express on the cell surface a multiplexed library of at least 10⁸ different polynucleotides encoding single chain polypeptides, the single chain polypeptides comprising:
binding domains of the MHC protein; and
a peptide ligand;
selecting for host cells expressing a single chain polypeptide that binds to the TCR of interest;
iterating the selecting step for at least three rounds;
performing DNA sequencing of the polynucleotides present in the final selected population to determine a dataset of possible amino acids for each position of the peptide ligand;
inputting the dataset to computer readable medium to generate a search algorithm;
searching a sequence database with the search algorithm to identify the set of peptides that bind to the T cell receptor.
